SEQUENCE LISTING (30 sequences)

SEQ ID NO: 1 - DNA, 717 bp, Artificial
  TRX1 light chain
atggagacagacacaatcctgctatgggtgctgctgctctgggttccaggctccactggtgacattgtgatgacccaatctccagattctttggctgtgtctctaggtgagagggccaccatcaactgcaaggccagccaaagtgttgattatgatggtgatagttatatgaactggtatcaacagaaaccaggacagccacccaaactcctcatctatgttgcatccaatctagagtctggggtcccagacaggtttagtggcagtgggtctgggacagacttcaccctcaccatcagttctctgcaggcggaggatgttgcagtctattactgtcagcaaagtcttcaggaccctccgacgttcggtggaggtaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - protein, 238 aa, Artificial
  TRX1 light chain
METDTILLWVLLLWVPGSTGDIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 218 aa, Artificial
  TRX1 light chain
DIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - DNA, 1404 bp, Artificial
  TRX1 heavy chain
atggaatggatctggatctttctcctcatcctgtcaggaactcgaggtgtccagtcccaggttcagctggtgcagtctggagctgaagtgaagaagcctggggcttcagtgaaggtgtcctgtaaggcttctggatacacattcactgcctatgttataagctgggtgaggcaggcacctggacagggccttgagtggatgggagagatttatcctggaagcggtagtagttattataatgagaagttcaagggcagggtcacaatgactagagacacatccaccagcacagtctacatggaactcagcagcctgaggtctgaggacactgcggtctattactgtgcaagatccggggacggcagtcggtttgtttactggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 5 - protein, 467 aa, Artificial
  TRX1 heavy chain
MEWIWIFLLILSGTRGVQSQVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 448 aa, Artificial
  TRX1 heavy chain
QVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 717 bp, Artificial
  TRX1 light chain
atggagacagacacaatcctgctatgggtgctgctgctctgggttccaggctccactggtgacattgtgatgacccaatctccagattctttggctgtgtctctaggtgagagggccaccatcaactgcaaggccagccaaagtgttgattatgatggtgatagttatatgaactggtatcaacagaaaccaggacagccacccaaactcctcatctatgttgcatccaatctagagtctggggtcccagacaggtttagtggcagtgggtctgggacagacttcaccctcaccatcagttctctgcaggcggaggatgttgcagtctattactgtcagcaaagtcttcaggaccctccgacgttcggtggaggtaccaaggtggaaatcaaacgaactgtggctgcactatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 8 - protein, 238 aa, Artificial
  TRX1 light chain
METDTILLWVLLLWVPGSTGDIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAALSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 218 aa, Artificial
  TRX1 light chain
DIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAALSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 1404 bp, Artificial
  TRX1 heavy chain
atggaatggatctggatctttctcctcatcctgtcaggaactcgaggtgtccagtcccaggttcagctggtgcagtctggagctgaagtgaagaagcctggggcttcagtgaaggtgtcctgtaaggcttctggatacacattcactgcctatgttataagctgggtgaggcaggcacctggacagggccttgagtggatgggagagatttatcctggaagcggtagtagttattataatgagaagttcaagggcagggtcacaatgactagagacacatccaccagcacagtctacatggaactcagcagcctgaggtctgaggacactgcggtctattactgtgcaagatccggggacggcagtcggtttgtttactggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 11 - protein, 467 aa, Artificial
  TRX1 heavy chain
MEWIWIFLLILSGTRGVQSQVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 448 aa, Artificial
  TRX1 heavy chain
QVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 717 bp, Artificial; identical to SEQ ID NO: 7
  TRX1 light chain

SEQ ID NO: 14 - protein, 238 aa, Artificial; identical to SEQ ID NO: 8
  TRX1 light chain

SEQ ID NO: 15 - protein, 218 aa, Artificial; identical to SEQ ID NO: 9
  TRX1 light chain

SEQ ID NO: 16 - DNA, 1404 bp, Artificial; identical to SEQ ID NO: 4
  TRX1 heavy chain

SEQ ID NO: 17 - protein, 467 aa, Artificial; identical to SEQ ID NO: 5
  TRX1 heavy chain

SEQ ID NO: 18 - protein, 448 aa, Artificial; identical to SEQ ID NO: 6
  TRX1 heavy chain

SEQ ID NO: 19 - DNA, 717 bp, Artificial; identical to SEQ ID NO: 1
  TRX1 light chain

SEQ ID NO: 20 - protein, 238 aa, Artificial; identical to SEQ ID NO: 2
  TRX1 light chain

SEQ ID NO: 21 - protein, 218 aa, Artificial; identical to SEQ ID NO: 3
  TRX1 light chain

SEQ ID NO: 22 - DNA, 1404 bp, Artificial; identical to SEQ ID NO: 10
  TRX1 heavy chain

SEQ ID NO: 23 - protein, 467 aa, Artificial; identical to SEQ ID NO: 11
  TRX1 heavy chain

SEQ ID NO: 24 - protein, 448 aa, Artificial; identical to SEQ ID NO: 12
  TRX1 heavy chain

SEQ ID NO: 25 - protein, 15 aa, Artificial
  TRX1 light chain CDR1
KASQSVDYDGDSYMN

SEQ ID NO: 26 - protein, 7 aa, Artificial
  TRX1 light chain CDR2
VASNLES

SEQ ID NO: 27 - protein, 9 aa, Artificial
  TRX1 light chain CDR3
QQSLQDPPT

SEQ ID NO: 28 - protein, 5 aa, Artificial
  TRX1 heavy chain CDR1
AYVIS

SEQ ID NO: 29 - protein, 17 aa, Artificial
  TRX1 heavy chain CDR2
EIYPGSGSSYYNEKFKG

SEQ ID NO: 30 - protein, 9 aa, Artificial
  TRX1 heavy chain CDR3
SGDGSRFVY

(19) United States
(12) Patent Application Publication
(10) Pub. No.: US 2008/0279848 A1
(43) Pub. Date: Nov. 13, 2008

(54) Methods of treating lupus using CD4 antibodies

(75) Inventor: Bryan Irving, San Francisco, CA (US)

Correspondence Address: QUINE INTELLECTUAL PROPERTY LAW GROUP, P.C., P O BOX 458, ALAMEDA, CA, 94501, US

(73) Assignee: GENENTECH, INC., South San Francisco, CA (US)

(21) Appl. No.: 12/077,934

(22) Filed: Mar. 20, 2008

Related U.S. Application Data
(63) Continuation-in-part of application No. 11/724,595, filed on Mar. 14, 2007.
(60) Provisional application No. 60/919,505, filed on Mar. 21, 2007.
(60) Provisional application No. 60/783,535, filed on Mar. 16, 2006.
(60) Provisional application No. 60/873,881, filed on Dec. 7, 2006.

(51) Int. Cl. A61K 39/395; A61P 37/00

(52) U.S. Cl. 424/133.1; 424/130.1

(57) ABSTRACT

Methods of treating lupus, including systemic lupus erythematosus, cutaneous lupus erythmetosus, and lupus nephritis, are provided. The methods involve administration of a combination of a non-depleting CD4 antibody and another compound used clinically or experimentally to treat lupus. Methods of treating lupus nephritis by administration of a non-depleting CD4 antibody that results in an improvement in renal function and/or a reduction in proteinuria or active urinary sediment are also provided. Methods of treating lupus or decreasing autoantibody titer by administration of a non-depleting CD4 antibody are also provided. Methods of treating multiple sclerosis by administration of a non-depleting CD4 antibody, optionally in combination with another compound used clinically or experimentally to treat MS, are described. Methods of treating transplant recipients and subjects with rheumatoid arthritis, asthma, psoriasis, Crohn's disease, ulcerative colitis, and Sjogren's syndrome are also provided.

### CROSS-REFERENCE TO RELATED APPLICATIONS

This application is a non-provisional utility patent application which claims priority to and benefit of provisional patent application U.S. Ser. No. 60/919,505, filed Mar. 21, 2007, entitled “METHODS OF TREATING LUPUS USING CD4 ANTIBODIES” by Bryan Irving, and which is a continuation-in-part of U.S. patent application Ser. No. 11/724,595, filed Mar. 14, 2007, entitled “METHODS OF TREATING LUPUS USING CD4 ANTIBODIES” by Bryan Irving, which claims priority to and benefit of provisional patent applications U.S. Ser. No. 60/783,535, filed Mar. 16, 2006, entitled “METHODS OF TREATING LUPUS USING CD4 ANTIBODIES” by Bryan Irving, and U.S. Ser. No. 60/873,881, filed Dec. 7, 2006, entitled “METHODS OF TREATING LUPUS USING CD4 ANTIBODIES” by Bryan Irving. Each of these applications is incorporated herein by reference in its entirety for all purposes.

### FIELD OF THE INVENTION

The invention relates to methods of treating lupus and other autoimmune disorders in mammalian subjects using non-depleting CD4 antibodies, alone or in combination with other compounds.

### BACKGROUND OF THE INVENTION

Autoimmune diseases, such as systemic lupus erythematosus (SLE), myasthenia gravis, multiple sclerosis, and idiopathic thrombocytopenic purpura, among others, remain clinically important diseases in humans. As the name implies, autoimmune diseases wreak their havoc through the body's own immune system. While the pathological mechanisms differ between individual types of autoimmune diseases, one general mechanism involves the binding of certain antibodies (referred to herein as self-reactive antibodies or autoantibodies) present in the sera of patients to self-nuclear or cellular antigens.

Lupus is an autoimmune disease involving antibodies that attack connective tissue. The disease is estimated to affect nearly 1 million Americans, primarily women between the ages of 20-40. The principal form of lupus is a systemic one (systemic lupus erythematosus; SLE). SLE is associated with the production of antinuclear antibodies, circulating immune complexes, and activation of the complement system. SLE has an incidence of about 1 in 700 women between the ages of 20 and 60. SLE can affect any organ system and can cause severe tissue damage. Numerous autoantibodies of differing specificity are present in SLE. SLE patients often produce autoantibodies having anti-DNA, anti-Ro, and anti-platelet specificity and that are capable of initiating clinical features of the disease, such as glomerulonephritis, arthritis, serositis, complete heart block in newborns, and hematologic abnormalities. These autoantibodies are also possibly related to central nervous system disturbances. Arbuckle et al. describes the development of autoantibodies before the clinical onset of SLE (Arbuckle et al. (2003) N. Engl. J. Med. 349(16):1526-1533). The presence of antibodies immunoreactive with double-stranded native DNA is frequently used as a diagnostic marker for SLE.

Untreated lupus can be fatal as it progresses from attack of skin and joints to internal organs, including lung, heart, and kidneys (with renal disease being the primary concern). Lupus mainly appears as a series of flare-ups, with intervening periods of little or no disease manifestation. Kidney damage, measured by the amount of proteinuria in the urine, is one of the most acute areas of damage associated with pathogenicity in SLE, and accounts for at least 50% of the mortality and morbidity of the disease.

Currently, there are no curative treatments for patients who have been diagnosed with SLE. From a practical standpoint, physicians generally employ a number of powerful immunosuppressive drugs such as high-dose corticosteroids, e.g., prednisone, or azathioprine or cyclophosphamide, which are given during periods of flare-ups, but which may also be given persistently for those who have experienced frequent flare-ups. Even with effective treatment, which reduces symptoms and prolongs life, many of these drugs have potentially harmful side effects to the patients being treated. In addition, these immunosuppressive drugs interfere with the person's ability to produce all antibodies, not just the self-reactive anti-DNA antibodies. Immunosuppressants also weaken the body's defense against other potential pathogens, thereby making the patient extremely susceptible to infection and other potentially fatal diseases, such as cancer. In some of these instances, the side effects of current treatment modalities, combined with continued low-level manifestation of the disease, can cause serious impairment and premature death.

Recent therapeutic regimens include cyclophosphamide, methotrexate, antimalarials, hormonal treatment (e.g., DHEA), and anti-hormonal therapy (e.g., the anti-prolactin agent bromocriptine). Methods for treatment of SLE involving antibodies are also described. For example, the method in Diamond et al. (U.S. Pat. No. 4,690,905) consists of generating monoclonal antibodies against anti-DNA antibodies (the monoclonal antibodies being referred to therein as anti-idiotypic antibodies) and then using these anti-idiotypic antibodies to remove the pathogenic anti-DNA antibodies from the patient's system. However, the removal of large quantities of blood for treatment can be a dangerous, complicated process. U.S. Pat. No. 6,726,909 discloses treating SLE wherein the antibody composition administered to the patient comprises purified anti-DNA anti-idiotypic antibodies and the administration requires an injection, or other equivalent mode of administration.

High-dose intravenous immune globulin (IVIG) infusions have also been used in treating certain autoimmune diseases. Up until the present time, treatment of SLE with IVIG has provided mixed results, including both resolution of lupus nephritis (Akashi et al., J. Rheumatology 17:375-379 (1990)), and in a few instances, exacerbation of proteinuria and kidney damage (Jordan et al., Clin. Immunol. Immunopathol. 53: S164-169 (1989)).

Persons afflicted with lupus such as those with SLE who show clinical evidence for lupus nephritis and those with lupus nephritis need a cost-efficient and safe treatment that will help ameliorate the tissue damage that leads ultimately to kidney failure and the need for chronic hemodialysis and/or renal transplantation caused by their condition. Similarly, persons afflicted with other autoimmune diseases, such as multiple sclerosis (MS), rheumatoid arthritis, myasthenia gravis, psoriasis, juvenile onset diabetes, Sjogren's disease, thyroid disease, and inflammatory bowel disease also need effective and safe treatments.

### SUMMARY OF THE INVENTION

One general class of embodiments provides methods of treating lupus in a mammalian subject, e.g., a human subject. In the methods, a therapeutically effective amount of a combination of a non-depleting CD4 antibody and at least a second compound selected from, e.g., the group consisting of cyclophosphamide, mycophenolate mofetil, CTLA4-Ig, and an α4-integrin antibody, etc. is administered to the subject. In certain embodiments, the subject is a human. In certain embodiments, the second compound is cyclophosphamide.

In one class of embodiments, the non-depleting CD4 antibody has a light chain amino acid sequence set forth in SEQ ID NO:3 and a heavy chain amino acid sequence set forth in SEQ ID NO:6, a light chain amino acid sequence set forth in SEQ ID NO:9 and a heavy chain amino acid sequence set forth in SEQ ID NO:12, a light chain amino acid sequence set forth in SEQ ID NO:15 and a heavy chain amino acid sequence set forth in SEQ ID NO:18, or a light chain amino acid sequence set forth in SEQ ID NO:21 and a heavy chain amino acid sequence set forth in SEQ ID NO:24.

In one class of embodiments, the non-depleting CD4 antibody comprises a CD4 binding fragment of an antibody that comprises a light chain amino acid sequence set forth in SEQ ID NO:3 and a heavy chain amino acid sequence set forth in SEQ ID NO:6, a light chain amino acid sequence set forth in SEQ ID NO:9 and a heavy chain amino acid sequence set forth in SEQ ID NO:12, a light chain amino acid sequence set forth in SEQ ID NO:15 and a heavy chain amino acid sequence set forth in SEQ ID NO:18, or a light chain amino acid sequence set forth in SEQ ID NO:21 and a heavy chain amino acid sequence set forth in SEQ ID NO:24.

In one class of embodiments, the non-depleting CD4 antibody comprises CDR1 (SEQ ID NO:25), CDR2 (SEQ ID NO:26), or preferably CDR3 (SEQ ID NO:27) of the light chain shown in FIG. 1A; for example, the antibody can include CDR1, CDR2, and CDR3 of the light chain shown in FIG. 1A (i.e., SEQ ID NO:25, SEQ ID NO:26, and SEQ ID NO:27). Similarly, in one class of embodiments, the antibody comprises CDR1 (SEQ ID NO:28), CDR2 (SEQ ID NO:29), or preferably CDR3 (SEQ ID NO:30) of the heavy chain shown in FIG. 1D; for example, the antibody can include CDR1, CDR2, and CDR3 of the heavy chain shown in FIG. 1D (i.e., SEQ ID NO:28, SEQ ID NO:29, and SEQ ID NO:30). In one embodiment, the antibody comprises CDR1, CDR2, and CDR3 of the light chain shown in FIG. 1A and CDR1, CDR2, and CDR3 of the heavy chain shown in FIG. 1D (i.e., SEQ ID NOs:25-30). Other exemplary antibodies include, but are not limited to, antibodies that bind the same epitope as an antibody shown in any one of FIGS. 1-4.

The non-depleting CD4 antibody can be a humanized antibody, e.g., where the subject to be treated is a human. The antibody can have an aglycosylated Fc portion. Optionally, the antibody does not bind to the Fc receptor. In certain embodiments, the antibody is an anti-CD4 variant antibody that can bind an FcRN receptor. The antibody optionally includes an amino acid substitution at one or more of amino acid positions 270, 322, 326, 327, 329, 313, 333, and/or 334 of the Fc region altering C1q binding and/or complement-dependent cytotoxicity of the antibody (e.g., with respect to a parental antibody not including such substitution). In certain embodiments, the antibody comprises a salvage receptor binding epitope or a serum albumin binding peptide. Optionally, the antibody comprises three or more antigen-binding sites.

The lupus for which the subject is treated is typically systemic lupus erythematosus (SLE), cutaneous lupus erythematosus (CLE), or lupus nephritis. The lupus to be treated can be early, mid, or late stage disease when treatment is initiated. In embodiments in which lupus nephritis is treated, the lupus nephritis can be any one of classes I-VI. For example, the lupus to be treated can be class II lupus nephritis, class III lupus nephritis, class IV lupus nephritis, or class V lupus nephritis. In one embodiment, after initiation of treatment with the combination, the subject displays a reduction in proteinuria and/or a reduction in active urinary sediment, as compared to the level(s) of proteinuria and/or active urinary sediment displayed by the subject prior to initiation of treatment. For example, proteinuria can be reduced by at least 25%, by at least 50%, by at least 75%, or by at least 90%, or the proteinuria can be reduced to less than 1 g per day or less than 500 mg per day, and/or active urinary sediment can be reduced by at least 25%, by at least 50%, by at least 75%, or by at least 90%, or only inactive urinary sediment may remain after initiation of treatment.

In one embodiment, prior to initiation of treatment with the combination, the subject displays proteinuria, which proteinuria is ameliorated by the treatment. For example, prior to initiation of treatment, the subject can display proteinuria greater than 500 mg per day, greater than 1000 mg per day, greater than 2000 mg per day, or greater than 3500 mg per day. After initiation of treatment, proteinuria can be reduced by at least 25%, by at least 50%, by at least 75%, or by at least 90%, or the proteinuria can be reduced to less than 1 g per day or less than 500 mg per day. As another example, prior to initiation of treatment, the subject can display a protein to creatinine ratio greater than 0.5, greater than 1, or greater than 2; after initiation of treatment, the subject's urine protein to creatinine ratio can be reduced by at least 25% or by at least 50%, or the ratio can be reduced to less than 1 or less than 0.5.

In one embodiment, after initiation of treatment with the combination, the subject displays a reduction in anti-double-stranded DNA antibody titer, e.g., by at least 25%, by at least 50%, or by at least 75%, as compared to the anti-double-stranded DNA antibody titer displayed by the subject prior to initiation of treatment. Optionally, the second compound in the combination is mycophenolate mofetil.

In one aspect, the methods include treating the subject with the non-depleting CD4 antibody and the second compound to reduce symptoms, and then continuing treatment of the subject with the non-depleting CD4 antibody or with the second compound (not in combination with each other) to maintain remission. For example, in one class of embodiments, after initiation of treatment with the combination, the lupus is ameliorated; treatment of the subject with the combination is then discontinued, and instead a therapeutically effective amount of the non-depleting CD4 antibody is administered to the subject. In another exemplary class of embodiments, after initiation of treatment with the combination, the lupus is ameliorated; treatment of the subject with the combination is then discontinued, and instead a therapeutically effective amount of the second compound or one or more other compounds is administered to the subject.

Another general class of embodiments also provides methods of treating lupus nephritis in a mammalian subject, e.g., a human. In the methods, a therapeutically effective amount of a non-depleting CD4 antibody is administered to the subject. After initiation of treatment with the non-depleting antibody, the subject displays an improvement in renal function, a reduction in proteinuria, and/or a reduction in active urinary sediment, as compared to the level(s) of proteinuria and/or active urinary sediment displayed by the subject prior to initiation of treatment. For example, proteinuria can be reduced by at least 25%, by at least 50%, by at least 75%, or by at least 90%, or the proteinuria can be reduced to less than 1 g per day or less than 500 mg per day; protein to creatinine ratio can be reduced by at least 25% or by at least 50%, or the ratio can be reduced to less than 1 or less than 0.5; and/or active urinary sediment can be reduced by at least 25%, by at least 50%, by at least 75%, or by at least 90%, or only inactive urinary sediment may remain after initiation of treatment.

The lupus nephritis can be any one of classes I-VI. For example, the lupus to be treated can be class II lupus nephritis, class III lupus nephritis, class IV lupus nephritis, or class V lupus nephritis.

In one embodiment, prior to initiation of treatment, the subject displays proteinuria greater than 500 mg per day, greater than 1000 mg per day, greater than 2000 mg per day, or greater than 3500 mg per day. In one embodiment, proteinuria is reduced after initiation of treatment with the antibody, for example, by at least 25%, by at least 50%, by at least 75%, or by at least 90%, or to less than 1 g per day or less than 500 mg per day. In one embodiment, protein to creatinine ratio is reduced after initiation of treatment with the antibody, e.g., by at least 25% or by at least 50%, or to less than 1 or less than 0.5.

The non-depleting CD4 antibody can be selected from the group consisting of: a) an antibody that comprises a light chain amino acid sequence set forth in SEQ ID NO:3 and a heavy chain amino acid sequence set forth in SEQ ID NO:6; b) an antibody that comprises a light chain amino acid sequence set forth in SEQ ID NO:9 and a heavy chain amino acid sequence set forth in SEQ ID NO:12; c) an antibody that comprises a light chain amino acid sequence set forth in SEQ ID NO:15 and a heavy chain amino acid sequence set forth in SEQ ID NO:18; d) an antibody that comprises a light chain amino acid sequence set forth in SEQ ID NO:21 and a heavy chain amino acid sequence set forth in SEQ ID NO:24; e) an antibody that comprises a CD4 binding fragment of the antibody of a), b), c), or d); f) an antibody that comprises CDR3 of the light chain shown in FIG. 1A (SEQ ID NO:27); g) an antibody that comprises CDR3 of the heavy chain shown in FIG. 1D (SEQ ID NO:30); h) an antibody that comprises CDR1, CDR2, and CDR3 of the light chain shown in FIG. 1A (SEQ ID NOs:25-27); i) an antibody that comprises CDR1, CDR2, and CDR3 of the heavy chain shown in FIG. 1D (SEQ ID NOs:28-30); and j) an antibody that comprises CDR1, CDR2, and CDR3 of the light chain shown in FIG. 1A and CDR1, CDR2, and CDR3 of the heavy chain shown in FIG. 1D (SEQ ID NOs:25-30). Similarly, the antibody can be a CD4 antibody that binds the same epitope as an antibody shown in any of FIGS. 1-4.

Essentially all of the features noted for the methods above apply to these embodiments as well, as relevant, for example with respect to optional combination of the non-depleting antibody with at least a second compound, type of antibody, and/or the like. For example, in an embodiment of the invention, the non-depleting CD4 antibody is optionally a humanized antibody, has an aglycosylated Fc portion, does not bind to the Fc receptor, includes amino acid substitutions altering C1 q binding and/or complement-dependent cytotoxicity, comprises a salvage receptor binding epitope, comprises a serum albumin binding peptide, and/or has three or more antigen-binding sites. In certain embodiments, the antibody is an anti-CD4 variant antibody that can bind a FcRN receptor.

Another general class of embodiments provides methods of treating lupus in a mammalian subject, e.g., a human. In the methods, a non-depleting CD4 antibody is administered to the subject in an amount effective to decrease titer of one or more autoantibodies (e.g., anti-double-stranded DNA antibodies). Generally, after initiation of treatment with the non-depleting CD4 antibody, the subject displays a reduction in the autoantibody titer or titers, as compared to the autoantibody titer or titers displayed by the subject prior to initiation of treatment. For example, the titer can be reduced by at least 25%, by at least 50%, by at least 75%, or by at least 90%. The methods optionally include determining the subject's autoantibody titer before and after initiation of treatment. Essentially all of the features noted for the methods above apply to these embodiments as well, as relevant.

A related general class of embodiments provides methods of decreasing autoantibody titer in a mammalian subject, e.g., a human. In the methods, a non-depleting CD4 antibody or a combination of a non-depleting CD4 antibody and at least a second compound (e.g., mycophenolate mofetil) is administered to the subject in an amount effective to decrease the autoantibody titer (e.g., anti-double-stranded DNA antibody titer). Generally, after initiation of treatment with the non-depleting antibody or the combination, the subject displays a reduction in autoantibody titer, as compared to the autoantibody titer displayed by the subject prior to initiation of treatment. For example, the titer can be reduced by at least 25%, by at least 50%, by at least 75%, or by at least 90%. The methods optionally include determining the subject's autoantibody titer before and after initiation of treatment. The subject typically has an autoimmune disorder or disease such as lupus. Essentially all of the features noted for the methods above apply to these embodiments as well, as relevant.

Yet another general class of embodiments provides methods of treating lupus in a mammalian subject, e.g., a human. In the methods, a therapeutically effective amount of a non-depleting CD4 antibody is administered to the subject. After initiation of treatment with the non-depleting antibody, the subject displays a reduction in anti-double-stranded DNA antibody titer (or other autoantibody titer), as compared to the anti-double-stranded DNA antibody titer (or other autoantibody titer) displayed by the subject prior to initiation of treatment. For example, the titer can be reduced by at least 25%, by at least 50%, by at least 75%, or by at least 90%. Essentially all of the features noted for the methods above apply to these embodiments as well, as relevant.

A related general class of embodiments provides methods of decreasing double-stranded DNA antibody or other autoantibody titer in a mammalian subject, e.g., a human. In the methods, a therapeutically effective amount of a non-depleting CD4 antibody or a therapeutically effective amount of a combination of a non-depleting CD4 antibody and at least a second compound (e.g., mycophenolate mofetil) is administered to the subject. After initiation of treatment with the non-depleting antibody or the combination, the subject displays a reduction in anti-double-stranded DNA antibody or other autoantibody titer, as compared to the anti-double-stranded DNA antibody or other autoantibody titer displayed by the subject prior to initiation of treatment. For example, the titer can be reduced by at least 25%, by at least 50%, by at least 75%, or by at least 90%. The methods optionally include determining the subject's double-stranded DNA antibody titer or other autoantibody titer before and after initiation of treatment. The subject typically has an autoimmune disorder or disease such as lupus. Essentially all of the features noted for the methods above apply to these embodiments as well, as relevant.

One general class of embodiments provides methods of treating multiple sclerosis in a mammalian subject, e.g., a human subject. In the methods, a therapeutically effective amount of a non-depleting CD4 antibody and/or at least a second compound is administered to the subject. For example, suitable second compounds include, but are not limited to, e.g., a cytotoxic agent; an immunosuppressive agent (e.g., cyclophosphamide); a B-cell surface marker antagonist; an antibody to a B-cell surface marker; a CD20 antibody (e.g., Rituximab); a CD5, CD28, or CD40 antibody or blocking agent; a corticosteroid (e.g., prednisone), CTLA4-Ig, an α4-integrin antibody such as natalizumab (Tysabri®), mycophenolate mofetil, a statin, an LFA-1 or CD-11a antibody or blocking agent, an interleukin-12 antibody, a beta interferon (e.g., an interferon β-1a such as Avonex® or Rebif®, or an interferon β-1b such as Betaseron®), glatiramer acetate (Copaxone®), a CD52 antibody such as alemtuzuman (CamPath®), an interleukin receptor antibody such as daclizumab (Zenapax®, an antibody to the interleukin-2 receptor alpha subunit), etc.

A related class of embodiments provides methods of treating a condition in a mammalian subject (e.g., a human subject). The condition can be rheumatoid arthritis, asthma, psoriasis, transplant rejection, graft versus host disease, multiple sclerosis, Crohn's disease, ulcerative colitis, Sjogren's syndrome, or another autoimmune disorder or disease. In the methods, a therapeutically effective amount of a combination of a non-depleting CD4 antibody and at least a second compound is administered to the subject. In one class of embodiments, the second compound is cyclophosphamide, mycophenolate mofetil, or CTLA4-Ig.

Essentially all of the features noted for the methods above apply to these classes of embodiments as well, as relevant, for example with respect to type of antibody, type of second compound, and/or the like. For example, the non-depleting CD4 antibody can be selected from the group consisting of: a) an antibody that comprises a light chain amino acid sequence set forth in SEQ ID NO:3 and a heavy chain amino acid sequence set forth in SEQ ID NO:6; b) an antibody that comprises a light chain amino acid sequence set forth in SEQ ID NO:9 and a heavy chain amino acid sequence set forth in SEQ ID NO:12; c) an antibody that comprises a light chain amino acid sequence set forth in SEQ ID NO:15 and a heavy chain amino acid sequence set forth in SEQ ID NO:18; d) an antibody that comprises a light chain amino acid sequence set forth in SEQ ID NO:21 and a heavy chain amino acid sequence set forth in SEQ ID NO:24; e) an antibody that comprises a CD4 binding fragment of the antibody of a), b), c), or d); f) an antibody that comprises CDR3 of the light chain shown in FIG. 1A (SEQ ID NO:27); g) an antibody that comprises CDR3 of the heavy chain shown in FIG. 1D (SEQ ID NO:30); h) an antibody that comprises CDR1, CDR2, and CDR3 of the light chain shown in FIG. 1A (SEQ ID NOs:25-27); i) an antibody that comprises CDR1, CDR2, and CDR3 of the heavy chain shown in FIG. 1D (SEQ ID NOs:28-30); and j) an antibody that comprises CDR1, CDR2, and CDR3 of the light chain shown in FIG. 1A and CDR1, CDR2, and CDR3 of the heavy chain shown in FIG. 1D (SEQ ID NOs:25-30). Similarly, the antibody can be a CD4 antibody that binds the same epitope as an antibody shown in any of FIGS. 1-4.

### BRIEF DESCRIPTION OF THE DRAWINGS

FIGS. 1A-1F show the nucleotide and amino acid sequences of the heavy and light chains of one embodiment of the TRX1 non-depleting CD4 antibody. FIG. 1A presents the nucleotide (SEQ ID NO:1) and amino acid (SEQ ID NO:2) sequences of the light chain, as well as the CDR and framework regions. FIG. 1B presents the nucleotide sequence of the light chain (SEQ ID NO:1). FIG. 1C presents the amino acid sequence of the light chain with (SEQ ID NO:2) and without (SEQ ID NO:3) the leader sequence. FIG. 1D presents the nucleotide (SEQ ID NO:4) and amino acid (SEQ ID NO:5) sequences of the heavy chain, as well as the CDR and framework regions. FIG. 1E presents the nucleotide sequence of the heavy chain (SEQ ID NO:4). FIG. 1F presents the amino acid sequence of the heavy chain with (SEQ ID NO:5) and without (SEQ ID NO:6) the leader sequence.

FIGS. 2A-2F show the nucleotide and amino acid sequences of the heavy and light chains of one embodiment of the TRX1 non-depleting CD4 antibody. FIG. 2A presents the nucleotide (SEQ ID NO:7) and amino acid (SEQ ID NO:8) sequences of the light chain, as well as the CDR and framework regions. FIG. 2B presents the nucleotide sequence of the light chain (SEQ ID NO:7). FIG. 2C presents the amino acid sequence of the light chain with (SEQ ID NO:8) and without (SEQ ID NO:9) the leader sequence. FIG. 2D presents the nucleotide (SEQ ID NO:10) and amino acid (SEQ ID NO:11) sequences of the heavy chain, as well as the CDR and framework regions. FIG. 2E presents the nucleotide sequence of the heavy chain (SEQ ID NO:10). FIG. 2F presents the amino acid sequence of the heavy chain with (SEQ ID NO:11) and without (SEQ ID NO:12) the leader sequence.

FIGS. 3A-3F show the nucleotide and amino acid sequences of the heavy and light chains of one embodiment of the TRX1 non-depleting CD4 antibody. FIG. 3A presents the nucleotide (SEQ ID NO:13) and amino acid (SEQ ID NO:14) sequences of the light chain, as well as the CDR and framework regions. FIG. 3B presents the nucleotide sequence of the light chain (SEQ ID NO:13). FIG. 3C presents the amino acid sequence of the light chain with (SEQ ID NO:14) and without (SEQ ID NO:15) the leader sequence. FIG. 3D presents the nucleotide (SEQ ID NO:16) and amino acid (SEQ ID NO:17) sequences of the heavy chain, as well as the CDR and framework regions. FIG. 3E presents the nucleotide sequence of the heavy chain (SEQ ID NO:16). FIG. 3F presents the amino acid sequence of the heavy chain with (SEQ ID NO:17) and without (SEQ ID NO:18) the leader sequence.

FIGS. 4A-4F show the nucleotide and amino acid sequences of the heavy and light chains of one embodiment of the TRX1 non-depleting CD4 antibody. FIG. 4A presents the nucleotide (SEQ ID NO:19) and amino acid (SEQ ID NO:20) sequences of the light chain, as well as the CDR and framework regions. FIG. 4B presents the nucleotide sequence of the light chain (SEQ ID NO:19). FIG. 4C presents the amino acid sequence of the light chain with (SEQ ID NO:20) and without (SEQ ID NO:21) the leader sequence. FIG. 4D presents the nucleotide (SEQ ID NO:22) and amino acid (SEQ ID NO:23) sequences of the heavy chain, as well as the CDR and framework regions. FIG. 4E presents the nucleotide sequence of the heavy chain (SEQ ID NO:22). FIG. 4F presents the amino acid sequence of the heavy chain with (SEQ ID NO:23) and without (SEQ ID NO:24) the leader sequence.

FIG. 5 schematically illustrates progression of disease by age in the NZBxW F1 preclinical efficacy model of SLE.

FIGS. 6A-6F present graphs illustrating response to administration of the non-depleting CD4 antibody. Graphs presented are time to progression (300 mg/dl proteinuria or death) in FIG. 6A, percent survival as a function of time after initiation of treatment in FIG. 6B, proteinuria at month 5 of treatment in FIG. 6C, and mean blood urea nitrogen as a function of time after initiation of treatment in FIG. 6D, for animals in which treatment was initiated at eight months of age. FIG. 6E shows time to progression (300 mg/dl proteinuria) and FIG. 6F shows percent survival as a function of time after initiation of treatment, for animals in which treatment was initiated at six months of age.

FIGS. 7A-7B present graphs illustrating reversal of severe lupus nephritis by treatment with the non-depleting CD4 antibody. FIG. 7A presents a graph showing the percentage of mice under 300 mg/dl proteinuria at the indicated times after treatment. FIG. 7B shows the percentage of mice reversed from ≧300 mg/dl proteinuria within the first month of treatment.

FIGS. 8A-8D present graphs illustrating response to administration of the non-depleting CD4 antibody. FIG. 8A shows ds-DNA antibody titer at enrollment, while FIG. 8B shows titer three months post-treatment. FIG. 8C shows the number of CD4+CD69+ cells found in spleen three weeks post-treatment. FIG. 8D shows the number of CD4+CD25+ cells found in spleen three weeks post-treatment.

FIGS. 9A-9B illustrate multiple comparison analysis of proteinuria at month 6 of treatment, using the cyclophosphamide (Cytoxan®) treated group as the control group in FIG. 9A and the CD4 non-depleting antibody treated group as the control group in FIG. 9B.

FIG. 10 schematically illustrates progression of disease over time in relapsing and remitting EAE induced by injection of PLP peptide in SJL/J mice, a preclinical efficacy model of MS.

FIGS. 11A-11B present graphs illustrating response to administration of the non-depleting CD4 antibody. FIG. 11A presents a graph of the clinical score over time for groups treated with the control antibody, glatiramer acetate (Copaxone®), the alpha-4 integrin antibody, CTLA4-Ig, and the non-depleting CD4 antibody. FIG. 11B presents the average daily clinical scores for these groups.

FIGS. 12A-12B present graphs illustrating response to administration of the non-depleting CD4 antibody. FIG. 12A presents a graph of the clinical score over time for groups treated with the control antibody, CTLA4-Ig, and the non-depleting CD4 antibody. FIG. 12B presents the average daily clinical scores for these groups.

FIGS. 13A-13B present graphs illustrating response to administration of the non-depleting CD4 antibody. FIG. 13A presents a graph of the clinical score over time for groups treated with the control antibody, CTLA4-Ig, and the non-depleting CD4 antibody. FIG. 13B presents the average daily clinical scores for these groups.

FIG. 14 depicts spinal cord sections from mice treated with the control antibody or the CD4 antibody, showing that non-depleting CD4 antibody treatment decreases demyelination in EAE.

FIG. 15 presents graphs showing the number of ICOS^{hi}CD4 or ICOS^{hi}CD8 T cells per μl of blood for animals treated with the control antibody, the non-depleting CD4 antibody, or CTLA4-Ig.

FIG. 16 presents a graph of the clinical score over time comparing treatment of myelin oligodendrocyte glycoprotein (MOG)-peptide induced EAE with a non-depleting CD4 antibody, a depleting CD4 antibody, a control antibody, CTLA4-Ig, or a depleting CD8 antibody.

FIGS. 17A-17B present graphs illustrating response to administration of the non-depleting CD4 antibody. FIG. 17A presents a graph showing the percentage of mice under 300 mg/dl proteinuria at the indicated times after indicated treatment. FIG. 17B shows the percentage of mice reversed from ≧300 mg/dl proteinuria.

FIGS. 18A-18D present graphs illustrating response to treatment. Graphs presented illustrate time to progression (300 mg/dl proteinuria or death) in FIG. 18A and percent survival as a function of time after initiation of treatment in FIG. 18B for animals treated with a combination of non-depleting CD4 antibody and 50 mg/kg per day MMF, and time to progression in FIG. 18C and percent survival in FIG. 18D for animals treated with a combination of non-depleting CD4 antibody and 25 mg/kg per day MMF.

FIGS. 19A-19B illustrate multiple comparison analysis of proteinuria at month 2 of treatment, using the control antibody treated group as the control group. Results for groups treated with 50 mg/kg of MMF daily (alone or in combination with non-depleting CD4 antibody) are presented in FIG. 19A, while results for groups treated with 25 mg/kg of MMF daily are presented in FIG. 19B.

FIGS. 20A-20I present graphs illustrating response to treatment. Graphs presented show the number of CD4⁺ T cells per μl of blood (FIG. 20A), B2 B cells per μl of blood (FIG. 20B), CD4⁺ T cells per spleen (FIG. 20C), B2 B cells per spleen (FIG. 20D), IgM+ plasma cells (FIG. 20E), isotype-switched plasma cells (FIG. 20F), germinal center cells (FIG. 20G), and plasmacytoid dendritic cells per spleen (FIG. 20H), and MHC II expression level in plasmacytoid dendritic cells (FIG. 20I), for animals treated with the control antibody, the non-depleting CD4 antibody, the indicated dose of MMF, or a combination of the non-depleting CD4 antibody and the indicated dose of MMF.

FIGS. 21A-21B show the nucleic acid and amino acid sequences of human CD4. FIG. 21A presents the human CD4 amino acid sequence for mature protein with leader cleaved. FIG. 21B presents the mature human CD4 DNA sequence.

FIG. 22 shows serum anti-dsDNA antibody levels following two months of treatment with the indicated agents. The average baseline titers at treatment onset are indicated by the dotted line.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Any methods and materials similar or equivalent to those described herein can be used in the practice for testing of the present invention, and non-limiting materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

“Lupus” as used herein is an autoimmune disease or disorder involving antibodies that attack connective tissue. The principal form of lupus is a systemic one, systemic lupus erythematosus (SLE), which may include cutaneous involvement. “Lupus” as used herein includes SLE as well as other types of lupus (including, e.g., cutaneous lupus erythematosus (CLE), lupus nephritis (LN), extrarenal, cerebritis, pediatric, non-renal, discoid, and alopecia).

A “subject” herein is typically a human, but can be a non-human mammal. Exemplary non-human mammals include laboratory, domestic, pet, sport, and stock animals, e.g., mice, cats, dogs, horses, and cows. Typically, the subject is eligible for treatment, e.g., treatment of an autoimmune disorder, treatment related to a tissue transplant, or the like. In one aspect, such subject is eligible for treatment for lupus. For the purposes herein, such eligible subject is one that is experiencing or has experienced one or more signs, symptoms, or other indicators of lupus or has been diagnosed with lupus, whether, for example, newly diagnosed, previously diagnosed with a new flare, or chronically steroid dependent with a new flare, or is at risk for developing lupus. One eligible for treatment of lupus may optionally be identified as one who is screened by renal biopsy and/or is screened using an assay to detect auto-antibodies, such as those noted below, wherein autoantibody production is assessed qualitatively, and preferably quantitatively. Exemplary such auto-antibodies associated with SLE are anti-nuclear antibodies (Ab), anti-double-stranded DNA (dsDNA) Ab, anti-Sm Ab, anti-nuclear ribonucleoprotein Ab, anti-phospholipid Ab, anti-ribosomal P Ab, anti-Ro/SS-A Ab, anti-Ro Ab, and anti-La Ab.

Diagnosis of lupus (and determination of eligibility for treatment) can be performed as established in the art. For example, diagnosis of SLE may be according to current American College of Rheumatology (ACR) criteria. Active disease may be defined by one British Isles Lupus Activity Group's (BILAG) “A” criteria or two BILAG “B” criteria, e.g., as applied in U.S. patent application publication 2006/0024295 by Brunetta entitled “Method for treating lupus.” Some signs, symptoms, or other indicators used to diagnose SLE adapted from Tan et al. (1982) “The 1982 Revised Criteria for the Classification of SLE” Arth Rheum 25:1271-1277 may be malar rash such as rash over the cheeks, discoid rash, or red raised patches, photosensitivity such as reaction to sunlight, resulting in the development of or increase in skin rash, oral ulcers such as ulcers in the nose or mouth, usually painless, arthritis, such as non-erosive arthritis involving two or more peripheral joints (arthritis in which the bones around the joints do not become destroyed), serositis, pleuritis or pericarditis, renal disorder such as excessive protein in the urine (proteinuria, greater than 0.5 g (gram)/day or 3+ on test sticks) and/or cellular casts (abnormal elements derived from the urine and/or white cells and/or kidney tubule cells), neurologic signs, symptoms, or other indicators, seizures (convulsions), and/or psychosis in the absence of drugs or metabolic disturbances that are known to cause such effects, and hematologic signs, symptoms, or other indicators such as hemolytic anemia or leukopenia (white bloodcount below 4,000 cells per cubic millimeter) or lymphopenia (less than 1,500 lymphocytes per cubic millimeter) or thrombocytopenia (less than 100,000 platelets per cubic millimeter). The leukopenia and lymphopenia must be detected on two or more occasions. The thrombocytopenia must be detected in the absence of drugs known to induce it. The invention is not limited to these signs, symptoms, or other indicators of lupus.

A nephritic lupus flare can be defined as 1) an increase of >30% in Scr within a 1-month period, or 2) a recurrence or appearance of nephrotic syndrome, or 3) a 3-fold increase in urinary protein with baseline proteinuria >1 g/24 hrs or as noted in U.S. patent application publication 2006/0024295. For lupus nephritis, the treatment eligibility may be evidenced by a nephritic flare as defined by renal criteria as noted in U.S. patent application publication 2006/0024295.

Lupus nephritis is optionally diagnosed and classified as ISN/WHO class I, class II, class III, class IV, class V, or class VI lupus nephritis, e.g., as set forth in Weening et al. (2004) “The classification of glomerulonephritis in systemic lupus erythematosus revisited” Kidney International 65:521-530.

“Treatment” of a subject herein refers to both therapeutic treatment and prophylactic or preventative measures. Those in need of treatment include those already with lupus (or another condition or autoimmune disorder such as MS, rheumatoid arthritis, or inflammatory bowel disease) as well as those in which the lupus (or other disorder) is to be prevented. Hence, the subject may have been diagnosed as having lupus (or another disorder) or may be predisposed or susceptible to the lupus (or other disorder).

The term “ameliorates” or “amelioration” as used herein refers to a decrease, reduction or elimination of a condition, disease, disorder, or phenotype, including an abnormality or symptom.

A “symptom” of a disease or disorder (e.g., lupus) is any morbid phenomenon or departure from the normal in structure, function, or sensation, experienced by a subject and indicative of disease.

The expression “therapeutically effective amount” refers to an amount that is effective for preventing, ameliorating, or treating a disease or disorder (e.g., lupus, MS, rheumatoid arthritis, or inflammatory bowel disease). For example, a “therapeutically effective amount” of an antibody refers to an amount of the antibody that is effective for preventing, ameliorating, or treating the specified disease or disorder. Similarly, a “therapeutically effective amount” of a combination of an antibody and a second compound refers to an amount of the antibody and an amount of the second compound that, in combination, are effective for preventing, ameliorating, or treating the specified disease or disorder.

It is to be understood that the terminology “a combination of” two compounds does not mean that the compounds have to be administered in admixture with each other. Thus, treatment with or use of such a combination encompasses a mixture of the compounds or separate administration of the compounds, and includes administration on the same day or different days. Thus the terminology “combination” means two or more compounds are used for the treatment, either individually or in admixture with each other. When an antibody and a second compound, for example, are administered in combination to a subject, the antibody is present in the subject at a time when the second compound is also present in the subject, whether the antibody and second compound are administered individually or in admixture to the subject.

The CD4 antigen, or “CD4,” is a glycoprotein expressed on the surface of T lymphocytes, as well as certain other cells. Other names for CD4 in the literature include cluster of differentiation 4 and L3T4. CD4 is described, for example, in entry 186940 in the Online Mendelian Inheritance in Man database, on the world wide web at www (dot) ncbi (dot) nlm (dot) nih (dot) gov/Omim.

A “CD4 antibody” is an antibody that binds CD4 with sufficient affinity and specificity. For example, the antibody optionally binds CD4 with an affinity and specificity for CD4 that are comparable to or substantially similar to the binding affinity and specificity of a TRX1 antibody for CD4. As used herein, a “CD4 antibody,” an “anti-CD4 antibody,” and an “anti-CD4” are equivalent terms and are used interchangeably.

A “non-depleting CD4 antibody” is a CD4 antibody that depletes less than 50% of CD4+ cells, preferably less than 25% of CD4+ cells, and most preferably less than 10% of CD4+ cells. Conversely, a “depleting CD4 antibody” is a CD4 antibody that depletes 50% or more of CD4+ cells, or even 75% or more or 90% or more of CD4+ cells. Depletion of CD4+ cells (e.g., reduction in circulating CD4+ cell levels in a subject treated with the antibody) can be achieved by various mechanisms, such as antibody-dependent cell-mediated cytotoxicity, complement-dependent cytotoxicity, inhibition of T-cell proliferation, and/or induction of T-cell death.

The term “antibody” herein is used in the broadest sense and specifically covers monoclonal antibodies, polyclonal antibodies, multispecific antibodies (e.g. bispecific antibodies) formed from at least two intact antibodies, chimeric antibodies, human antibodies, and antibody fragments so long as they exhibit the desired biological activity (e.g., CD4 binding). An antibody is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes.

“Antibody fragments” comprise a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments.

An “intact antibody” is one comprising heavy- and light-variable domains as well as an Fc region.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light-chain and the heavy-chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al. Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cell-mediated cytotoxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy-chain and one light-chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy-chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known. See, e.g., Fundamental Immunology, W. E. Paul, ed., Raven Press, N.Y. (1999), for a more detailed description of other antibody fragments.

While various antibody fragments are defined in terms of the digestion of an intact antibody, one of skill will appreciate that such fragments may be synthesized de novo either chemically or by utilizing recombinant DNA methodology. Thus, the term antibody, as used herein, includes antibodies or fragments thereof either produced by the modification of whole antibodies or synthesized de novo using recombinant DNA methodologies.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μA, respectively. The subunit structures and three-dimensional configurations of different classes of immunoglobulins are well known.

“Single-chain Fv” or “scFv” antibody fragments that comprise the V_{H} and V_{L} domains of antibody, wherein these domains are present in a single polypeptide chain. Preferably, the Fv polypeptide further comprises a polypeptide linker between the V_{H} and V_{L} domains that enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 1993/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable-domain antigen-binding sequences derived from a non-human primate (e.g. Old World Monkey, such as baboon, rhesus, or cynomolgus monkey) and human constant-region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody that are responsible for antigen binding. The hypervariable region comprises amino acid residues from a “complementarity-determining region” or “CDR” (see, e.g., Kabat et al. Sequences of Proteins of Immunological Interest, 5th Ed. Public Health Service, National Institutes of Health, Bethesda, Md. (1991)) and/or those residues from a “hypervariable loop” (see, e.g., Chothia and Lesk J. Mol. Biol. 196:901-917 (1987)). “Framework” or “FR” residues are those variable-domain residues other than the hypervariable region residues as herein defined.

The terms “Fc receptor” and “FcR” are used to describe a receptor that binds to the Fc region of an antibody. FcRs are reviewed in Ravetch and Kinet (1991) Annu. Rev. Immunol 9:457-92; Capel et al. (1994) Immunomethods 4:25-34; and de Haas et al. (1995) J. Lab. Clin. Med. 126:330-41. Other FcRs, including those to be identified in the future, are encompassed by the term “FcR” herein. The term also includes the neonatal receptor, FcRn, which is responsible for the transfer of maternal IgGs to the fetus (Guyer et al. (1976) J. Immunol. 117:587 and Kim et al. (1994) J. Immunol. 24:249).

A “CD4 binding fragment” of an antibody is a fragment of the antibody that retains the ability to bind CD4. As noted, the fragment is optionally produced by digestion of the intact antibody or synthesized de novo.

An “epitope” is the specific region of an antigenic molecule that binds to an antibody.

The phrase “substantially similar,” or “substantially the same”, as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristic measured by said values (e.g., Kd values). The difference between said two values is preferably less than about 50%, preferably less than about 40%, preferably less than about 30%, preferably less than about 20%, preferably less than about 10% as a function of the value for the reference/comparator antibody.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

In one embodiment, the “Kd” or “Kd value” according to this invention is measured by a radiolabeled antigen binding assay (RIA) performed with the Fab version of an antibody of interest and its antigen as described by the following assay that measures solution binding affinity of Fabs for antigen by equilibrating Fab with a minimal concentration of [125I]-labeled antigen in the presence of a titration series of unlabeled antigen, then capturing bound antigen with an anti-Fab antibody-coated plate (Chen et al. (1999) J. Mol Biol 293:865-881). To establish conditions for the assay, microtiter plates (Dynex) are coated overnight with 5 ug/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbant plate (Nunc #269620), 100 pM or 26 pM [125I]-antigen are mixed with serial dilutions of a Fab of interest (e.g., consistent with assessment of an anti-VEGF antibody, Fab-12, in Presta et al. (1997) Cancer Res. 57:4593-4599). The Fab of interest is then incubated overnight; however, the incubation may continue for a longer period (e.g., 65 hours) to insure that equilibrium is reached. Thereafter, the mixtures are transferred to the capture plate for incubation at room temperature (e.g., for one hour). The solution is then removed and the plate washed eight times with 0.1% Tween-20 in PBS. When the plates have dried, 150 ul/well of scintillant (MicroScint™-20; Packard) is added, and the plates are counted on a Topcount® gamma counter (Packard) for ten minutes. Concentrations of each Fab that give less than or equal to 20% of maximal binding are chosen for use in competitive binding assays. According to another embodiment, the Kd or Kd value is measured by using surface plasmon resonance assays using a BIAcore®-2000 or a BIAcore®-3000 (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 ug/ml (˜0.2 uM) before injection at a flow rate of 5 ul/minute to achieve approximately 10 response units (RU) of coupled protein. Following the injection of antigen, 1M ethanolamine is injected to block unreacted groups. For kinetics measurements, two-fold serial dilutions of Fab (0.78 nM to 500 nM) are injected in PBS with 0.05% Tween 20 (PBST) at 25° C. at a flow rate of approximately 25 ul/min. Association rates (kon) and dissociation rates (koff) are calculated using a simple one-to-one Langmuir binding model (BIAcore® Evaluation Software version 3.2) by simultaneously fitting the association and dissociation sensorgram. The equilibrium dissociation constant (Kd) is calculated as the ratio koff/kon. See, e.g., Chen et al. (1999) J. Mol Biol 293:865-881. If the on-rate exceeds 10⁶ M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-Aminco® spectrophotometer (ThermoSpectronic) with a stirred cuvette.

An “amino acid sequence” is a polymer of amino acid residues (a protein, polypeptide, etc.) or a character string representing an amino acid polymer, depending on context.

The term “immunosuppressive agent” as used herein for therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); nonsteroidal antiinflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); hydroxycloroquine; sulfasalazine; leflunomide; cytokine or cytokine receptor antibodies including anti-interferon-alpha, -beta, or -gamma antibodies, anti-tumor necrosis factor-alpha antibodies (infliximab or adalimumab), anti-TNF-alpha immunoahesin (etanercept), anti-tumor necrosis factor-beta antibodies, anti-interleukin-2 antibodies and anti-IL-2 receptor antibodies; anti-LFA-1 antibodies, including anti-CD11a and anti-CD18 antibodies; heterologous anti-lymphocyte globulin; pan-T antibodies, preferably anti-CD3; soluble peptide containing a LFA-3 binding domain (WO 1990/08187 published Jul. 26, 1990); streptokinase; TGF-beta; streptodornase; RNA or DNA from the host; FK506; RS-61443; deoxyspergualin; rapamycin; T-cell receptor (Cohen et al., U.S. Pat. No. 5,114,721); T-cell-receptor fragments (Offner et al., Science, 251: 430-432 (1991); WO 1990/11294; Ianeway, Nature, 341: 482 (1989); and WO 1991/01133); and T-cell-receptor antibodies (EP 340,109) such as T10B9.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small-molecule toxins or enzymatically active toxins of bacterial, fungal, plant, or animal origin, or fragments thereof.

A “chemotherapeutic agent” is a chemical compound typically useful in the treatment of cancer. Examples of chemotherapeutic agents include alkylating agents such as thiotepa and CYTOXAN® cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphorami-de, and trimethylolomelamine; acetogenins (especially bullatacin and bullatacinone); a camptothecin (including the synthetic analogue topotecan); bryostatin; callystatin; CC-1065 (including its adozelesin, carzelesin, and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXAN™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids, or derivatives of any of the above.

Also included in this definition are anti-hormonal agents that act to regulate or inhibit hormone action such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LYI17018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, and H-Ras; vaccines such as gene-therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids, or derivatives of any of the above.

The term “cytokine” is a generic term for proteins released by one cell population that act on another cell as intercellular mediators. Examples of such cytokines are lymphokines, monokines; interleukins (ILs) such as IL-1, IL-1a, IL-2, IL-3, IL-4, IL-5, IL-6, IL-7, IL-8, IL-9, IL-11, IL-12, IL-15; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

The term “hormone” refers to polypeptide hormones, which are generally secreted by glandular organs with ducts. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle-stimulating hormone (FSH), thyroid-stimulating hormone (TSH), and luteinizing hormone (LH); prolactin, placental lactogen, mouse gonadotropin-associated peptide, inhibin; activin; mullerian-inhibiting substance; and thrombopoietin. As used herein, the term hormone includes proteins from natural sources or from recombinant cell culture and biologically active equivalents of the native-sequence hormone, including synthetically produced small-molecule entities and pharmaceutically acceptable derivatives and salts thereof.

The term “growth factor” refers to proteins that promote growth, and include, for example, hepatic growth factor; fibroblast growth factor; vascular endothelial growth factor; nerve growth factors such as NGF-β; platelet-derived growth factor; transforming growth factors (TGFs) such as TGF-α and TGF-β; insulin-like growth factor-I and -II; erythropoietin (EPO); osteoinductive factors; interferons such as interferon-α, β, and γ; and colony-stimulating factors (CSFs) such as macrophage-CSF (M-CSF); granulocyte-macrophage-CSF (GM-CSF); and granulocyte-CSF (G-CSF). As used herein, the term growth factor includes proteins from natural sources or from recombinant cell culture and biologically active equivalents of the native-sequence growth factor, including synthetically produced small-molecule entities and pharmaceutically acceptable derivatives and salts thereof.

For the purposes herein, “tumor necrosis factor-alpha (TNF-alpha)” refers to a human TNF-alpha molecule comprising the amino acid sequence as described in Pennica et al., Nature, 312:721 (1984) or Aggarwal et al., JBC, 260:2345 (1985).

A “TNF-alpha inhibitor” herein is an agent that inhibits, to some extent, a biological function of TNF-alpha, generally through binding to TNF-alpha and neutralizing its activity. Examples of TNF inhibitors specifically contemplated herein are etanercept (ENBREL®), infliximab (REMICADE®), and adalimumab (HUMIRA™).

Examples of “nonsteroidal anti-inflammatory drugs” or “NSAIDs” are acetylsalicylic acid, ibuprofen, naproxen, indomethacin, sulindac, tolmetin, including salts and derivatives thereof, etc.

The term “integrin” refers to a receptor protein that allows cells both to bind to and to respond to the extracellular matrix and is involved in a variety of cellular functions such as wound healing, cell differentiation, homing of tumor cells, and apoptosis. They are part of a large family of cell adhesion receptors that are involved in cell-extracellular matrix and cell-cell interactions. Functional integrins consist of two transmembrane glycoprotein subunits, called alpha and beta, that are non-covalently bound. The alpha subunits all share some homology to each other, as do the beta subunits. The receptors always contain one alpha chain and one beta chain. Examples include α6β1, α3β1, α7β1, LFA-1 etc. As used herein, the term integrin includes proteins from natural sources or from recombinant cell culture and biologically active equivalents of the native-sequence integrin, including synthetically produced small-molecule entities and pharmaceutically acceptable derivatives and salts thereof. An “α4-integrin” is the α4 subunit of α4-β1 and α4-β7 integrins that are expressed on the surface of leukocytes other than neutrophils.

Examples of “integrin antagonists or antibodies” herein include an LFA-1 antibody, such as efalizumab (RAPTIVA®) commercially available from Genentech, or an alpha 4 integrin antibody (e.g., a “α4-integrin antibody” is an antibody that binds α4-integrin) such as natalizumab (Tysabri®) available from Biogen, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. Nos. 6,677,339 and 6,348,463), aromatic amine derivatives (U.S. Pat. No. 6,369,229), ADAM disintegrin domain polypeptides (US 2002/0042368), antibodies to alphavbeta3 integrin (EP 633945), aza-bridged bicyclic amino acid derivatives (WO 2002/02556), etc.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone), dexamethasone triamcinolone, and betamethasone.

A “B-cell surface marker” or “B-cell surface antigen” herein is an antigen expressed on the surface of a B cell that can be targeted with an antagonist that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85, and CD86 leukocyte surface markers (for descriptions, see The Leukocyte Antigen Facts Book, 2nd Edition. 1997, ed. Barclay et al. Academic Press, Harcourt Brace & Co., New York). Other B-cell surface markers include RP105, FcRH2, B-cell CR2, CCR6, P2X5, HLA-DOB, CXCR5, FCER2, BR3, Btig, NAG14, SLGC16270, FcRH1, IRTA2, ATWD578, FcRH3, IRTA1, FcRH6, BCMA, and 239287. The B-cell surface marker of particular interest is preferentially expressed on B cells compared to other non-B-cell tissues of a mammal and may be expressed on both precursor B cells and mature B cells.

An “antibody that binds to a B-cell surface marker” is a molecule that, upon binding to a B-cell surface marker, destroys or depletes B cells in a mammal and/or interferes with one or more B-cell functions, e.g. by reducing or preventing a humoral response elicited by the B cell. The antibody preferably is able to deplete B cells (i.e. reduce circulating B-cell levels) in a mammal treated therewith. Such depletion may be achieved via various mechanisms such as antibody-dependent cell-mediated cytotoxicity (ADCC) and/or complement-dependent cytotoxicity (CDC), inhibition of B-cell proliferation, and/or induction of B-cell death (e.g. via apoptosis).

An “antagonist” refers to a molecule capable of neutralizing, blocking, inhibiting, abrogating, reducing or interfering with the activities of a particular or specified protein, including its binding to one or more receptors in the case of a ligand or binding to one or more ligands in case of a receptor. Antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include small molecule inhibitors of the protein, and fusion proteins, receptor molecules and derivatives which bind specifically to the protein thereby sequestering its binding to its target, antagonist variants of the protein, antisense molecules directed to the protein, RNA aptamers, and ribozymes against the protein.

A “B-cell surface marker antagonist” is a molecule that, upon binding to a B-cell surface marker, destroys or depletes B cells in a mammal and/or interferes with one or more B-cell functions, e.g. by reducing or preventing a humoral response elicited by the B cell. The antagonist preferably is able to deplete B cells (i.e. reduce circulating B-cell levels) in a mammal treated therewith. Such depletion may be achieved via various mechanisms such as ADCC and/or CDC, inhibition of B-cell proliferation, and/or induction of B-cell death (e.g. via apoptosis). Antagonists included within the scope of the present invention include antibodies, synthetic or native-sequence peptides, fusion proteins, and small-molecule antagonists that bind to the B-cell marker, optionally conjugated with or fused to a cytotoxic agent. Examples include but are not limited to, e.g., CD20 antibodies, BR3 antibodies (e.g., WO0224909), BR3-Fc, etc.

Examples of CD20 antibodies include: “C2B8,” which is now called “rituximab” (“RITUXAN®”) (U.S. Pat. No. 5,736,137); the yttrium-[90]-labeled 2B8 murine antibody designated “Y2B8” or “Ibritumomab Tiuxetan” (ZEVALIN®) commercially available from IDEC Pharmaceuticals, Inc. (U.S. Pat. No. 5,736,137; 2B8 deposited with ATCC under accession no. HB11388 on Jun. 22, 1993); murine IgG2a “B1,” also called “Tositumomab,” optionally labeled with ¹³¹I to generate the “¹³¹I-B1” or “iodine I¹³¹ tositumomab” antibody (BEXXAR™) commercially available from Corixa (see, also, U.S. Pat. No. 5,595,721); murine monoclonal antibody “1F5” (Press et al. Blood 69(2):584-591 (1987) and variants thereof including “framework-patched” or humanized 1F5 (WO 2003/002607, Leung, S.; ATCC deposit HB-96450); murine 2H7 and chimeric 2H7 antibody (U.S. Pat. No. 5,677,180); humanized 2H7 (see, e.g., WO04/056312; US20060024295); HUMAX-CD20™ antibodies (Genmab, Denmark); the human monoclonal antibodies set forth in WO 2004/035607 (Teeling et al.); AME-133™ antibodies (Applied Molecular Evolution); A20 antibody or variants thereof such as chimeric or humanized A20 antibody (cA20, hA20, respectively) (US 2003/0219433, Immunomedics); and monoclonal antibodies L27, G28-2, 93-1 B3, B-C1 or NU-B2 available from the International Leukocyte Typing Workshop (Valentine et al., In: Leukocyte Typing III (McMichael, Ed., p. 440, Oxford University Press (1987)).

Examples of “disease-modifying anti-rheumatic drugs” or “DMARDs” include hydroxycloroquine, sulfasalazine, methotrexate, leflunomide, etanercept, infliximab (plus oral and subcutaneous methrotrexate), azathioprine, D-penicillamine, Gold (oral), Gold (intramuscular), minocycline, cyclosporine, Staphylococcal protein A immunoadsorption, including salts and derivatives thereof, etc.

“CTLA4” is expressed on activated T lymphocytes and is involved in down-regulation of the immune response. Other names for CTLA4 in the literature include cytotoxic T-lymphocyte-associated antigen 4, cytotoxic T-lymphocyte-associated protein 4, cell differentiation antigen CD152, and cytotoxic T-lymphocyte-associated granule serine protease 4.

A variety of additional terms are defined or otherwise characterized herein.

### DETAILED DESCRIPTION

CD4 is a surface glycoprotein primarily expressed on cells of the T lymphocyte lineage, including a majority of thymocytes and a subset of peripheral T cells. Low levels of CD4 are also expressed by some non-lymphoid cells, although the functional significance of such divergent cellular distribution is unknown. On mature T cells, CD4 serves a co-recognition function through interaction with MHC Class II molecules expressed in antigen presenting cells. CD4+ T cells constitute primarily the helper subset which regulates T and B cell functions during T-dependent responses to viral, bacterial, fungal and parasitic infections.

During the pathogenesis of autoimmune diseases, in particular when tolerance to self antigens breaks down, CD4+ T cells can contribute to inflammatory responses which result in joint and tissue destruction. These processes are facilitated, e.g., by the recruitment of inflammatory cells of the hematopoietic lineage, production of antibodies, inflammatory cytokines and mediators, and by the activation of killer cells.

CD4+ T cells have been implicated in the pathogenesis of lupus. For example, CD4+ T cells are present in sites of glomerulonephritis. CD4+ T cells from SLE patients are reported to be hyper-responsive to antigen and resistant to apoptosis in vitro. Autoantigen-specific CD4+ T cells that can support production of autoantibodies by B cells (effector/memory CD4+ cells that produce IFN-γ) are present in SLE patients. In addition, a strong association between MHC Class II alleles and risk for SLE is observed.

CD4+ T cells have been similarly implicated in the pathogenesis of MS. For example, CD4+ helper T cells are involved in the pathogenesis of MS and a corresponding laboratory model, experimental allergic encephalomyelitis (EAE), and laboratory animals depleted of T cells exhibit a loss of ability to develop EAE (U.S. Pat. No. 4,695,459 to Steinman et al. entitled “Method of treating autoimmune diseases that are mediated by Leu3/CD4 phenotype T cells”, Traugott et al. (1983) “Multiple sclerosis: distribution of T cell subsets within active chronic lesions” Science 219:308-310, Amason et al. (1962) “Role of the thymus in immune reaction in rats: II. Suppressive effect of thymectomy at birth on reactions of delayed (cellular) hypersensitivity and the circulating small lymphocyte” J Exp Med 116:177-186, and Gonatas and Howard (1974) “Inhibition of experimental allergic encephalomyelitis in rats severely depleted of T cells” Science 186:839-841). CD4+ and CD8+ T cells are found in MS lesions; both are known to produce inflammatory cytokines, although their relative contribution to pathogenesis has not been determined. A four-fold increase is observed in the frequency of myelin-specific CD4+ cells in blood of MS patients. Several drugs currently used or which mostly will be used for treatment of MS are believed to work, in part, through their action on T cells; for example, Tysabri® (natalizumab, alpha-4 integrin antibody), CamPath® (alemtuzumab, CD52 antibody), and daclizumab (IL-2Rα antibody). In addition, an increased risk of MS is associated with MHC Class II alleles (3.6 fold) and, to a lesser extent, Class I alleles (2 fold).

In one aspect, the present invention provides methods of treating lupus, including SLE and lupus nephritis, by administering a combination of a non-depleting CD4 antibody and another compound used clinically or experimentally to treat lupus. Another aspect of the invention provides methods of treating lupus nephritis, including mid- to late-stage disease, by administration of a non-depleting CD4 antibody that results in an improvement in renal function and/or a reduction in proteinuria or active urinary sediment. Yet another aspect of the invention provides methods of treating MS by administration of a non-depleting CD4 antibody, optionally in combination with another compound used clinically or experimentally to treat MS. Yet another aspect of the invention provides methods of treating transplant recipients or subjects with autoimmune diseases such as rheumatoid arthritis, asthma, psoriasis, inflammatory bowel disease (e.g., Crohn's disease and ulcerative colitis), and Sjogren's syndrome by administration of a non-depleting CD4 antibody, typically in combination with another compound used clinically or experimentally to treat autoimmune disease.

### CD4 Antibodies

A number of CD4 antibodies, both depleting and non-depleting, have been described. Use of such antibodies to induce tolerance to antigens, including autoantigens, has also been reported. See, e.g., U.S. Pat. No. 4,695,459; U.S. Pat. No. 6,056,956 to Cobbold and Waldmann entitled “Non-depleting anti-CD4 monoclonal antibodies and tolerance induction”; U.S. Pat. No. 5,690,933 to Cobbold and Waldmann entitled “Monoclonal antibodies for inducing tolerance”; European patent application publication 0240344 by Cobbold et al. entitled “Monoclonal antibodies and their use”; U.S. Pat. No. 6,136,310 to Hanna et al. entitled “Recombinant anti-CD4 antibodies for human therapy”; U.S. Pat. No. 5,756,096 to Newman et al. entitled “Recombinant antibodies for human therapy”; U.S. Pat. No. 5,750,105 to Newman et al. entitled “Recombinant antibodies for human therapy”; U.S. Pat. No. 4,381,295 to Kung and Goldstein entitled “Monoclonal antibody to human helper T cells and methods of preparing same”; Waldmann (1989) “Manipulation of T-cell responses with monoclonal antibodies” Ann Rev Immunol 7:407-44; and Wofsy and Seaman (1987) “Reversal of advanced murine lupus in NZB/NZW F1 mice by treatment with monoclonal antibody to L3T4” J Immunol 138:3247-53. In particular, a non-depleting CD4 antibody and its use in inducing tolerance has been described in U.S. patent application publication 2003/0108518 by Frewin et al. entitled “TRX1 antibody and uses therefor” and U.S. patent application publication 2003/0219403 by Frewin et al. entitled “Compositions and methods of tolerizing a primate to an antigen”, each of which is hereby incorporated by reference.

Exemplary non-depleting CD4 antibodies suitable for use in the methods include the TRX1 antibodies described in U.S. patent application publication 2003/0108518 by Frewin et al. entitled “TRX1 antibody and uses therefor” and U.S. patent application publication 2003/0219403 by Frewin et al. entitled “Compositions and methods of tolerizing a primate to an antigen.” These antibodies are humanized antibodies including modified constant regions of a human antibody, light and heavy chain framework regions of a human antibody, and light and heavy chain CDRs derived from a mouse monoclonal antibody.

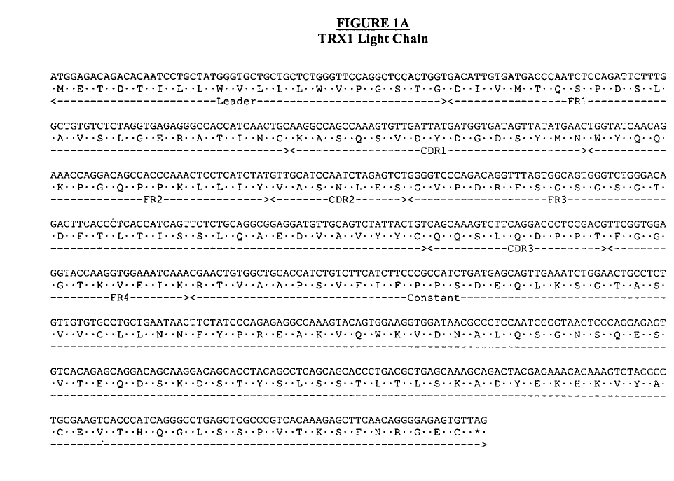

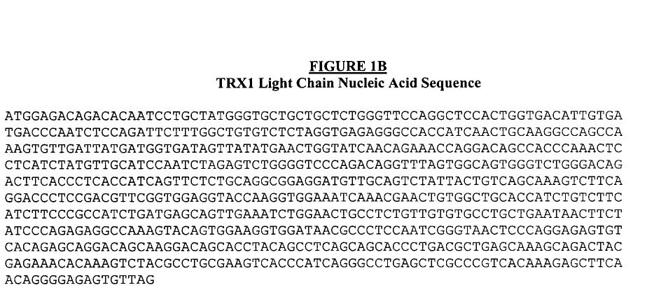

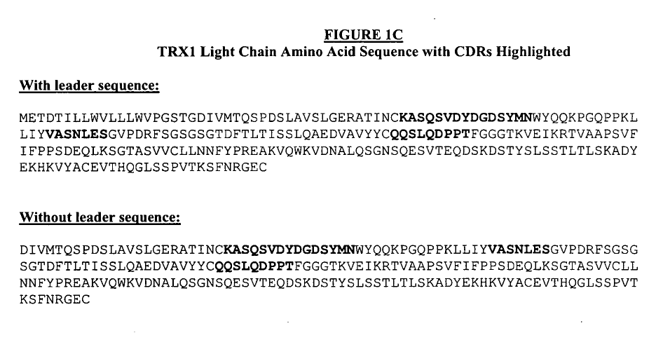

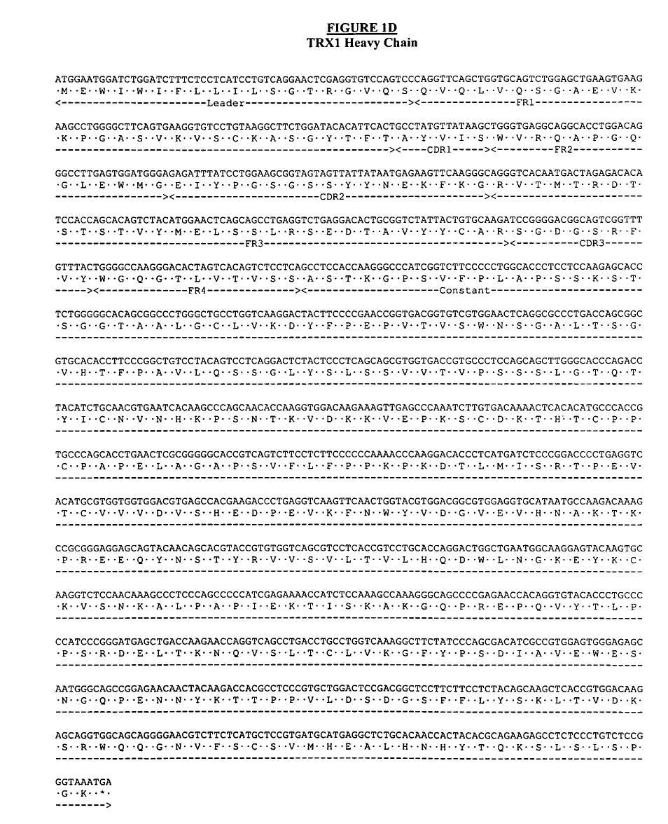

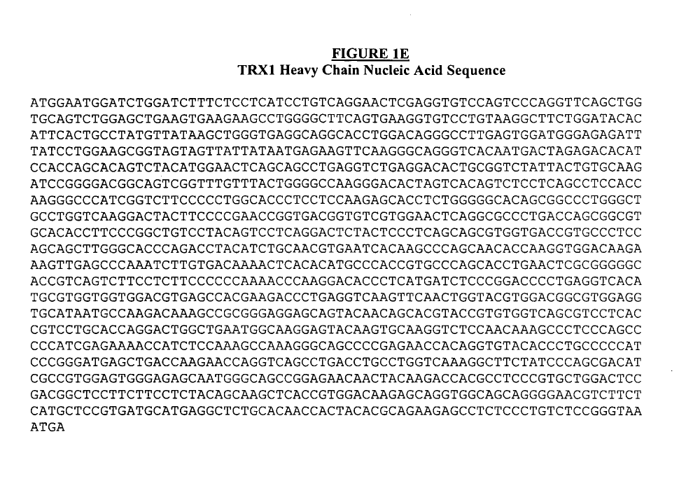

Thus, in one class of embodiments, the non-depleting CD4 antibody is a TRX1 antibody as shown in any one of FIGS. 1-4. The antibody can have a light chain amino acid sequence set forth in SEQ ID NO:3 and a heavy chain amino acid sequence set forth in SEQ ID NO:6, a light chain amino acid sequence set forth in SEQ ID NO:9 and a heavy chain amino acid sequence set forth in SEQ ID NO:12, a light chain amino acid sequence set forth in SEQ ID NO:15 and a heavy chain amino acid sequence set forth in SEQ ID NO:18, or a light chain amino acid sequence set forth in SEQ ID NO:21 and a heavy chain amino acid sequence set forth in SEQ ID NO:24. In a related class of embodiments, the antibody comprises a CD4 binding fragment of an antibody that comprises a light chain amino acid sequence set forth in SEQ ID NO:3 and a heavy chain amino acid sequence set forth in SEQ ID NO:6, a light chain amino acid sequence set forth in SEQ ID NO:9 and a heavy chain amino acid sequence set forth in SEQ ID NO:12, a light chain amino acid sequence set forth in SEQ ID NO:15 and a heavy chain amino acid sequence set forth in SEQ ID NO:18, or a light chain amino acid sequence set forth in SEQ ID NO:21 and a heavy chain amino acid sequence set forth in SEQ ID NO:24.

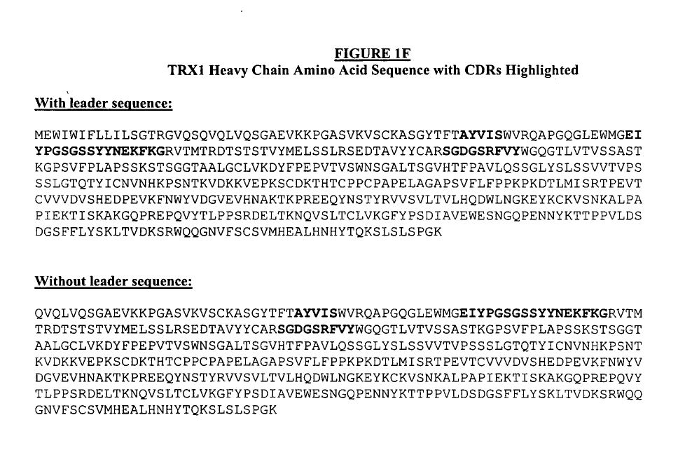

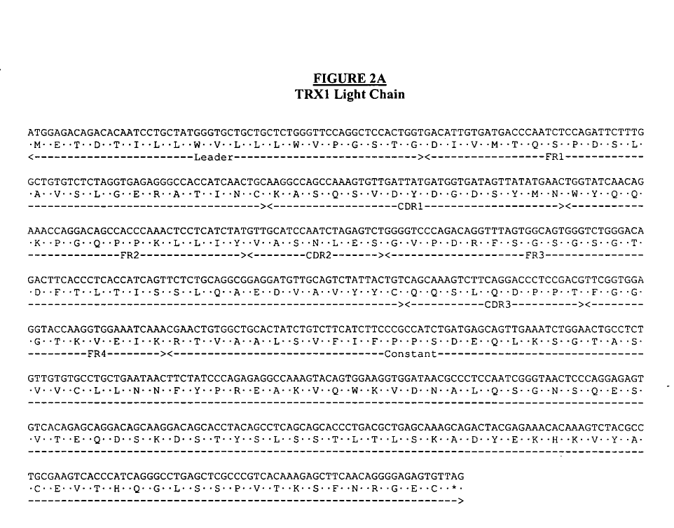

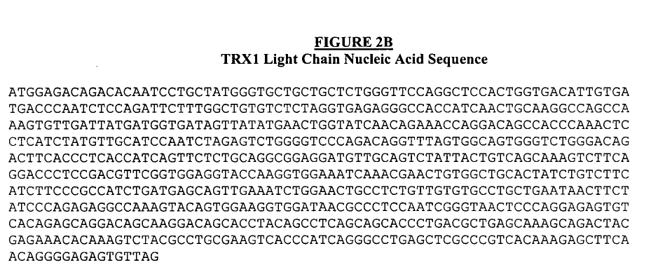

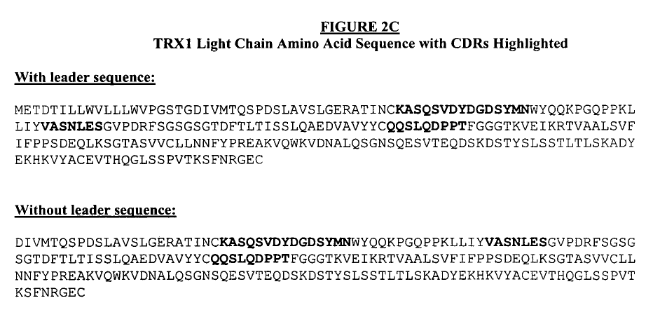

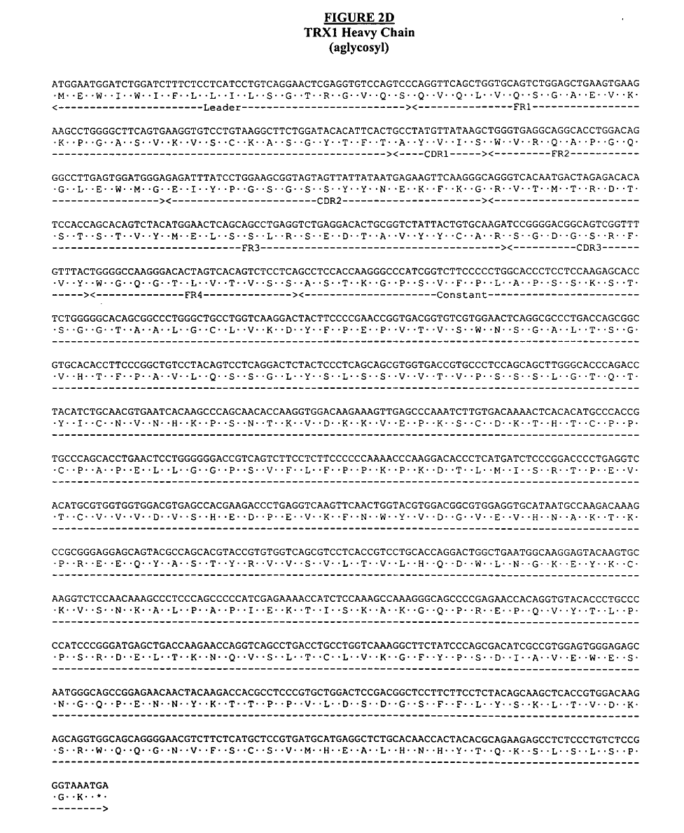

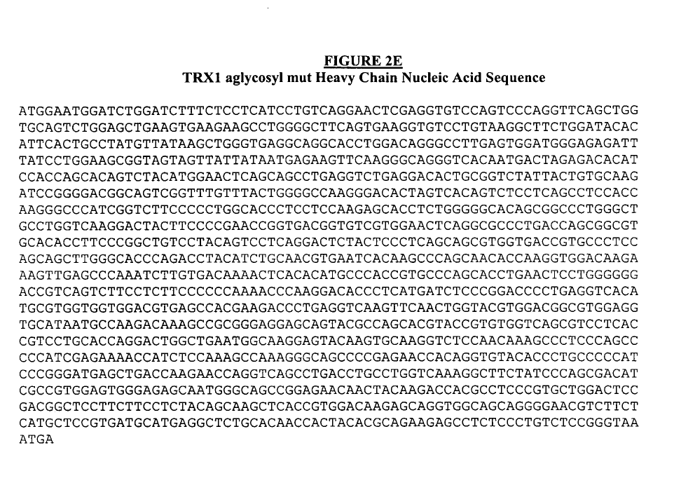

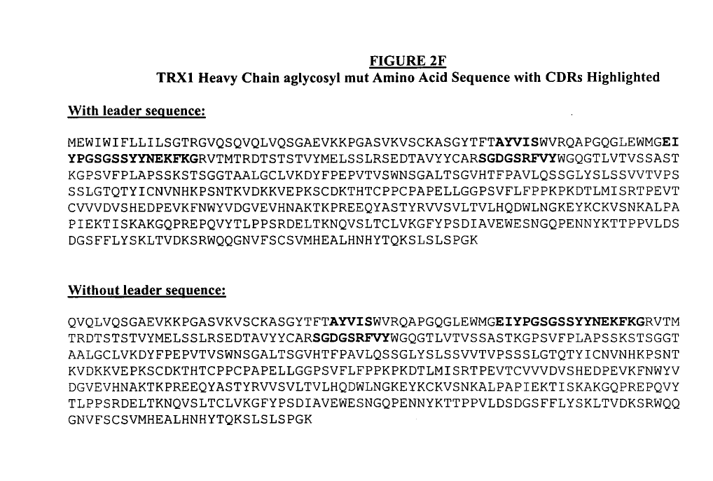

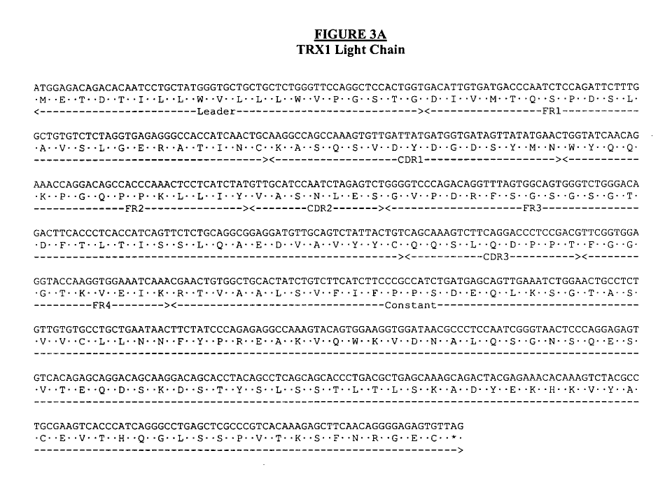

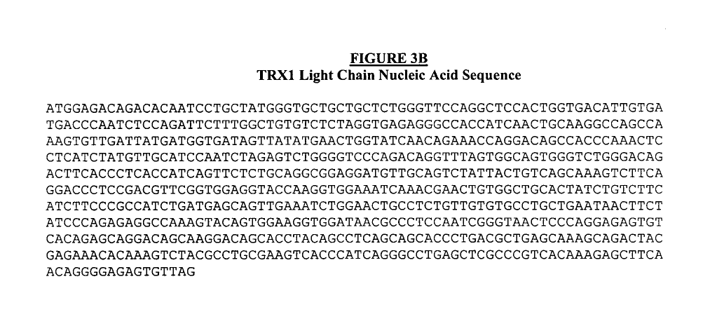

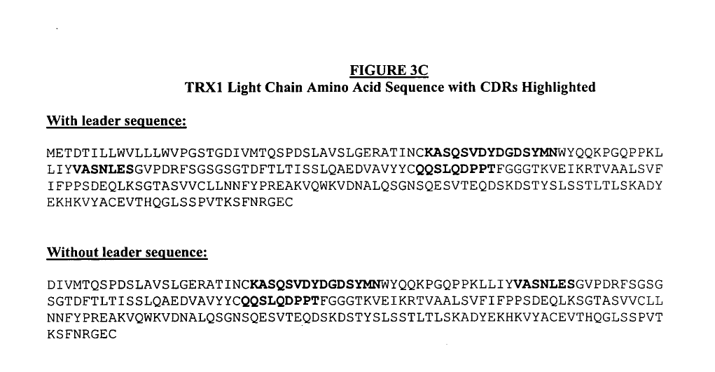

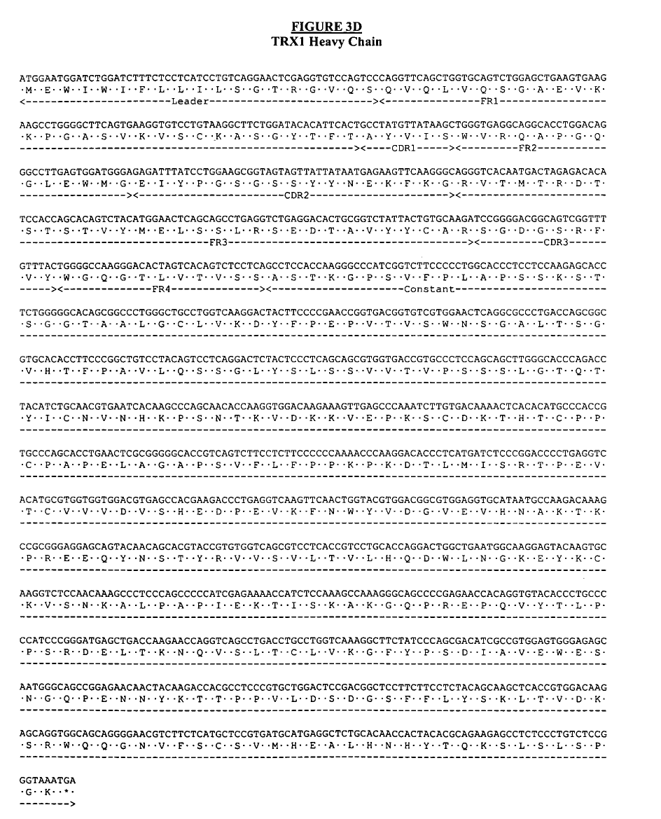

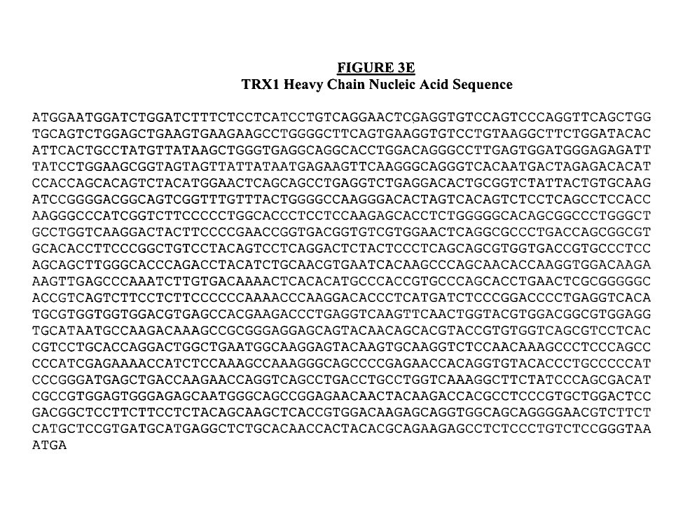

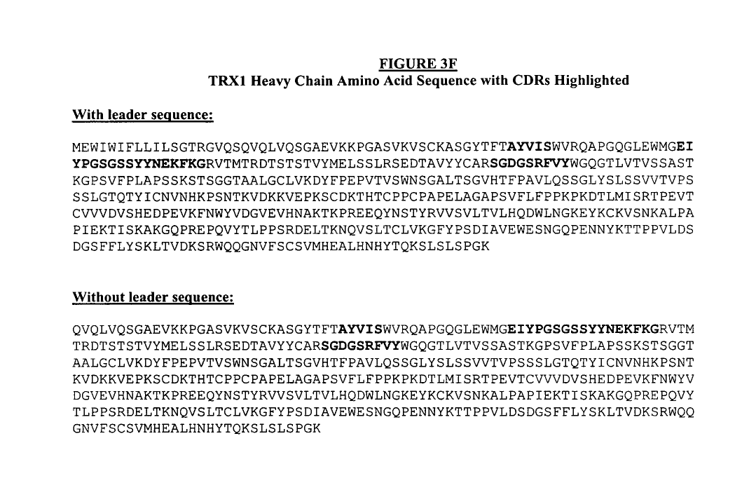

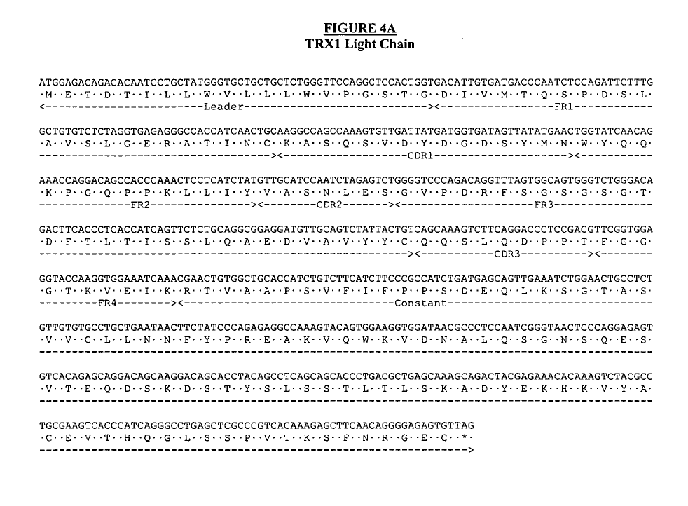

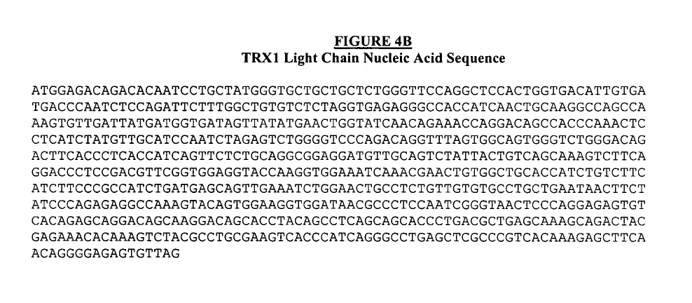

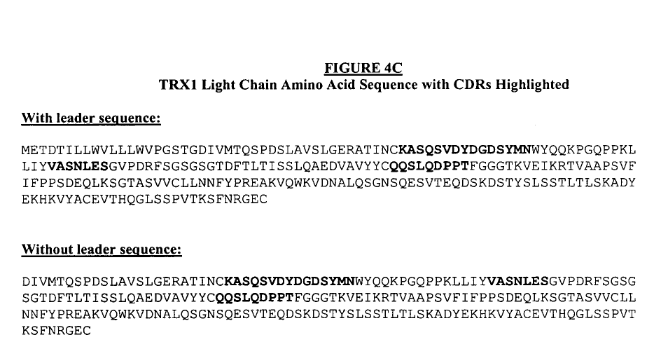

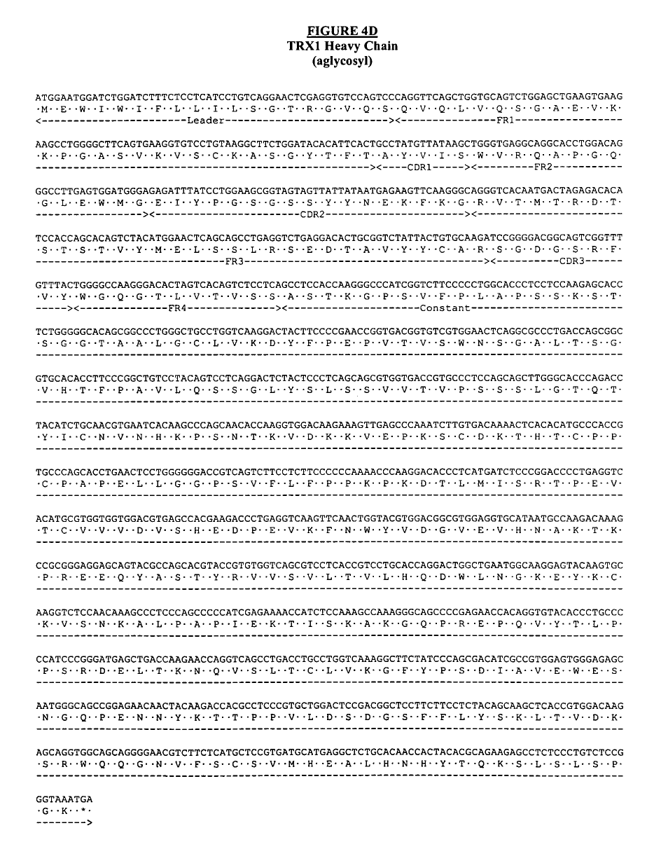

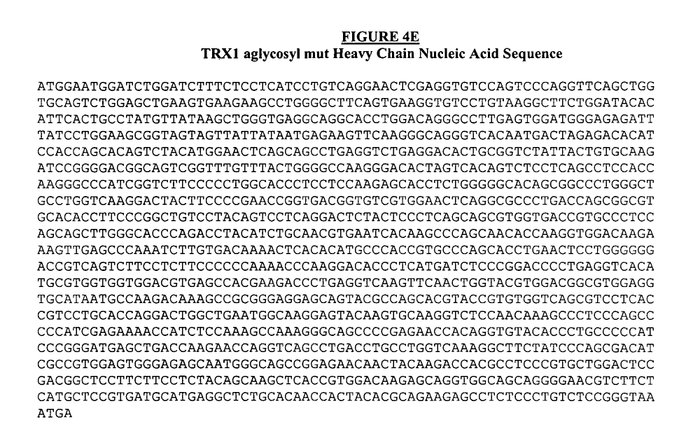

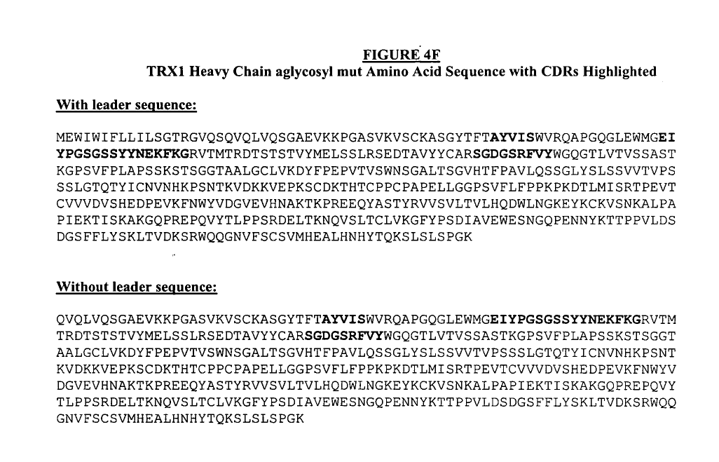

Antibodies comprising one or more CDRs from a TRX1 antibody are also useful in the methods. Thus, in one class of embodiments, the non-depleting CD4 antibody comprises CDR1 (SEQ ID NO:25), CDR2 (SEQ ID NO:26), or preferably CDR3 (SEQ ID NO:27) of the light chain shown in FIG. 1A. The antibody optionally includes CDR1, CDR2, and CDR3 of the light chain shown in FIG. 1A (SEQ ID NOs:25-27). Similarly, in one class of embodiments, the antibody comprises CDR1 (SEQ ID NO:28), CDR2 (SEQ ID NO:29), or preferably CDR3 (SEQ ID NO:30) of the heavy chain shown in FIG. 1D. The antibody optionally includes CDR1, CDR2, and CDR3 of the heavy chain shown in FIG. 1D (SEQ ID NOs:28-30). In one class of embodiments, the antibody comprises CDR1, CDR2, and CDR3 of the light chain shown in FIG. 1A and CDR1, CDR2, and CDR3 of the heavy chain shown in FIG. 1D (SEQ ID NOs:25-30). The antibody optionally also includes FR1, FR2, and/or FR3 of the light chain shown in FIG. 1A, FIG. 2A, FIG. 3A, or FIG. 4A and/or FR1, FR2, FR3, and/or FR4 of the heavy chain shown in FIG. 1D, FIG. 2D, FIG. 3D, or FIG. 4D.

Other exemplary antibodies include, but are not limited to, antibodies that bind the same epitope as a TRX1 antibody (e.g., as an antibody shown in any one of FIGS. 1-4).

Where the subject is a human, the antibody is preferably a humanized or human antibody. It will be evident that for treatment of a non-human mammal, the antibody is optionally adapted for use in that animal, for example, by incorporation of framework and constant region sequences from an immunoglobulin from a mammal of the appropriate species. The antibody is optionally a monoclonal antibody, an intact antibody, an antibody fragment, and/or a native antibody.

The antibody optionally has a reduced effector function, e.g., as compared to human IgG1, such that its ability to induce complement activation and/or antibody dependent cell-mediated cytotoxicity is decreased. For example, the antibody can have reduced (or no) binding to the Fc receptor. Similarly, the antibody can have an aglycosylated Fc portion. The antibody optionally may be an anti-CD4 variant antibody having the ability to bind FcRN.

### Treatment of Lupus

In one aspect, the invention provides methods of treating lupus in a mammalian subject, e.g., a human subject, by administering a therapeutically effective amount of an anti-CD4 non-depleting antibody and/or a second agent. The lupus for which the subject is treated is typically systemic lupus erythematosus (SLE), cutaneous lupus erythematosus (CLE), or lupus nephritis, but can be another form of lupus such as extrarenal, cerebritis, pediatric, non-renal, discoid, or alopecia. The lupus to be treated can be early, mid, or late stage disease when treatment is initiated. In embodiments in which lupus nephritis is treated, the lupus nephritis can be any one of classes I-VI. For example, the lupus to be treated can be mesangioproliferative lupus nephritis (class II) or membanous lupus nephritis (class V). Typically the lupus is proliferative lupus nephritis (class III or class IV), treated with the goal of achieving a reduction in proteinuria, a reduction in active urinary sediment, and normalization or stabilization of renal function. For example, proteinuria (measured as established in the art, for example, in a 24 hour urine protein measurement, using a dip stick or other routine analysis, e.g., as described in Example 1 herein) can be reduced by at least 25% or by at least 50%, or even by at least 75% or by at least 90%, or the proteinuria can be reduced to less than 1 g per day or less than 500 mg per day. As another example, the subject's urine protein to creatinine ratio can be reduced by at least 25% or by at least 50%, or the ratio can be reduced to less than 1 or less than 0.5. Similarly, active urinary sediment (monitored as established in the art, for example, by microscopic observation) can be reduced by at least 25%, by at least 50%, by at least 75%, or by at least 90%, or only inactive urinary sediment (as evidenced by less than 10 red blood cells/high power field and absence of red cell casts, and preferably by less than 5 red blood cells/high power field) may remain after initiation of treatment. In one aspect, when lupus nephritis is treated, the subject displays a reduction in proteinuria and/or a reduction in active urinary sediment after initiation of treatment with the combination. For example, protein concentration in the urine of the subject can be reduced to less than 75%, less than 50%, less than 25%, or less than 10% of the concentration in the urine of the subject prior to initiation of treatment with the combination, or to less than 1 g per day or less than 500 mg per day, and/or active urinary sediment can be reduced by at least 25%, by at least 50%, by at least 75%, or by at least 90%, or only inactive urinary sediment may remain after initiation of treatment (e.g., less than 10 and preferably less than 5 red blood cells/high power field).

In one general class of embodiments, in the methods a therapeutically effective amount of a combination of a non-depleting CD4 antibody and at least a second compound is administered to the subject to treat lupus. The non-depleting CD4 antibody can be any of those described herein. The second compound is typically one that is used to treat lupus, for example, a standard of care or experimental treatment. Exemplary second compounds include, but are not limited to, a cytotoxic agent; an immunosuppressive agent; an anti-malarial drug such as, e.g., hydroxychloroquine, chloroquine, or quinacrine; a chemotherapeutic agent; a cytokine antagonist or antibody; a growth factor; a hormone (e.g., hormone replacement treatment); anti-hormonal therapy; an integrin antagonist or antibody, e.g., an α4-integrin antibody or antagonist; a B-cell surface marker antagonist; an antibody to a B-cell surface marker (e.g., a CD20 antibody, e.g., Rituximab, also known as Rituxan®); a CD5, CD28, or CD40 antibody or blocking agent; a corticosteroid, e.g., methylprednisolone, prednisone such as low-dose prednisone, dexamethasone, or glucorticoid, e.g., via joint injection, including systemic corticosteroid therapy; a DMARD; or a combination of any of the above, etc. See also U.S. patent application publications 2006/0024295 and 2003/0219403.

In one class of embodiments, the second compound is selected from, e.g., cyclophosphamide, mycophenolate mofetil, CTLA4-Ig, and BR3-Fc. Cyclophosphamide is also known by the brand name Cytoxan®. Mycophenolate mofetil is also called CellCept®, MMF, or 2-morpholinoethyl (E)-6-(1,3-dihydro-4-hydroxy-6-methoxy-7-methyl-3-oxo-5-isobenzofuranyl)-4-methyl-4-hexenoate. CTLA4-Ig is an extracellular domain of human cytotoxic T-lymphocyte-associated antigen 4 (CTLA-4) linked to a modified Fc portion of a human immunoglobulin, for example, abatacept (Orencia® from Bristol-Myers Squibb) or RG2077 from RepliGen. An exemplary α4-integrin antibody is natalizumab (Tysabri®). BR3-Fc, a soluble antagonist of BAFF, is a fusion protein that includes the extracellular domain of human BR3 (a BAFF receptor found on B cells) and human IgG1 Fc (see, e.g., Vugmeyster et al. (2006) American Journal of Pathology 168:476-489 and Kayagaki et al. (2002) Immunity 10:515-524). A third, fourth, etc. compound is optionally included in the combination; as just one example, a corticosteroid such as methylprednisolone and/or prednisone can be administered along with the CD4 antibody and cyclophosphamide.

In one embodiment, the subject has never been previously treated with drug(s), such as immunosuppressive agent(s), to treat the lupus and/or has never been previously treated with an anti-CD4 antibody. In another embodiment, the subject has been previously treated with drug(s) to treat the lupus and/or has been previously treated with an anti-CD4 antibody. In a further embodiment, the subject does not have rheumatoid arthritis. In a still further embodiment, the subject does not have multiple sclerosis. In yet another embodiment, the subject does not have an autoimmune disease other than lupus. An “autoimmune disease” herein is a disease or disorder arising from and directed against an individual's own tissues or organs or a co-segregate or manifestation thereof or resulting condition therefrom. In one embodiment, it refers to a condition that results from, or is aggravated by, the production by B cells of antibodies that are reactive with normal body tissues and antigens. In other embodiments, the autoimmune disease is one that involves secretion of an autoantibody that is specific for an epitope from a self antigen (e.g. a nuclear antigen).

In one embodiment, prior to initiation of treatment with the combination, the subject displays proteinuria, which proteinuria is ameliorated by the treatment. For example, prior to initiation of treatment, the subject can display proteinuria greater than 500 mg per day, greater than 1000 mg per day, greater than 2000 mg per day, or greater than 3500 mg per day; after initiation of treatment, the proteinuria can be reduced by at least 25% or by at least 50%, or even by at least 75% or by at least 90%, or the proteinuria can be reduced to less than 1 g per day or less than 500 mg per day, for example, as determined by a 24 hour urine protein measurement.

A decrease in protein to creatinine ratio can be similarly monitored. Urine protein and creatinine levels can be measured as established in the art, for example, by determination of spot urine protein to creatinine ratio, e.g., of a random urine sample. In one embodiment, prior to initiation of treatment with the combination, the subject displays a protein to creatinine ratio of greater than 0.5, greater than 1, or greater than 2; after initiation of treatment, the protein to creatinine ratio can be reduced, e.g., to less than 1 (e.g., for a partial response to treatment) or to less than 0.5 (e.g., for a full response). After initiation of treatment, the protein to creatinine ratio can be reduced by at least 25% or by at least 50% compared to the pre-treatment value. In one embodiment, prior to initiation of treatment with the combination, the subject displays nephrotic range proteinuria, with a protein to creatinine ratio of greater than 3; after initiation of treatment, the protein to creatinine ratio is reduced to less than 3, or optionally by at least 25% or by at least 50% or to less than 2 or less than 1.

Response to treatment of lupus, including lupus nephritis, with the combination can also be assessed, for example, by monitoring complement levels, autoantibody levels, and/or overall disease activity. For example, normalization of complement levels (e.g., C3, C4, and CH50) can be indicative of successful treatment. Similarly, after initiation of treatment, levels of autoantibodies such as anti-double-stranded DNA antibodies, ANA, and anti-C1q can be reduced, e.g., by at least 25%, by at least 50%, or by at least 75%. Improvement in renal biopsy can also be observed as indicative of successful treatment.

Optionally, prior to initiation of treatment with the combination, the subject displays nephrotic syndrome. Diagnosis of nephrotic syndrome can be performed as established in the art. Some signs, symptoms, or other indicators that can be used to diagnose nephrotic syndrome include 24 hour urine protein greater than 3.5 g/day, protein to creatinine ratio greater than 3, hypoalbuminemia (low level of albumin in the blood), edema (swelling), especially around the eyes, feet, and hands, and/or hypercholesterolemia (high level of cholesterol in the blood). The invention is not limited to these signs, symptoms, or other indicators of nephrotic syndrome. The nephrotic syndrome is optionally ameliorated by treatment with the combination. For example, the subject optionally displays a reduction in proteinuria to less than 3.5 g/day after initiation of the treatment, e.g., to less than 3 g/day, less than 2 g/day, less than 1 g/day, or even less than 0.5 g/day, and/or a reduction in protein to creatinine ratio to less than 3 after initiation of the treatment, e.g., to less than 2, less than 1, or even less than 0.5.

Treatment of the subject with the combination can have considerable benefits for the subject, for example, in reduction in undesirable side effects. For example, the amount of second compound (e.g., cyclophosphamide) required for treatment in combination with the non-depleting CD4 antibody can be considerably less than the amount required to ameliorate symptoms through treatment with the second compound alone. For example, cyclophosphamide can produce serious side effects; use of less of the drug to achieve treatment, therefore, is highly desirable.

In one aspect, the methods include treating the subject with the non-depleting CD4 antibody and the second compound to reduce symptoms, and then continuing treatment of the subject with the non-depleting CD4 antibody (not in combination with the second compound) to maintain remission. For example, the subject can be treated with a combination of the non-depleting CD4 antibody and cyclophosphamide, mycophenolate mofetil, or CTLA4-Ig to reduce symptoms, and then treated with the non-depleting CD4 antibody alone (i.e., not in combination with the cyclophosphamide, mycophenolate mofetil, or CTLA4-Ig) to maintain remission. Such methods can also reduce side effects, by minimizing exposure of the subject to the second compound. In another embodiment, the subject is treated with the non-depleting CD4 antibody and the second compound to reduce symptoms, and then treatment of the subject with the second compound or one or more other compounds, but other than the non-depleting CD4 antibody, is continued to maintain remission.

Another general class of embodiments also provides methods of treating lupus nephritis in a mammalian subject, e.g., a human. In the methods, a therapeutically effective amount of a non-depleting CD4 antibody is administered to the subject. After initiation of treatment with the antibody, the subject displays an improvement in renal function, a reduction in proteinuria, and/or a reduction in active urinary sediment, as compared to the level(s) of proteinuria and/or active urinary sediment displayed by the subject prior to initiation of treatment. For example, proteinuria can be reduced by at least 25%, by at least 50%, by at least 75%, or by at least 90%, or the proteinuria can be reduced to less than 1 g per day or less than 500 mg per day; urine protein to creatinine ratio can be reduced by at least 25% or by at least 50%, or the ratio can be reduced to less than 1 or less than 0.5; and/or active urinary sediment can be reduced by at least 25%, by at least 50%, by at least 75%, or by at least 90%, or only inactive urinary sediment may remain after initiation of treatment. The non-depleting CD4 antibody can be any of those described herein.

In one embodiment, the subject has never been previously treated with drug(s) to treat the lupus nephritis and/or has never been previously treated with an anti-CD4 antibody. In another embodiment, the subject has been previously treated with drug(s) to treat the lupus nephritis and/or has been previously treated with an anti-CD4 antibody. In another embodiment, the non-depleting anti-CD4 antibody of the invention is the only medicament administered to the subject to treat the lupus nephritis. In another embodiment, the non-depleting CD4 antibody of the invention is one of the medicaments used to treat the lupus nephritis. In a further embodiment, the subject does not have rheumatoid arthritis. In a still further embodiment, the subject does not have multiple sclerosis. In yet another embodiment, the subject does not have an autoimmune disease other than lupus and/or lupus nephritis.

In one class of embodiments, the methods include administration of at least a second compound such as any of those described herein in combination with the non-depleting CD4 antibody. For example, cyclophosphamide, mycophenolate mofetil, CTLA4-Ig, or an α4-integrin antibody may be administered to the subject in combination with the non-depleting CD4 antibody. A third, fourth, etc. compound is optionally included in the combination; for example, a corticosteroid such as methylprednisolone and/or prednisone can be administered along with the non-depleting CD4 antibody and cyclophosphamide.

In one embodiment, prior to initiation of treatment with the non-depleting CD4 antibody, the subject displays proteinuria, which proteinuria is reduced after initiation of treatment with the non-depleting CD4 antibody. For example, prior to initiation of treatment, the subject can display proteinuria greater than 500 mg per day, greater than 1000 mg per day, greater than 2000 mg per day, or greater than 3500 mg per day; after initiation of treatment, the proteinuria can be reduced by at least 25%, by at least 50%, by at least 75%, or by at least 90%, or the proteinuria can be reduced to less than 1 g per day or less than 500 mg per day. A decrease in protein to creatinine ratio can be similarly monitored. In one embodiment, prior to initiation of treatment, the subject displays a protein to creatinine ratio of greater than 0.5, greater than 1, or greater than 2; after initiation of treatment, the protein to creatinine ratio can be reduced by at least 25% or by at least 50%, or to less than 1 or to less than 0.5. In one embodiment, prior to initiation of treatment with the combination, the subject displays nephrotic range proteinuria, with a protein to creatinine ratio of greater than 3; after initiation of treatment, the protein to creatinine ratio is reduced to less than 3, or optionally by at least 25% or by at least 50% or to less than 2 or less than 1. Optionally, prior to initiation of treatment, the subject displays nephrotic syndrome. The nephrotic syndrome is optionally ameliorated by treatment. For example, the subject optionally displays a reduction in proteinuria to less than 3.5 g/day after initiation of the treatment, e.g., to less than 3 g/day, less than 2 g/day, less than 1 g/day, or even less than 1 g/day or less than 0.5 g/day.

### Treatment of Multiple Sclerosis

Multiple Sclerosis (MS) is an inflammatory and demyelinating degenerative disease of the human central nervous system (CNS). It is a worldwide disease that affects approximately 300,000 persons in the United States; it is a disease of young adults, with 70%-80% having onset between 20 and 40 years old (Anderson et al. Ann Neurology 31(3): 333-6 (1992); Noonan et al. Neurology 58: 136-8 (2002)). MS is a heterogeneous disorder based on clinical course, magnetic resonance imaging (MRI) scan assessment, and pathology analysis of biopsy and autopsy material (Lucchinetti et al. Ann Neurol 47: 707-17 (2000)). The disease manifests itself in a large number of possible combinations of deficits, including spinal cord, brainstem, cranial nerve, cerebellar, cerebral, and cognitive syndromes. Progressive disability is the fate of most patients with MS, especially when a 25-year perspective is included. Half of MS patients require a cane to walk within 15 years of disease onset. MS is a major cause of neurologic disability in young and middle-aged adults and, until the past decade, has had no known beneficial treatments. MS is difficult to diagnose because of the non-specific clinical findings, which led to the development of highly structured diagnostic criteria that include several technological advances, consisting of MRI scans, evoked potentials, and cerebrospinal fluid (CSF) studies. All diagnostic criteria rely upon the general principles of scattered lesions in the central white matter occurring at different times and not explained by other etiologies such as infection, vascular disorder, or another autoimmune disorder (McDonald et al. Ann Neurol 50: 121-7 (2001)). MS has four patterns of disease: relapsing-remitting MS (RRMS; 80%-85% of cases at onset), primary progressive MS (PPMS; 10%-15% at onset), progressive relapsing MS (PRMS; 5% at onset); and secondary progressive MS (SPMS) (Kremenchutzky et al. Brain 122 (Pt 10): 1941-50 (1999); Confavreux et al. N Engl J Med 343(20): 1430-8 (2000)). An estimated 50% of patients with RRMS will develop SPMS in 10 years, and up to 90% of RRMS patients will eventually develop SPMS (Weinshenker et al. Brain 112 (Pt 1): 133-46 (1989)).

The invention includes methods of treating multiple sclerosis in a mammalian subject, e.g., a human subject. In one aspect, the methods include administering to the subject a therapeutically effective amount of a non-depleting CD4 antibody. The non-depleting CD4 antibody can be any of these described herein. In another aspect, the methods include administering to the subject a therapeutically effective amount of a combination of a non-depleting CD4 antibody and at least a second compound. Again, the non-depleting CD4 antibody can be any of these described herein.

The second compound is typically one that is used to treat MS, for example, a standard of care or experimental treatment. Exemplary second compounds include, but are not limited to, a cytotoxic agent; an immunosuppressive agent (e.g., cyclophosphamide); a B-cell surface marker antagonist; an antibody to a B-cell surface marker; a CD20 antibody, e.g., Rituximab, see US 20060051345); a CD5, CD28, or CD40 antibody or blocking agent; a corticosteroid (e.g., prednisone), CTLA4-Ig, an α4-integrin antibody or antagonist such as natalizumab (Tysabri®), mycophenolate mofetil, a statin, an LFA-1 or CD-11a antibody or blocking agent (see U.S. patent application publication 20050281817 by Jardieu et al. entitled “Method for treating multiple sclerosis”), an interleukin-12 antibody, a beta interferon (e.g., an interferon β-1a such as Avonex® or Rebif®, or an interferon β-1b such as Betaseron®), glatiramer acetate (Copaxone®), a CD52 antibody such as alemtuzuman (CamPath®), an interleukin receptor antibody such as daclizumab (Zenapax®, an antibody to the interleukin-2 receptor alpha subunit), etc.

In one class of embodiments, the methods include treating the subject with the non-depleting CD4 antibody and the second compound to reduce symptoms, and then continuing treatment of the subject with the non-depleting CD4 antibody (not in combination with the second compound) to maintain remission. For example, the subject can be treated with a combination of the non-depleting CD4 antibody and glatiramer acetate, and then treated with the non-depleting CD4 antibody alone to maintain remission. In another embodiment, the subject is treated with the non-depleting CD4 antibody and the second compound to reduce symptoms, and then treatment is continued with the second compound, or one or more compounds typically used to treat MS, other than the non-depleting CD4 antibody.

In one embodiment, the subject has never been previously treated with drug(s), such as immunosuppressive agent(s), to treat the multiple sclerosis and/or has never been previously treated with an anti-CD4 antibody. In another embodiment, the subject has been previously treated with drug(s) to treat the multiple sclerosis and/or has been previously treated with an anti-CD4 antibody.

Typically, the subject is eligible for treatment for multiple sclerosis, i.e., the subject is an MS subject. For the purposes herein, such MS subject is one who is experiencing, has experienced, or is likely to experience, one or more signs, symptoms or other indicators of multiple sclerosis; has been diagnosed with multiple sclerosis, whether, for example, newly diagnosed (with “new onset” MS), previously diagnosed with a new relapse or exacerbation, previously diagnosed and in remission, etc; and/or is at risk for developing multiple sclerosis. One suffering from or at risk for suffering from multiple sclerosis may optionally be identified as one who has been screened for elevated levels of CD20-positive B cells in serum, cerebrospinal fluid (CSF) and/or MS lesion(s) and/or is screened for using an assay to detect autoantibodies, assessed qualitatively, and preferably quantitatively. Exemplary such autoantibodies associated with multiple sclerosis include anti-myelin basic protein (MBP), anti-myelin oligodendrocytic glycoprotein (MOG), anti-ganglioside and/or anti-neurofilament antibodies. Such autoantibodies may be detected in the subject's serum, cerebrospinal fluid (CSF) and/or MS lesion. By “elevated” autoantibody or B cell level(s) herein is meant level(s) of such autoantibodies or B cells which significantly exceed the level(s) in an individual without MS.

The MS to be treated herein includes primary progressive multiple sclerosis (PPMS), relapsing-remitting multiple sclerosis (RRMS), secondary progressive multiple sclerosis (SPMS), and progressive relapsing multiple sclerosis (PRMS). The MS can be early, mid, or late stage disease when treatment is initiated. The expression “therapeutically effective amount” with reference to treatment of MS refers to an amount of the antibody (or combination of the antibody and at least the second compound) that is effective for preventing, ameliorating or treating the multiple sclerosis. Such an effective amount will generally result in an improvement in the signs, symptoms or other indicators of MS, such as reducing relapse rate, preventing disability, reducing number and/or volume of brain MRI lesions, improving timed 25-foot walk, extending the time to disease progression (e.g. using Expanded Disability Status Scale, EDSS), etc. In one aspect, demyelination is decreased in the treated subject.

“Primary progressive multiple sclerosis” or “PPMS” is characterized by a gradual progression of the disease from its onset with no superimposed relapses and remissions at all. There may be periods of a leveling off of disease activity and there may be good and bad days or weeks. PPMS differs from RRMS and SPMS in that onset is typically in the late thirties or early forties, men are as likely women to develop it, and initial disease activity is often in the spinal cord and not in the brain. PPMS often migrates into the brain, but is less likely to damage brain areas than RRMS or SPMS; for example, people with PPMS are less likely to develop cognitive problems. PPMS is the sub-type of MS that is least likely to show inflammatory (gadolinium enhancing) lesions on MRI scans. The Primary Progressive form of the disease affects between 10 and 15% of all people with multiple sclerosis. PPMS may be defined according to the criteria in McDonald et al. Ann Neurol 50: 121-7 (2001). The subject with PPMS treated herein is usually one with probable or definitive diagnosis of PPMS.

“Relapsing-remitting multiple sclerosis” or “RRMS” is characterized by relapses (also known as exacerbations) during which time new symptoms can appear and old ones resurface or worsen. The relapses are followed by periods of remission, during which time the person fully or partially recovers from the deficits acquired during the relapse. Relapses can last for days, weeks or months and recovery can be slow and gradual or almost instantaneous. The vast majority of people presenting with MS are first diagnosed with RRMS. This is typically when they are in their twenties or thirties, though diagnoses much earlier or later are known. Twice as many women as men present with this sub-type of MS. During relapses, myelin, a protective insulating sheath around the nerve fibers (neurons) in the white matter regions of the central nervous system (CNS), may be damaged in an inflammatory response by the body's own immune system. This causes a wide variety of neurological symptoms that vary considerably depending on which areas of the CNS are damaged. Immediately after a relapse, the inflammatory response dies down and a special type of glial cell in the CNS (called an oligodendrocyte) sponsors remyelination—a process whereby the myelin sheath around the axon may be repaired. It is this remyelination that may be responsible for the remission. Approximately 50% of patients with RRMS convert to SPMS within 10 years of disease onset. After 30 years, this figure rises to 90%. At any one time, the relapsing-remitting form of the disease accounts around 55% of all people with MS.

“Secondary progressive multiple sclerosis” or “SPMS” is characterized by a steady progression of clinical neurological damage with or without superimposed relapses and minor remissions and plateaux. People who develop SPMS will have previously experienced a period of RRMS which may have lasted anything from two to forty years or more. Any superimposed relapses and remissions there are, tend to tail off over time. From the onset of the secondary progressive phase of the disease, disability starts advancing much quicker than it did during RRMS though the progress can still be quite slow in some individuals. After 10 years, 50% of people with RRMS will have developed SPMS. By 25 to 30 years, that figure will have risen to 90%. SPMS tends to be associated with lower levels of inflammatory lesion formation than in RRMS but the total burden of disease continues to progress. At any one time, SPMS accounts around 30% of all people with multiple sclerosis.

“Progressive relapsing multiple sclerosis” refers to “PRMS” is characterized by a steady progression of clinical neurological damage with superimposed relapses and remissions. There is significant recovery immediately following a relapse but between relapses there is a gradual worsening of symptoms. PRMS affects around 5% of all people with multiple sclerosis. Some neurologists believe PRMS is a variant of PPMS.

### Treatment of Other Conditions

Non-depleting CD4 antibodies, including combinations of non-depleting CD4 antibodies and one or more other compounds, are also useful for treating disorders and conditions other than lupus or multiple sclerosis, for example, pathological conditions to which CD4⁺ T cells contribute. Thus, one aspect of the invention provides methods of treating a condition in a mammalian subject, e.g., a human subject. The methods include administering to the subject a therapeutically effective amount of a combination of a non-depleting CD4 antibody and at least a second compound. In one embodiment, the subject is a tissue transplant recipient, and the condition to be treated is transplant rejection or graft versus host disease. Other conditions that can be treated with the combination include, but are not limited to, autoimmune disorders or diseases such as rheumatoid arthritis, asthma, psoriasis, inflammatory bowel disease (e.g., Crohn's disease or ulcerative colitis), and Sjogren's syndrome.

The non-depleting CD4 antibody can be any of these described herein. The second compound is optionally one that is used to treat the condition, for example, a standard of care or experimental treatment. Exemplary second compounds include, but are not limited to, a cytotoxic agent; an immunosuppressive agent (e.g., cyclophosphamide); a B-cell surface marker antagonist; an antibody to a B-cell surface marker; a CD20 antibody, e.g., Rituximab, see US 20060051345); a CD5, CD28, or CD40 antibody or blocking agent; a corticosteroid (e.g., prednisone), CTLA4-Ig, an α4-integrin antibody or antagonist such as natalizumab (Tysabri®), mycophenolate mofetil, a statin, an LFA-1 or CD-11a antibody or blocking agent (see U.S. patent application publication 20050281817 by Jardieu et al. entitled “Method for treating multiple sclerosis”), an interleukin-12 antibody, a beta interferon (e.g., an interferon β-1a such as Avonex® or Rebif®, or an interferon β-1b such as Betaseron®), glatiramer acetate (Copaxone®), a CD52 antibody such as alemtuzuman (CamPath®), an interleukin receptor antibody such as daclizumab (Zenapax®, an antibody to the interleukin-2 receptor alpha subunit), etc. Additional exemplary second compounds are described herein and/or known in the art. Optionally, the second compound is selected from the group consisting of cyclophosphamide, mycophenolate mofetil, and CTLA4-Ig.

In one class of embodiments, the methods include treating the subject with the non-depleting CD4 antibody and the second compound to reduce symptoms, and then continuing treatment of the subject with the non-depleting CD4 antibody (not in combination with the second compound) to maintain remission. In another embodiment, the subject is treated with the non-depleting CD4 antibody and the second compound to reduce symptoms, and then treatment is continued with the second compound, or one or more compounds typically used to treat the condition.

In one embodiment, the subject has never been previously treated with drug(s), such as immunosuppressive agent(s), to treat the condition and/or has never been previously treated with an anti-CD4 antibody. In another embodiment, the subject has been previously treated with drug(s) to treat the condition and/or has been previously treated with an anti-CD4 antibody.

Typically, the subject is eligible for treatment for the condition. For the purposes herein, such subject is one who is experiencing, has experienced, or is likely to experience, one or more signs, symptoms or other indicators of the condition; has been diagnosed with the condition, whether, for example, newly diagnosed, previously diagnosed with a new relapse or exacerbation, previously diagnosed and in remission, etc; and/or is at risk for developing the condition. For example, a subject eligible for treatment of transplant rejection or graft versus host disease can be anticipating a tissue transplant or can have already received such transplant, and in the latter case can be one who is experiencing, has experienced, or is likely to experience one or more signs, symptoms or other indicators of transplant rejection or graft versus host disease. Symptoms and indicators to such conditions, and of various autoimmune diseases and disorders, are well known in the art.

### Antibody Production and Administration

The methods of the present invention use an antibody that binds CD4. In one aspect, the anti-CD4 antibodies are non-depleting antibodies. Accordingly, methods for generating such antibodies will be described here.

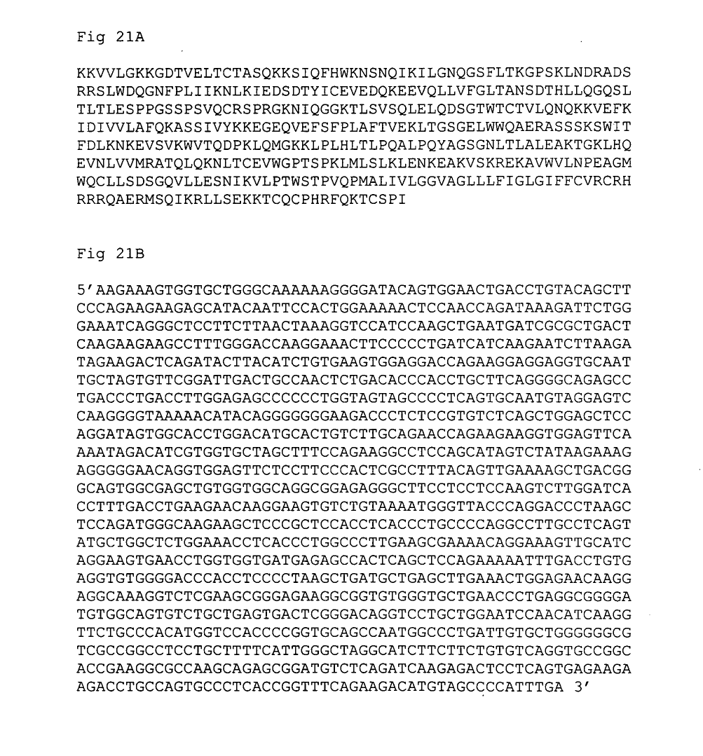

CD4 antigen to be used for production of, or screening for, antibody(ies) may be, e.g., a soluble form of CD4, such as human CD4, or a portion thereof, containing the desired epitope. The nucleic acid and amino acid sequences of human CD4 are shown in FIG. 21. Alternatively, or additionally, cells expressing CD4 at their cell surface can be used to generate, or screen for, antibody(ies). Other forms of CD4 useful for generating antibodies will be apparent to those skilled in the art.

A description follows as to exemplary techniques for the production of the antibodies used in accordance with the present invention. For additional information, see U.S. patent application publication 2003/0108518 by Frewin et al. entitled “TRX1 antibody and uses therefor” and U.S. patent application publication 2003/0219403 by Frewin et al. entitled “Compositions and methods of tolerizing a primate to an antigen,” both of which are incorporated herein by reference in their entirety for all purposes, including with respect to procedures for producing non-depleting CD4 antibodies, such as the TRX1 antibody.

Polyclonal Antibodies

Polyclonal antibodies are preferably raised in animals by multiple subcutaneous or intraperitoneal injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R′N═C═NR, where R and R′ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal Antibodies

Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Myeloma cells useful for preparation of hybridomas are those that fuse efficiently, support stable high-level production of antibody by the selected antibody-producing cells, and are sensitive to a medium such as HAT medium. Among these, a non-limiting list of myeloma cell lines includes murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. The binding specificity of monoclonal antibodies produced by hybridoma cells may be determined by immunoprecipitation or by an in vitro binding assay, such as radioimmunoassay (RIA) or enzyme-linked immunoabsorbent assay (ELISA). The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose® crosslinked agarose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a useful source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high-affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin-coding sequence all or part of the coding sequence for a non-immunoglobulin polypeptide.

Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Humanized Antibodies

Methods for humanizing non-human antibodies have been described in the art. Preferably, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable-region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable-region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light- or heavy-chain variable regions. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain-joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage-display technology (McCafferty et al., Nature 348:552-553 (1990)) can be used to produce human antibodies and antibody fragments in vitro, from immunoglobulin variable (V)-domain gene repertoires from unimmunized donors. According to this technique, antibody V-domain genes are cloned in frame into either a major or minor coat-protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

Human antibodies may also be generated by in vitro-activated B cells (see U.S. Pat. Nos. 5,567,610 and 5,229,275).

Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host-cell culture. Other techniques for the production of antibody fragments will be apparent to the skilled practitioner. In other embodiments, the antibody of choice is a single-chain Fv fragment (scFv). See WO 1993/16185 and U.S. Pat. Nos. 5,571,894 and 5,587,458. The antibody fragment may also be a “linear antibody”, e.g., as described in U.S. Pat. No. 5,641,870. Such linear antibody fragments may be monospecific or bispecific.

Bispecific Antibodies

Bispecific antibodies are antibodies that have binding specificities for at least two different epitopes. Exemplary bispecific antibodies may bind to two different epitopes of the CD4 antigen. Other such antibodies may bind CD4 and further bind a second T-cell surface marker. Bispecific antibodies may also be used to localize drugs or cytotoxic agents to the T cell; these antibodies possess a CD4-binding arm and an arm that binds the drug or cytotoxic agent. Bispecific antibodies can be prepared as full-length antibodies or antibody fragments (e.g. F(ab′)₂ bispecific antibodies).

Methods for making bispecific antibodies are known in the art. Traditional production of full-length bispecific antibodies is based on the coexpression of two immunoglobulin heavy-chain-light-chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 1993/08829, and in Traunecker et al., EMBO J., 10:3655-3659 (1991).

According to a different approach, antibody variable domains with the desired binding specificities (antibody-antigen combining sites) are fused to immunoglobulin constant-domain sequences. The fusion preferably is with an immunoglobulin heavy-chain constant domain, comprising at least part of the hinge, CH2, and CH3 regions. In one approach, the first heavy-chain constant region (CH1), containing the site necessary for light-chain binding, is present in at least one of the fusions. DNAs encoding the immunoglobulin heavy-chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy-chain-light-chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 1994/04690. For further details of generating bispecific antibodies, see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. One such interface comprises at least a part of the C_{H3} domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 1991/00360, WO 1992/200373, and EP 03089). Heteroconjugate antibodies may be made using any convenient cross-linking methods. Suitable cross-linking agents are well known in the art, and are disclosed, for example, in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

Conjugates and Other Modifications of the Antibody

The antibody used in the methods or included in the articles of manufacture herein is optionally conjugated to a drug, e.g., as described in WO 2004/032828 and U.S. patent application publication 2006/0024295. The antibodies of the present invention may also be conjugated with a prodrug-activating enzyme that converts a prodrug (e.g. a peptidyl chemotherapeutic agent, see WO 1981/01145) to an active anti-cancer drug. See, for example, WO 1988/07378, U.S. Pat. No. 4,975,278, and U.S. patent application publication 2006/0024295.

Other modifications of the antibody are contemplated herein. For example, the antibody may be linked to one of a variety of nonproteinaceous polymers, e.g., polyethylene glycol (PEG), polypropylene glycol, polyoxyalkylenes, or copolymers of polyethylene glycol and polypropylene glycol.

The antibodies disclosed herein may also be formulated as liposomes. Liposomes containing the antibody are prepared by methods known in the art, such as described in Epstein et al., Proc. Natl. Acad. Sci. USA, 82:3688 (1985); Hwang et al., Proc. Natl. Acad. Sci. USA, 77:4030 (1980); U.S. Pat. Nos. 4,485,045 and 4,544,545; and WO 1997/38731 published Oct. 23, 1997. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al. J. Biol. Chem. 257: 286-288 (1982) via a disulfide-interchange reaction. A chemotherapeutic agent is optionally contained within the liposome. See Gabizon et al. J. National Cancer Inst. 81(19)1484 (1989).

Amino acid sequence modification(s) of protein or peptide antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are useful locations for mutagenesis is called “alanine-scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody of an enzyme, or a polypeptide that increases the serum half-life of the antibody.

Another type of variant is an amino acid substitution variant. These variants have at least one amino acid residue in the antibody molecule replaced by a different residue. The sites of greatest interest for substitutional mutagenesis of antibodies include the hypervariable regions, but FR alterations are also contemplated. Conservative substitutions are shown in Table 1 under the heading of “conservative substitutions”. If such substitutions result in a change in biological activity, then more substantial changes, denominated “exemplary substitutions” in Table 1, or as further described below in reference to amino acid classes, may be introduced and the products screened.

TABLE 1
Amino acid substitutions

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; | Tyr |
| | Tyr | |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class, while conservative substitutions entail exchanging a member of one of these classes for one within the same class. Non-depleting CD4 antibodies bearing non-conservative or conservative substitutions, deletions, or additions which alter, add or delete a single amino acid or a small percentage of amino acids (typically less than 5%, more typically less than 4%, 2% or 1%) of the amino acid residues of any of the CD4 antibodies described herein are also suitable for use in the methods of the invention.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine-scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. Such altering includes deleting one or more carbohydrate moieties found in the antibody, and/or adding one or more glycosylation sites that are not present in the antibody.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered or removed. For example, in one glycosylation variant herein, one or more amino acid substitutions are introduced in an Fc region of an antibody to eliminate one or more glycosylation sites. Such an aglycosylated antibody can have reduced effector function, e.g., as compared to human IgG1, such that its ability to induce complement activation and/or antibody dependent cell-mediated cytotoxicity is decreased, and the aglycosylated antibody can have reduced (or no) binding to the Fc receptor.

For certain antibodies, e.g., a depleting antibody used as a second compound in the methods of the invention, modification of the antibody to enhance ADCC and/or CDC of the antibody may be desirable. For example, antibodies with a mature carbohydrate structure that lacks fucose attached to an Fc region of the antibody are described in U.S. 2003/0157108 (Presta, L.). See also U.S. 2004/0093621 (Kyowa Hakko Kogyo Co., Ltd.). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof.

Thus a glycosylation variant optionally comprises an Fc region, wherein a carbohydrate structure attached to the Fc region lacks fucose. Such variants have improved ADCC function. Optionally, the Fc region further comprises one or more amino acid substitutions therein that further improve ADCC, for example, substitutions at positions 298, 333, and/or 334 of the Fc region (Eu numbering of residues). Examples of publications related to “defucosylated” or “fucose-deficient” antibodies include: U.S. 2003/0157108; WO 2000/61739; WO 2001/29246; U.S. 2003/0115614; U.S. 2002/0164328; U.S. 2004/0093621; U.S. 2004/0132140; U.S. 2004/0110704; U.S. 2004/0110282; U.S. 2004/0109865; WO 2003/085119; WO 2003/084570; WO 2005/035586; WO 2005/035778; Okazaki et al. J. Mol. Biol. 336:1239-1249 (2004); and Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); U.S. 2003/0157108, Presta, L; and WO 2004/056312, Adams et al., especially at Example 11), and knockout cell lines, such as alpha-1,6-fucosyltransferase gene, FUT8, -knockout CHO cells (Yamane-Ohnuki et al. Biotech. Bioeng. 87: 614 (2004)).

Modification of the antibody with respect to effector function, e.g. so as to enhance ADCC and/or CDC of the antibody, may be achieved by introducing one or more amino acid substitutions in an Fc region of an antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and ADCC. See Caron et al., J. Exp Med. 176:1191-1195 (1992) and Shopes, B. J. Immunol 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered that has dual Fc regions and may thereby have enhanced complement lysis and ADCC capabilities. See Stevenson et al. Anti-Cancer Drug Design 3:219-230 (1989). WO 2000/42072 (Presta, L.) describes antibodies with improved ADCC function in the presence of human effector cells, where the antibodies comprise amino acid substitutions in the Fc region thereof. Preferably, the antibody with improved ADCC comprises substitutions at positions 298, 333, and/or 334 of the Fc region. Preferably, the altered Fc region is a human IgG1 Fc region comprising or consisting of substitutions at one, two, or three of these positions.

Antibodies with altered C1q binding and/or CDC are described in WO 1999/51642 and U.S. Pat. Nos. 6,194,551, 6,242,195, 6,528,624, and 6,538,124 (Idusogie et al.). The antibodies comprise an amino acid substitution at one or more of amino acid positions 270, 322, 326, 327, 329, 313, 333, and/or 334 of the Fc region thereof. Non-depleting anti-CD4 antibodies comprising such amino acid substitutions constitute an embodiment of the invention.

To increase the serum half-life of the antibody, one may incorporate a salvage receptor binding epitope into the antibody (especially an antibody fragment) as described in U.S. Pat. No. 5,739,277, for example. As used herein, the term salvage receptor binding epitope refers to an epitope of the Fc region of an IgG molecule (e.g., IgG₁, IgG₂, IgG₃, or IgG₄) that is responsible for increasing the in vivo serum half-life of the IgG molecule. Antibodies with substitutions in an Fc region thereof and increased serum half-lives are also described in WO 2000/42072 (Presta, L.). Non-depleting anti-CD4 antibodies comprising such a salvage receptor binding epitope constitute an embodiment of the invention.

Any of the non-depleting (or other) antibodies of the invention may comprise at least one substitution in the Fc region that improves FcRn binding or serum half-life, e.g., a non-depleting anti-CD4 variant antibody. For example, the invention further provides an antibody comprising a variant Fc region with altered neonatal Fc receptor (FcRn) binding affinity. FcRn is structurally similar to major histocompatibility complex (MHC) and consists of an α-chain noncovalently bound to β2-microglobulin. The multiple functions of the neonatal Fc receptor FcRn are reviewed in Ghetie and Ward (2000) Annu. Rev. Immunol. 18:39-766. FcRn plays a role in the passive delivery of immunoglobulin IgGs from mother to young and the regulation of serum IgG levels. FcRn acts as a salvage receptor, binding and transporting pinocytosed IgGs in intact form both within and across cells, and rescuing them from a default degradative pathway. Although the mechanisms responsible for salvaging IgGs are still unclear, it is thought that unbound IgGs are directed toward proteolysis in lysosomes, whereas bound IgGs are recycled to the surface of the cells and released. This control takes place within the endothelial cells located throughout adult tissues. FcRn is expressed in at least the liver, mammary gland, and adult intestine. FcRn binds to IgG; the FcRn-IgG interaction has been studied extensively and appears to involve residues at the CH2, CH3 domain interface of the Fc region of IgG. These residues interact with residues primarily located in the α2 domain of FcRn.

In certain embodiments of the invention, a non-depleting anti-CD4 variant antibody may display increased binding to FcRn and comprise an amino acid modification at any one or more of amino acid positions 238, 256, 265, 272, 286, 303, 305, 307, 311, 312, 317, 340, 356, 360, 362, 376, 378, 380, 382, 413, 424 or 434 of the Fc region, wherein the numbering of the residues in the Fc region is that of the EU index as in Kabat. See, e.g., U.S. Pat. No. 6,737,056; and, Shields et al., J. Biol. Chem. 276: 6591-6604 (2001). In one embodiment of the invention, an antibody comprises a variant IgG Fc region comprising at least an amino acid substitution at Asn 434 to His (N434H). In one embodiment of the invention, an antibody comprises a variant IgG Fc region comprising at least an amino acid substitution at Asn 434 to Ala (N434A). Typically, these variants comprise a higher binding affinity for FcRN than polypeptides having native sequence/wild type sequence Fc region. These Fc variant polypeptide and antibodies have the advantage of being salvaged and recycled rather than degraded. These non-depleting anti-CD4 variant antibodies can be used in the methods provided herein. As just one example of a non-depleting CD4 variant antibody, any of the TRX1 antibodies described herein can include a substitution at heavy-chain position 434, such as N434A or N434H.

Serum half-life of the antibody may also be increased by incorporation of a serum albumin binding peptide into the antibody as disclosed in U.S. application Serial No. 20040001827 (Dennis, M.). Non-depleting anti-CD4 antibodies comprising such serum albumin binding peptides constitute an embodiment of the invention.

Engineered antibodies with three or more (preferably four) functional antigen-binding sites are also contemplated (US 2002/0004587 A1, Miller et al.). Non-depleting anti-CD4 antibodies comprising such multiple antigen-binding sites constitute an embodiment of the invention.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, New York, 2000 and Current Protocols in Molecular Biology, F. M. Ausubel et al., eds., Current Protocols, a joint venture between Greene Publishing Associates, Inc. and John Wiley & Sons, Inc., (supplemented through 2006). Other useful references, e.g. for cell isolation and culture (e.g., for subsequent nucleic acid or protein isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla. Methods of making nucleic acids (e.g., by in vitro amplification, purification from cells, or chemical synthesis), methods for manipulating nucleic acids (e.g., site-directed mutagenesis, by restriction enzyme digestion, ligation, etc.), and various vectors, cell lines and the like useful in manipulating and making nucleic acids are described in the above references. In addition, essentially any polynucleotide (including, e.g., labeled or biotinylated polynucleotides) can be custom or standard ordered from any of a variety of commercial sources.

Administration

As will be understood by those of ordinary skill in the art, the appropriate doses of non-depleting CD4 antibodies will be generally around those already employed in clinical therapies wherein similar antibodies are administered alone or in combination with other therapeutics. Variation in dosage will likely occur depending on the condition being treated. The physician administering treatment will be able to determine the appropriate dose for the individual subject. Preparation and dosing schedules for commercially available second compounds administered in combination with the non-depleting CD4 antibodies may be used according to manufacturers' instructions or determined empirically by the skilled practitioner.

For the prevention or treatment of disease, the appropriate dosage of the antibody and any second compound administered in combination with the non-depleting antibody will depend on the type of disease to be treated, as defined above, the severity and course of the disease, whether the non-depleting antibody or combination is administered for preventive or therapeutic purposes, previous therapy, the patient's clinical history and response to the antibody or combination, and the discretion of the attending physician. The non-depleting antibody or combination is suitably administered to the patient at one time or more typically over a series of treatments.

Depending on the type and severity of the disease, about 1 μg/kg to 50 mg/kg (e.g. 0.1-20 mg/kg) of non-depleting CD4 antibody is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. Typically, the clinician will administer an antibody (alone or in combination with a second compound) of the invention until a dosage(s) is reached that provides the required biological effect. The progress of the therapy of the invention is easily monitored by conventional techniques and assays.

For example, a TRX1 non-depleting CD4 antibody is optionally administered as described above or in U.S. patent application publication 2003/0108518 or 2003/0219403. In one embodiment, 3-5 mg/kg (mg of antibody per kg body weight of the subject) is administered to the subject, alone or in combination with a second compound as described herein, and treatment is sustained until a desired suppression of disease symptoms occurs. The non-depleting antibody is optionally administered over a period of time in order to maintain in the subject appropriate levels of antibody (or if the antibody is used in combination with a second compound, appropriate levels of the combination of the antibody and second compound) to achieve and maintain suppression of symptoms.

The non-depleting CD4 antibody can be administered by any suitable means, including parenteral, topical, subcutaneous, intraperitoneal, intrapulmonary, intranasal, and/or intralesional administration. Parenteral infusions include intramuscular, intravenous, intraarterial, intraperitoneal, or subcutaneous administration. Intrathecal administration is also contemplated (see, e.g., U.S. patent application publication 2002/0009444 by Grillo-Lopez). In addition, the antibody may suitably be administered by pulse infusion, e.g., with declining doses of the antibody. Preferably, the dosing is given intravenously or subcutaneously, and optionally by intravenous infusion(s). Each exposure may be provided using the same or a different administration means. In one embodiment, each exposure is by intravenous administration.

As noted, the non-depleting CD4 antibody can be administered alone or in combination with at least a second compound. These second compounds are generally used in the same dosages and with administration routes as used heretofore, or about from 1 to 99% of the heretofore-employed dosages. If such second compounds are used, preferably they are used in lower amounts than if the non-depleting CD4 antibody were not present, so as to eliminate or reduce side effects caused thereby.

Also as noted, a variety of suitable second compounds are known in the art, and dosages and administration methods for such second compounds have likewise been described. As just one example, the non-depleting CD4 antibody can be administered in combination with cyclophosphamide for treatment of lupus (or MS, rheumatoid arthritis, or inflammatory bowel disease, or other disorder as described herein). A variety of cyclophosphamide treatment regimens have been described in the literature. Exemplary regimens include, but are not limited to, intravenous administration of 0.5-1.0 g/m² monthly for six months than every three months out to 30 months; and intravenous administration of 500 mg every two weeks for three months; oral administration of 1-3 mg/kg per day for twelve weeks or six months. See, e.g., Petri (2004) “Cyclosphosphamide: new approaches for systemic lupus erythematosus” Lupus 13:366-371 and Petri and Brodsky (2006) “High-dose cyclophosphamide and stem cell transplantation for refractory systemic lupus erythematosus” JAMA 295:559-560.

The administration of the non-depleting anti-CD4 antibody and any second compound of the invention can be done simultaneously, e.g., as a single composition or as two or more distinct compositions using the same or different administration routes. Alternatively, or additionally, the administration can be done sequentially, in any order. In certain embodiments, intervals ranging from minutes to days, to weeks to months, can be present between the administrations of the two or more compositions. For example, the non-depleting anti-CD4 antibody may be administered first, followed by the second compound of the invention. However, simultaneous administration or administration of the second compound of the invention first is also contemplated.

As noted above, a third, fourth, etc. compound is optionally administered in combination with the non-depleting CD4 antibody and the second compound. Similarly, treatment for symptoms secondary or related to lupus (e.g., spasticity, incontinence, pain, fatigue) or MS, rheumatoid arthritis, inflammatory bowel disease, or other condition or disease can be administered to the subject, e.g., during treatment with the non-depleting CD4 antibody or combination.

### Pharmaceutical Formulations

Therapeutic formulations of the antibodies used in accordance with the present invention are prepared for storage by mixing a non-depleting CD4 antibody having the desired degree of purity with optional pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as Tween®, Pluronics®, or PEG.

Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein. Crystallized forms of the antibody are also contemplated. See, for example, U.S. 2002/0136719A1 (Shenoy et al.).

The formulation herein may also contain at least a second compound as necessary for the particular indication being treated, preferably those with complementary activities that do not adversely affect each other. For example, it may be desirable to further provide a cytotoxic agent (e.g. methotrexate, cyclophosphamide, or azathioprine), chemotherapeutic agent, immunosuppressive agent, cytokine, cytokine antagonist or antibody, growth factor, hormone, integrin, integrin antagonist or antibody (e.g., an LFA-1 antibody, or an alpha 4 integrin antibody such as natalizumab), interferon class drug such as IFN-beta-1a or IFN-beta-1b, an oligopeptide such as glatiramer acetate, intravenous immunoglobulin (gamma globulin), lymphocyte-depleting drug (e.g., mitoxantrone, cyclophosphamide, CamPath® antibodies, or cladribine), non-lymphocyte-depleting immunosuppressive drug (e.g., MMF or cyclosporine), cholesterol-lowering drug of the “statin” class, estradiol, drug that treats symptoms secondary or related to lupus, MS, rheumatoid arthritis, or inflammatory bowel disease (e.g., spasticity, incontinence, pain, fatigue), a TNF inhibitor, DMARD, NSAID, corticosteroid (e.g., methylprednisolone, prednisone, dexamethasone, or glucorticoid), levothyroxine, cyclosporin A, somatastatin analogue, anti-metabolite, a T- or B-cell surface antagonist/antibody, etc., or others as noted above in the formulation. The type and effective amounts of such other agents depend, for example, on the amount of antibody present in the formulation, the type of lupus or MS or other condition or disease being treated, and clinical parameters of the subjects.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug-delivery systems (for example, liposomes, albumin microspheres, microemulsions, nano-particles and nanocapsules) or in macroemulsions. Such techniques are disclosed, e.g., in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the non-depleting antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### Articles of Manufacture

In another embodiment of the invention, an article of manufacture containing materials useful for the treatment of lupus, MS, rheumatoid arthritis, inflammatory bowel disease, or other condition or disease described above is provided. Preferably, the article of manufacture comprises (a) a container comprising a composition comprising a non-depleting CD4 antibody and a pharmaceutically acceptable carrier or diluent within the container; and (b) a package insert with instructions for treating lupus, MS, rheumatoid arthritis, inflammatory bowel disease, or other condition or disease in a subject by administration of the antibody, alone or in combination with at least a second compound.

The package insert is on or associated with the container. Suitable containers include, for example, bottles, vials, syringes, etc. The containers may be formed from a variety of materials such as glass or plastic. The container holds or contains a composition that is effective for treating the lupus, MS, rheumatoid arthritis, inflammatory bowel disease, or other condition or disease and may have a sterile access port (for example, the container may be an intravenous solution bag or a vial having a stopper pierceable by a hypodermic injection needle). At least one active agent in the composition is the non-depleting antibody. The label or package insert indicates that the composition is used for treating lupus, MS, rheumatoid arthritis, inflammatory bowel disease, or other condition or disease in a subject eligible for treatment with specific guidance regarding dosing amounts and intervals of antibody and any other drug being provided.

The article of manufacture may further comprise a second container comprising a pharmaceutically acceptable diluent buffer, such as bacteriostatic water for injection (BWFI), phosphate-buffered saline, Ringer's solution, and dextrose solution. The article of manufacture optionally comprises a second or third container comprising a second compound, such as any of those described herein, where the article further comprises instructions on the package insert for treating the subject with the second compound. Alternatively, the composition comprising the non-depleting CD4 antibody can also comprise the second compound. The article of manufacture may further include other materials desirable from a commercial and user standpoint, including other buffers, diluents, filters, needles, and syringes.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Treatment of Lupus with Non-Depleting CD4 Antibody, Alone and in Combination

The following sets forth a series of experiments that demonstrate that a non-depleting CD4 antibody is efficacious in a preclinical model of SLE. Performance of the antibody is compared to that of exemplary standard of care and experimental treatments.

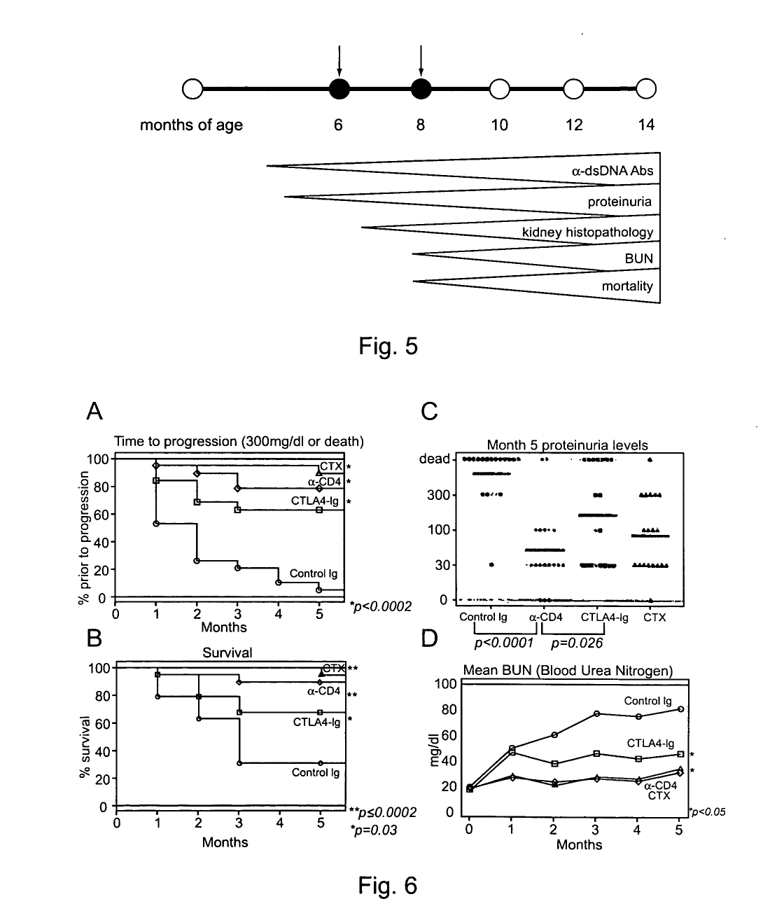

NZBxW F1 mice exhibit spontaneous lupus-like kidney disease, providing a useful preclinical efficacy model of SLE (see, e.g., Theofilopoulos (1992) “Murine models of systemic lupus erythematosus” in Systemic Lupus Erythematosus, Lahita (ed.) Churchill Livingstone, New York, 121-194). FIG. 5 schematically illustrates progression of the disease by age in this model. Symptoms observed include the appearance of ds-DNA antibodies, proteinuria, kidney histopathology, increased blood urea nitrogen (BUN), and increased mortality. Arrows indicate the time points at which treatment with the non-depleting CD4 antibody was initiated in two studies comparing the antibody with other treatments.

In this model, preclinical efficacy of rat non-depleting CD4 antibody YTS177 (Cobbold et al. (1990) “The induction of skingraft tolerance in MHC-mismatched or primed recipients: primed T-cells can be tolerized in the periphery with CD4 and CD8 antibodies” Eur J Immunol 20:2747-2755) was compared to that of a non-binding control antibody (control Ab or control Ig), CTLA4-Ig (in clinical development), and cyclophosphamide (Cytoxan®, CTX; a current standard of care treatment). The YTS177 non-depleting CD4 antibody was a gift from Herman Waldmann, Oxford. The control antibody was an irrelevant mouse IgG1 antibody (a mouse antibody was used for the control since an irrelevant rat antibody would elicit an immune response against itself, influencing the course of disease; the rat anti-CD4 antibody prevents such a response to itself). The CTLA4-Ig construct used includes the extracellular domain of murine CTLA-4 fused to human IgG1 hinge -C3, C4 Ig domains and is modeled after Linsley et al. (1991) J Exp Med 174(3):561.

At 8 months of age, NZB×NZW mice were screened for proteinuria and randomized into 5 groups based on their proteinuria scores. At this age, disease is considered to be moderate-severe. At the onset of the experiment, each group of 19 mice was composed of the following distribution of protein concentrations in the urine: 32% at >300 mg/dl; 24% at 100-300 mg/dl; and 44% at 30-100 mg/dl. Mice were treated continuously for 6 months with either control antibody (control Ab or control Ig), YTS177 (non-depleting anti-CD4), CTLA4-Ig, cyclophosphamide (CTX), or a combination of anti-CD4 and CTX. YTS177 and CTLA4-Ig were delivered 3×/week at 5 mg/kg by intraperitoneal (IP) injection; cyclophosphamide (CTX) was given IP at 50 mg/kg every 10 days (alone or in combination with the indicated amount of YTS177). Mice were monitored for changes in urine protein concentration (e.g., proteinuria), Blood Urea Nitrogen (BUN), and survival.

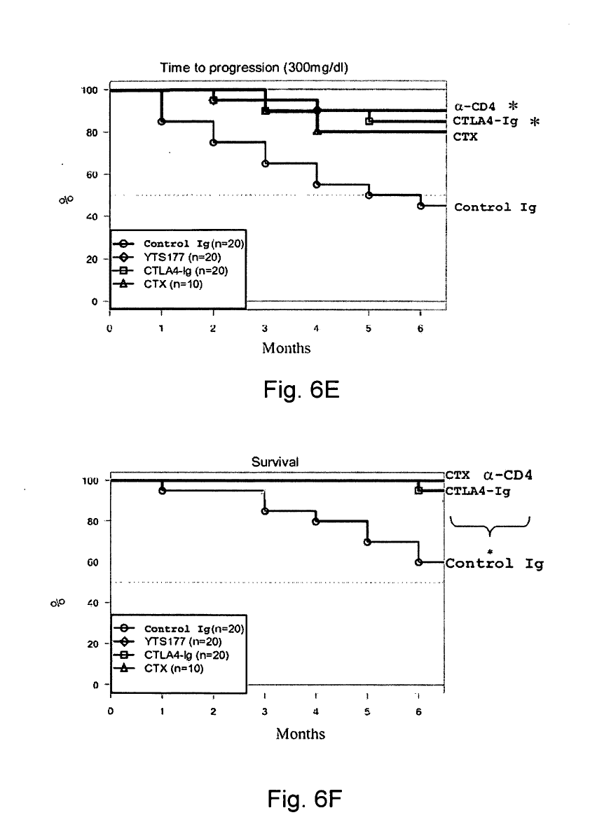

As shown in FIG. 6, administration of the non-depleting CD4 antibody delayed time to progression (FIG. 6A), increased survival (FIG. 6B), decreased proteinuria (data for month 5 after treatment are shown, FIG. 6C), and decreased mean BUN (FIG. 6D).

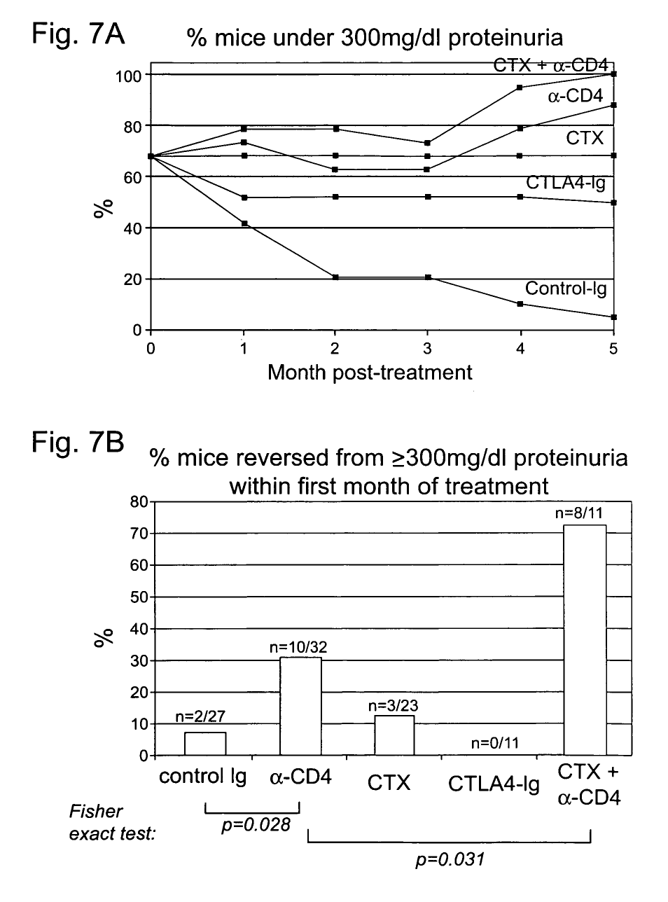

Treatment with the non-depleting CD4 antibody can reverse severe lupus nephritis, as shown in FIG. 7. FIG. 7A illustrates the percentage of mice under 300 mg/dl proteinuria at the indicated times after treatment. Administration of the non-depleting CD4 antibody alone or in combination with cyclophosphamide resulted in a net decrease in mice exhibiting >300 mg/dl proteinuria, indicating a reversal of nephritis symptoms in very late stage disease that was not observed in groups treated with the control antibody, CTLA4-Ig, or cyclophosphamide alone. FIG. 7B shows the percentage of mice reversed from 300 mg/dl proteinuria within the first month of treatment. (FIG. 7B illustrates data compiled from four studies including the one described herein and three similar studies. Data include only mice whose proteinuria was >300 mg/dl at time of treatment onset.) A synergistic effect in the capacity to reverse proteinuria was seen when the CD4 antibody was combined with cyclophosphamide (CTX).

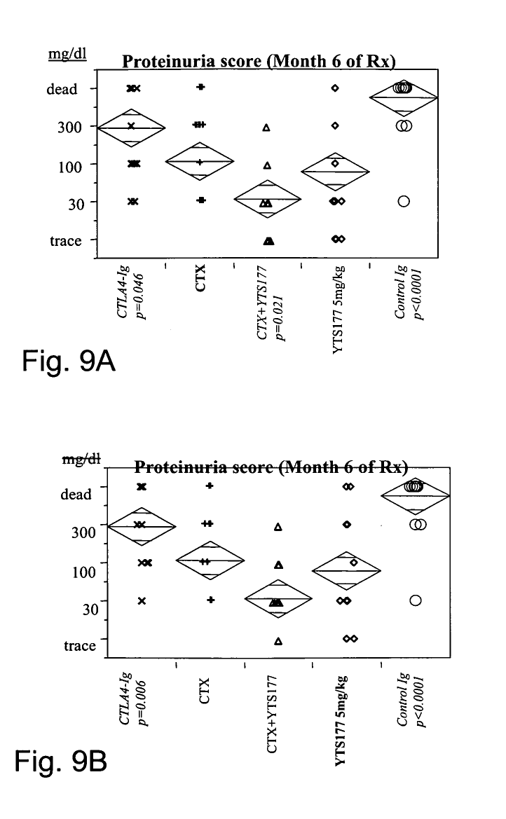

Treatment with a combination of the non-depleting CD4 antibody and cyclophosphamide is also effective in decreasing proteinuria. FIG. 9 illustrates multiple comparison analysis of proteinuria at month 6 of treatment, using Dunnett's method with the cyclophosphamide treated group as the reference control group in FIG. 9A and the non-depleting CD4 antibody treated group as the reference control group in FIG. 9B. The reference controls are designated in bold, and only p values for groups that achieve statistical significance vs. the reference control are designated on the graph. The results again demonstrate that the non-depleting CD4 antibody was superior to CTLA4-Ig in decreasing proteinuria (see, e.g., FIG. 9B). The results also demonstrate that the combination of the non-depleting CD4 antibody and cyclophosphamide provided significant benefit over cyclophosphamide alone in decreasing proteinuria in the model (see, e.g., FIG. 9A).

Examination of kidney sections stained for CD4 and CD8 revealed lymphocytic infiltrates in the renal medullary or pelvic interstitium in mice after four months of treatment with control antibody. Treatment with the CD4 antibody or with CTLA4-Ig, on the other hand, resulted in a reduction in CD4+ cells observed in the kidney interstitium at four months post-treatment. CD4 antibody treatment did not impact the number of CD8+ T cells observed in the kidney.

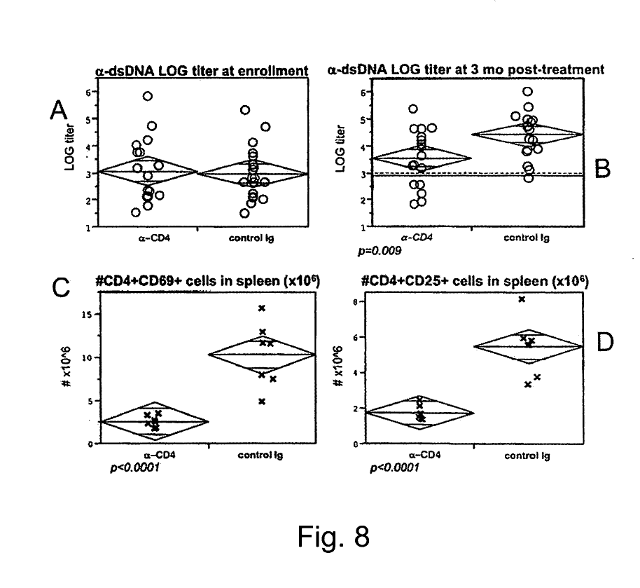

Treatment of NZBxW F1 mice exhibiting spontaneous lupus-like kidney disease (SLE mouse model) with the non-depleting CD4 antibody also limited increases in ds-DNA antibody titers. As shown in FIG. 8, increases in ds-DNA antibody titers over time were less in animals treated within the non-depleting CD4 antibody as compared to animals treated with the control antibody. Compare FIG. 8A, showing titer at enrollment (an approximate average of 3 logs for each of the treatment groups), with FIG. 8B, showing titer three months post-treatment (approximately 3.5 logs and 4.5 logs for the non-depleting anti-CD4 and control antibody treatment groups, respectively). In this experiment, treatment was initiated at six months of age rather than eight months of age.

In addition, treatment with the CD4 antibody decreased the number of activated CD4+ T cells found in the spleen, as determined by flow cytometry with antibodies directed against surface proteins associated with T cell activation. As shown in FIG. 8, the number of both CD4+CD69+ cells (FIG. 8C) and CD4+CD25+ cells (FIG. 8D) found in spleen three weeks post-treatment was less in non-depleting CD4 antibody treated animals as compared to control antibody treated animals (treatment initiated at eight months of age).

Treatment with the non-depleting CD4 antibody was also effective when introduced in mild disease rather than moderate-severe disease. NZB×NZW mice at six months of age, all at 30-100 mg/dl proteinuria, were treated with control Ab, YTS177 (non-depleting anti-CD4), CTLA4-Ig, or cyclophosphamide (Cytoxan®) basically as described above. Mice were monitored for changes in proteinuria and survival. As shown in FIG. 6, administration of the non-depleting CD4 antibody beginning at six months of age delayed time to progression (FIG. 6E) and increased survival (FIG. 6F) relative to control, demonstrating that the non-depleting CD4 antibody is highly effective when introduced in mild disease. (All treatments are very effective when compared to control: at 7 months time to progression *p<0.025 (FIG. 6E) and survival *p<0.04 (FIG. 6F).)

In summary, treatment with the non-depleting CD4 antibody was efficacious NZBxW F1 mice when introduced early or late in disease. Treatment with the antibody extended disease-free progression and survival, delayed elevation of BUN and development of glomerulonephritis, limited increases in anti-dsDNA titers, and decreased activated CD4+ T cell numbers. The effect observed with the antibody at 5 or 6 months of treatment was comparable to that of cyclophosphamide and superior to that of CTLA4-Ig in reducing proteinuria; the distinction between anti-CD4 and CTLA4-Ig was more evident in late disease. In addition, combining the non-depleting CD4 antibody with cyclophosphamide provided significant benefit over cyclophosphamide alone in the NZB/W F1 model of SLE.

### Experimental Procedures

Urinalysis

Proteinuria was measured using a Clinitek® 50 Urine Chemistry Analyzer (Bayer Corporation, Elkhart, Ind., USA). A drop of freshly collected urine was placed on a reagent strip (Multistix® 10 SG, Bayer), and the strip was immediately inserted into the analyzer after removal of excess urine by blotting with a clean gauze sponge.

Measurement of Blood Urea Nitrogen Levels

Blood urea nitrogen was measured using a Cobas Integra® 400 chemistry analyzer (Roche Diagnostics, Basel, Switzerland) and urea detection reagent (also supplied by Roche Diagnostics) according to the manufacturer's instructions. Precinorm™ and Precipath™ lyophilized human serum controls (Roche Diagnostics) were used as normal and abnormal controls, respectively.

Staining of Kidney Sections for CD4 and CD8

For CD4/CD8 dual labeled immunohistochemistry, 5 micron thick frozen sections of kidney were cut and fixed in ice cold acetone (−20° C.) for 5 minutes, rinsed 2×5 minutes in TBS/0.1% Tween 20 (TBST), and then blocked for endogenous peroxidase activity with glucose oxidase for 1 hour at 37° C. Sections were then rinsed in TBST and blocked for endogenous avidin/biotin using an avidin/biotin blocking kit from Vector Labs (Vector Labs, Burlingame, Calif.). After further rinsing in TBST, endogenous immunoglobulins were blocked with 10% rabbit serum/3% BSA/TBS for 30 minutes at room temperature (RT).

For CD8 labeling, sections were incubated with biotinylated rat anti-mouse CD8 monoclonal antibody (MAb), clone 53.6-7 (Pharmingen, San Diego, Calif.), at 8 ug/ml for 1 hour at RT. For the negative control, a naïve isotype, rat IgG2a, was used as the primary anti-sera. After rinsing in TBST, sections were incubated in Vectastain ABC-Elite reagent (Vector Labs) for 30 minutes at RT. The staining reaction was then visualized using metal enhanced DAB as the chromogen (Pierce Biotechnology, Rockford, Ill.).

For secondary labeling with CD4 antibody, sections were once again blocked for avidin/biotin (from the first reaction) using the Vector Labs avidin/biotin blocking kit. Sections were then incubated with a rat anti-mouse CD4 MAb, clone RM4-4 (Pharmingen) at 0.5 ug/ml for 1 hour at RT. For the negative control, a naïve isotype, rat IgG2b, was used as the primary anti-sera. After rinsing in TBST, sections were then incubated with streptavidin-HRP complex from a TSA™ (tyramide signal amplification) kit (Perkin-Elmer LAS Inc., Boston Mass.) for 30 minutes at RT. After rinsing in TBST, sections were then incubated with biotinylated TSA™ amplification reagent (Perkin-Elmer LAS Inc) for 3 minutes at RT followed by a second round of streptavidin-HRP for 30 minutes at RT. The staining reaction was then visualized using Vector® Red (Vector Labs) as the chromogen.

Dual labeled sections were then lightly counterstained with Myer's hematoxylin for 1 minute, rinsed in tap water and coverslipped using Crystal/Mount (Biomeda Corporation, Foster City, Calif.).

Determination of Double Stranded DNA Antibody Titers

Anti ds-DNA antibody titers were determined by ELISA. Nunc MAXIsorb immunoplate 384-well plates (number 464718) were coated with poly-L-lysine (25 μl per well, 0.01%, Sigma P4707) for 1 hr at RT, washed with deionized water, air dried at RT for 1 hr, and then coated with calf thymus DNA (Sigma D1501, 25 μl per well, 2.5 μg/ml in PBS) at 4° C. overnight. The calf thymus DNA solution was decanted from the plate, 50 μl of blocking buffer (PBS, 0.5% BSA pH7.2) was added, and the plate was shaken for 1 hr at RT. The plate was then washed three times with washing buffer (PBS, 0.05% Tween™ 20 (polyoxyethylene(20)sorbitan monolaurate), pH7.2).

Serial dilutions of serum samples in assay buffer (PBS, 0.5% BSA, 0.05% Tween™ 20, 0.01% Procline 3000) were prepared; an initial 25-fold dilution was followed by serial 3-fold dilutions performed with a Precision 2000™ automated pipetting system. Serial dilutions of negative control serum (a pool of mouse serum with a low or background anti-dsDNA antibody level) were prepared in the same manner. One or more dilutions of a positive control serum are optionally also prepared (e.g., a 5000 fold dilution of NZB F1 serum).

Diluted serum samples were added to the washed plate, e.g., using a rapid plate robot to add 25 ul of diluted serum. The plate was incubated for 2 hr at RT with gentle agitation, then washed six times with washing buffer. HRP (horseradish peroxidase)-conjugated anti-mouse Fc antibody was added to each well (25 μl of anti-mu-FcHRP from Jackson ImmunoResearch Laboratories, Inc., catalog number 115-035-071, diluted 5000-fold in assay buffer), and the plate was incubated at RT for 1 hr with gentle agitation. Substrate solution (25 μl per well; one part TMB substrate plus one part Peroxidase Solution B, both obtained from Kirkegaard & Perry) was added, and color was developed. Stop solution was added (25 μl per well of 1M H₃PO₄), and the plate was read at 450/620 nm.

Anti ds-DNA antibody titers for the serum samples were calculated using the following formula:

$$Titer = Log [ ( \frac{{HighA}_{450 / 620} - CP}{{HighA}_{450 / 620} - {LowA}_{450 / 620}} ) ( DF 1 - DF 2 ) + DF 2 ]$$

where CP (the cut point) is 3 times the absorbance of the negative control serum mean; High A_{450/620} is the absorbance (A_{450/620}) which is closest to but higher in value than the cut point; Low A_{450/620} is the absorbance (A_{450/620}) which is closest to but lower in value than the cut point; DF1 is the dilution factor of the low A_{450/620} value, closest to but lower in value than the cut point; and DF2 is the dilution factor of the high A_{450/620} value, closest to but higher in value than the cut point.

Flow Cytometry

Numbers of activated CD4+ T cells found in spleen were determined by flow cytometry as follows. Whole spleens were harvested and crushed into single cell suspensions, which were then red blood cell lysed using EL buffer (erythrocyte lysis buffer, from Qiagen, Valencia, Calif., catalog number 79217), passed through a 70 micron cell strainer, and then resuspended for cell counts. A fixed volume of each cell suspension was mixed with a fluorescent bead (Polysciences, Inc., catalog number 18862) solution of known concentration. The mixture was then run on a FACScan™ flow cytometer from BD Biosciences (Franklin Lakes, N.J.). By collecting a fixed number of beads for each mixture, the total number of live cells could be calculated and subsequently used to determine total numbers of cell subpopulations for the spleen of each mouse after further FACS analysis.

To 1×10⁶ cells, a saturating amount of fluorophore-conjugated antibodies were added and incubated on ice for 30 minutes, followed by washing with cold buffer. Spleen cells were stained with anti-CD4 (BD Pharmingen, catalog number 553055, clone RM4-4), anti-CD3 (BD Pharmingen, catalog number 555276, clone 17A2), and anti-CD69 (BD Pharmingen, catalog number 553237, clone H1.2F3) or with anti-CD4, anti-CD3, and anti-CD25 (Miltenyi Biotec, catalog number 130-091-013). CD3 staining facilitated separation of CD4 and CD8 T cells, since CD8 cells are positive for CD3 but negative for CD4. Samples were analyzed by flow cytometry on a FACSCalibur™ flow cytometer from BD Biosciences.

### Example 2

### Treatment of Multiple Sclerosis with Non-Depleting CD4 Antibody

The following sets forth a series of experiments that demonstrate that a non-depleting CD4 antibody is efficacious in a preclinical model of MS. Performance of the antibody is compared to that of exemplary standards of care and experimental treatments.

Experimental autoimmune encephalomyelitis (EAE) is an inflammatory condition of the central nervous system (CNS) with similarities to MS; in both diseases, demyelination results in impaired nerve conduction and paralysis. Relapsing and remitting EAE induced by injection of proteolipid protein (PLP) peptide in SJL/J mice provides a useful preclinical efficacy model of MS (see, e.g., Miller and Karpus (1996) “Experimental Autoimmune Encephalomyelitis in the Mouse” in Current Protocols in Immunology, Coligan et al. (eds.), John Wiley & Sons, Inc. and Sobel et al. (1990) “Acute experimental allergic encephalomyelitis in SJL/J mice induced by a synthetic peptide of myelin proteolipid protein” J Neuropathol Exp Neurol. 49(5):468-79).

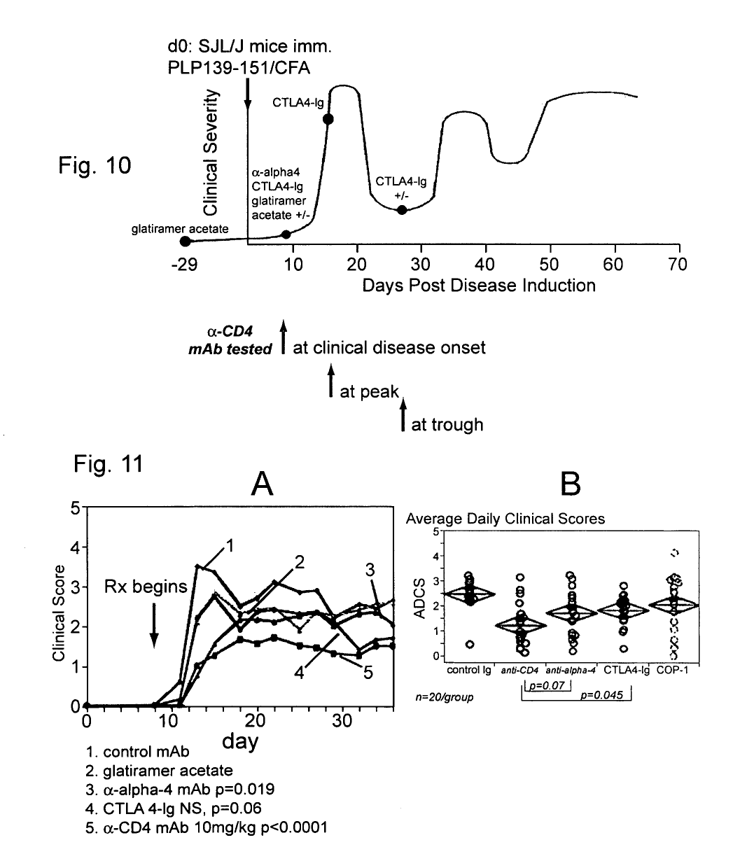

FIG. 10 schematically illustrates progression of the disease over time after injection of the PLP peptide in this model. Injection at day 0 is followed by disease onset (days 0-15), remission (days 15-25), and relapse (day 25-termination of the study at days 60-70). Standardized clinical neurological scores are assigned as follows: 0— no disease; 1—limp tail or hind limb weakness, but not both; 2—limp tail and hind limb weakness; 3—partial hind limb paralysis; 4—complete hind limb paralysis; and 5—moribund state, death by EAE, sacrifice for humane reasons. In the schematic, arrows indicate the time points at which treatment with the non-depleting CD4 antibody was initiated in studies comparing the antibody with other treatments. Dots indicate the time points at which other treatments have been previously shown to be effective.

In this model, preclinical efficacy of non-depleting CD4 antibody was compared to that of a control antibody (described above), CTLA4-Ig, an alpha-4 integrin antibody, and glatiramer acetate (Copaxone®). SJL/J mice were immunized on Day 0 with the PLP-139-151 peptide in CFA (complete Freunds adjuvant). Mice were screened 3×/week for disease scores, as noted above; at terminal endpoints, histopathology (brain and spinal cord) was examined. If therapy began after disease onset, mice were monitored for disease scores, then randomized into groups with comparable disease scores prior to treatment. In three separate studies, antibody (or other) treatment began during disease onset at day 8, at peak of disease at day 14, or in the trough on day 24. The non-depleting CD4 antibody, the control antibody, CTLA4-Ig, the alpha-4 integrin antibody, and glatiramer acetate were delivered 3×/week at 10 mg/kg.

Except where indicated, in these experiments, the non-depleting CD4 antibody used was a murinized YTS177 antibody. Murinized YTS177 included the heavy and light chain variable regions from the rat YTS177 antibody, cloned upstream of mouse IgG2a heavy chain and kappa light chain constant sequences. The heavy chain included 2 single amino acid substitutions in the Fc receptor binding region (residues corresponding to human IgG1 residues D265 and N297 have been changed to alanine).

As illustrated in FIG. 11, the non-depleting CD4 antibody was superior to CTLA4-Ig and glatiramer acetate when introduced at disease onset (treatment initiated at day 8). FIG. 11A presents a graph of the clinical score over time for groups treated with the control antibody, glatiramer acetate, the alpha-4 integrin antibody, CTLA4-Ig, and the CD4 antibody. FIG. 11B presents the average daily clinical scores for these groups.

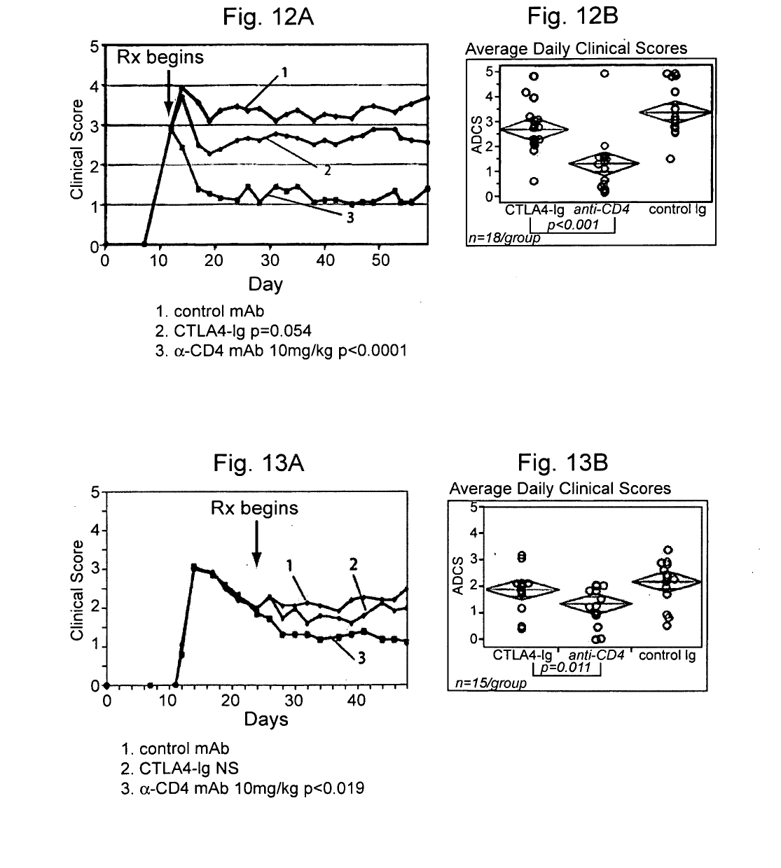

The CD4 antibody was also superior to CTLA4-Ig when introduced at the peak of disease (treatment initiated at day 14), as illustrated in FIG. 12. FIG. 12A presents a graph of the clinical score over time for groups treated with the control antibody, CTLA4-Ig, and the CD4 antibody. (glatiramer acetate and the alpha-4 integrin antibody are ineffective at this time point.) FIG. 12B presents the average daily clinical scores for these groups. The effect observed with the CD4 antibody is representative of three independent experiments.

As illustrated in FIG. 13, the CD4 antibody was also superior to CTLA4-Ig when introduced late in disease (treatment initiated at day 24). FIG. 13A presents a graph of the clinical score over time for groups treated with the control antibody, CTLA4-Ig, and the CD4 antibody. FIG. 13B presents the average daily clinical scores for these groups. The effect observed with the non-depleting CD4 antibody is representative of two independent experiments.

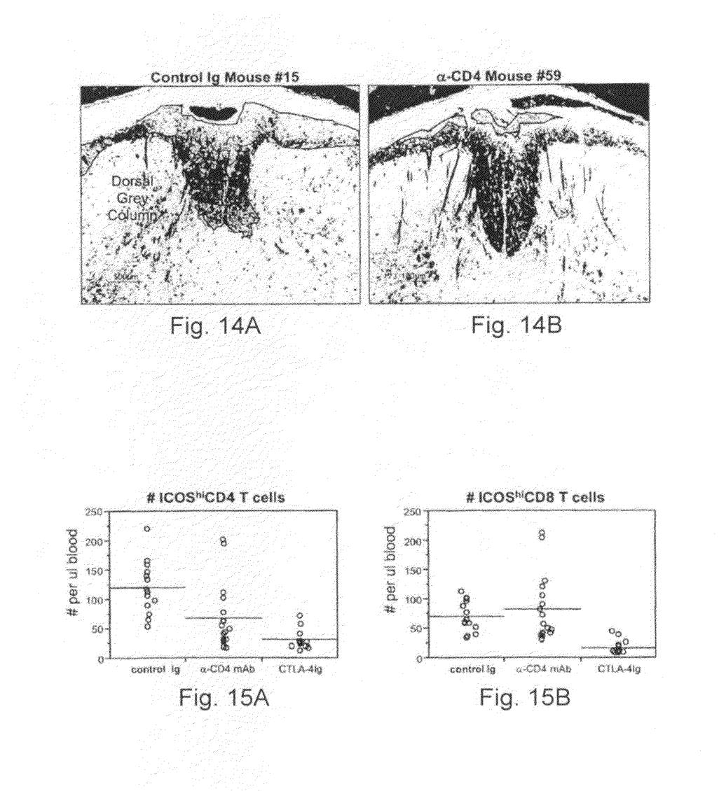

Treatment with a non-depleting CD4 antibody decreased demyelination in EAE, as shown in FIG. 14. Treatment with the antibody (YTS177 rather than murinized YTS177 in this experiment) began near the peak of the acute phase of disease (day 12) and was continued to termination of the study at day 80. Spinal chords were harvested, fixed, and stained with Luxol Fast Blue stain (which stains intact myelin dark blue). The outlined areas depict areas of demyelination. Selected mice are representative of the mean average demyelination score per group.

Treatment with the CD4 antibody (murinized YTS177) also reduced CD4+ T cell infiltrate in the relapsing/remitting EAE model. For example, in spinal cord sections taken from animals at days 60 after initiation of treatment at day 14 and stained for CD4 and CD8 as described above in Example 1, CD4+ but not CD8+ infiltrate was reduced in CD4 antibody treated animals as compared with control antibody treated animals.

Mice treated with the non-depleting CD4 antibody remained immunocompetent, showing normal survival following Listeria infection. For example, at day 8 following Listeria infection, 10/10 animals treated with the non-depleting CD4 antibody (murinized YTS177) survived, compared with 8/10 animals treated with the control Ig antibody, 3/10 animals treated with CTLA4-Ig, and 0/10 animals treated with TNFRII-Fc (Wooley et al. (1993) J of Immunol 151(11):6602). Treatments began one day prior to inoculation with Listeria with an initial dose of 20 mg/kg of the therapeutics, after which all therapeutics were dosed at 5 mg/kg 3×/week for duration of the study.

As shown in FIG. 15, treatment with the CD4 antibody selectively reduced CD4+ effector/memory cells in the blood. The number of ICOS^{hi}CD4 or ICOS^{hi}CD8 T cells per μl of blood as determined by flow cytometry is shown for animals treated with the control antibody, the CD4 antibody, or CTLA4-Ig. (ICOS^{hi} is a marker of effector/memory T cells, representing less than 4% of T cells in blood of normal mice and seen to increase upon EAE development to approximately 15-20%.) Unlike CTLA4-Ig, the non-depleting CD4 antibody decreased the number of CD4+ cells without decrease in the number of CD8+ cells. In this experiment, treatment was initiated at day 14; cells were counted at day 46.

In summary, treatment with non-depleting CD4 antibody is efficacious in the SJL/J model of relapsing/remitting EAE. Treatment with the antibody decreased clinical scores at all time points of intervention, decreased histology scores in brain and spinal cord, decreased CD4+ but not CD8+ infiltrate in CNS, and decreased ICOS^{hi} CD4+ but not CD8+ T cell numbers. The efficacy of the CD4 antibody was superior to that of CTLA4-Ig and glatiramer acetate, and at least matched that of the alpha-4 integrin monoclonal antibody.

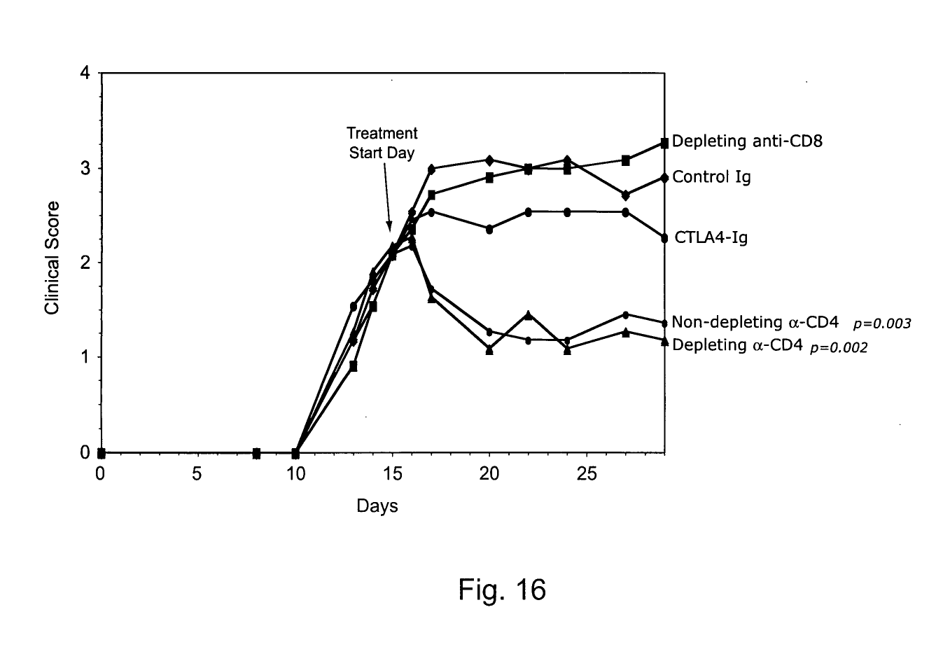

Treatment with CD4 antibody is also effective in a different MS model, MOG-peptide induced EAE in C57Blk6 mice. The MOG model does not show periodic remissions and is thus more an acute/chronic model of MS. A rapid reversal in neurological symptoms was observed in the MOG model, similar to that observed in the SJL/J model, when treatment began near the peak of the disease. As shown in FIG. 16, treatment with a non-depleting (or a depleting) CD4 antibody decreased clinical score as compared to treatment with control antibody, CTLA4-Ig, or a depleting CD8 antibody.

### Experimental Procedures

Flow Cytometry

Numbers of effector/memory cells in the blood were determined by flow cytometry as follows. A fixed volume of blood was collected retro-orbitally into heparinized tubes, then red blood cell lysed, and resuspended for cell counts. A fixed volume of each cell suspension was mixed with a fluorescent bead solution of known concentration for determination of total numbers of cell subpopulations for the blood of each mouse, as described above in Example 1.

To 1×10⁶ cells, a saturating amount of fluorophore-conjugated antibodies were added and incubated on ice for 30 minutes, followed by washing with cold buffer. Blood cells were stained with anti-CD4 (BD Pharmingen, catalog number 553055, clone RM4-4), anti-CD8a (BD Pharmingen, catalog number 553033, clone 53-6.7), biotinylated anti-ICOS (BD Pharmingen, catalog number 552145, clone 7E.17G9), and then washed. Blood cells were then stained with streptavidin-APC (BD Pharmingen, catalog number 554067), and washed again. Samples were analyzed by flow cytometry on a FACSCalibur from BD Biosciences.

Luxol Fast Blue Staining of Spinal Cord Sections

Luxol Fast Blue staining was performed on formalin fixed paraffin embedded spinal cord sectioned at 4 μm. Spinal cord sections were deparaffinized and hydrated to 95% ethanol. They were then stained overnight (at least 16 hours) in Luxol Fast Blue at 60° C. Excess stain was rinsed off in 95% ethanol, and the slides were washed in dH₂O. Slides were then differentiated by quickly immersing in 0.05% lithium carbonate for 10 to 20 seconds and then through several changes of 70% ethanol until gray and white matter could be distinguished. Slides were then stained with cresyl violet for 5 minutes at 37° C., rinsed in 95% ethanol, dehydrated slowly, cleared and mounted. See Sheehan (1980) Theory and Practice of Histotechnology, 2nd ed, pp. 263-264.

Listeria Infection

Mice were inoculated intravenously with 100,000 Colony Forming Units of Listeria monocytogenes (strain #43251 from ATCC) in 100 microliters of PBS. An IP injection of the monoclonal antibodies or fusion proteins (400 μg per mouse, equivalent to 20 mg/kgs, in 100 μl PBS) was started the day prior to Listeria injection; doses of 100 μg (5 mg/kg) 3 times per week were continued for 10 days following Listeria injections. Mice were monitored twice daily for signs of disease.

Generation of Listeria

Listeria virulence was maintained by serial passage in C57B1/6 mice. Fresh isolates were obtained from infected spleens, grown in liquid brain heart infusion (BHI) or on BHI agar plates (Difco Labs, Detroit, Mich.). Bacteria were washed repeatedly, resuspended in sterile PBS, and stored at −80° C. in PBS with 20% glycerol.

### Example 3

### Treatment of Lupus with CD4 Antibody in Combination with MMF

The following sets forth a series of experiments that demonstrate that a non-depleting CD4 antibody, alone and in combination with mycophenolate mofetil, is efficacious in a preclinical model of SLE.

The NZBxW F1 mouse model of SLE was described above in Example 1. In this model, preclinical efficacy of a non-depleting CD4 antibody (YTS177, described above) was compared to that of a non-binding control antibody (described above), mycophenolate mofetil (CellCept® or MMF, a current treatment), and a combination of the CD4 antibody and MMF.

Treatment of NZB×NZW mice was initiated at 9 months of age. Mice were screened for proteinuria and randomized into groups based on their proteinuria scores. At the onset of the experiment, each treatment group included 15 mice, of which 73% exhibited proteinuria levels of >300 mg/dl. (Note that this is a more severe disease state than that at which treatment was initiated in the experiments described in Example 1 above, in which only 32% of the mice were at >300 mg/dl proteinuria.) Mice were treated continuously for two months with either control Ab, the non-depleting CD4 antibody (anti-CD4), MMF (CellCept®), or a combination of non-depleting anti-CD4 and MMF. Mice were monitored for changes in proteinuria (urinalysis was performed as described above in Example 1), disease progression, and survival. The non-depleting CD4 antibody (YTS177) was delivered 3×/week at 5 mg/kg by intraperitoneal (IP) injection. MMF was given IP at either 25 mg/kg daily or 50 mg/kg daily (alone or in combination with the CD4 antibody).

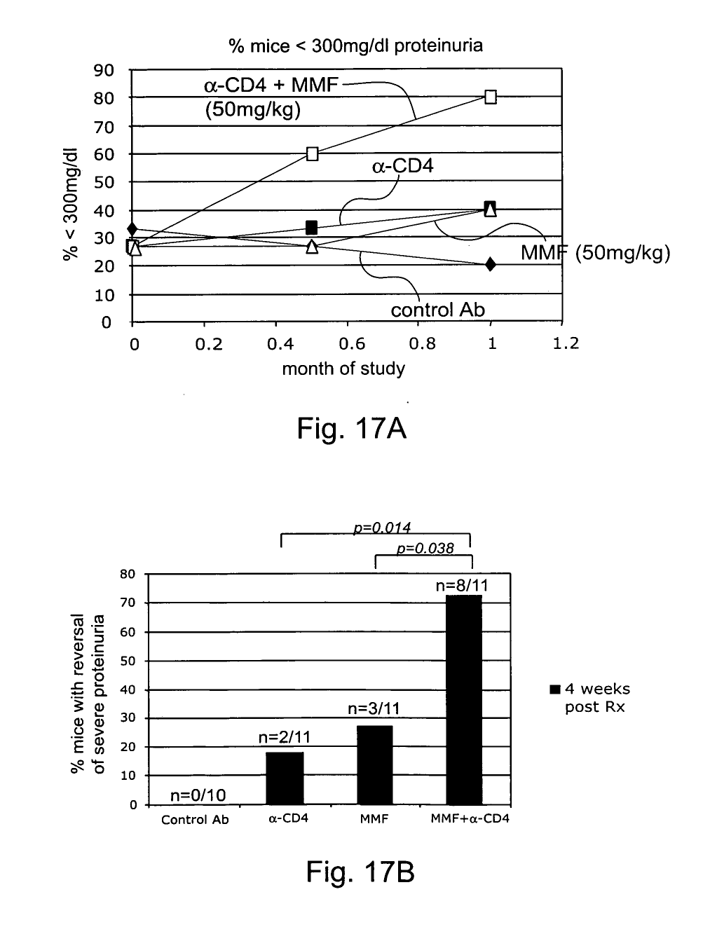

In this experiment, in which treatment was initiated at a severe disease state, the individual treatments with the CD4 antibody or with MMF were not sufficient to reverse severe proteinuria significantly (some mice improve, but the numbers are insufficient to meet significance). However, combining the treatments synergized to show a significant effect; as shown in FIG. 17, a synergistic effect in the capacity to reverse proteinuria was seen when the CD4 antibody was administered in combination with MMF. FIG. 17A illustrates the percentage of mice under 300 mg/dl proteinuria at the indicated times after treatment. Administration of the CD4 antibody in combination with MMF (CellCept®) resulted in a net decrease in mice exhibiting >300 mg/dl proteinuria, indicating a reversal of nephritis symptoms in very late stage disease that was not observed in groups treated with the control antibody or individual treatments with the CD4 antibody or MMF. FIG. 17B shows the percentage of mice reversed from 300 mg/dl proteinuria following one month of treatment.

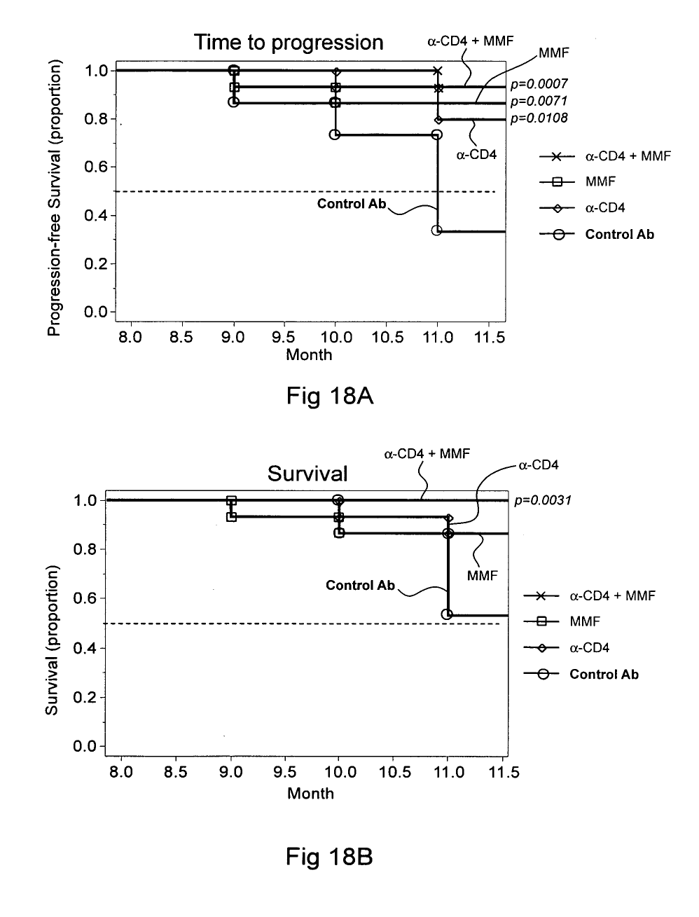

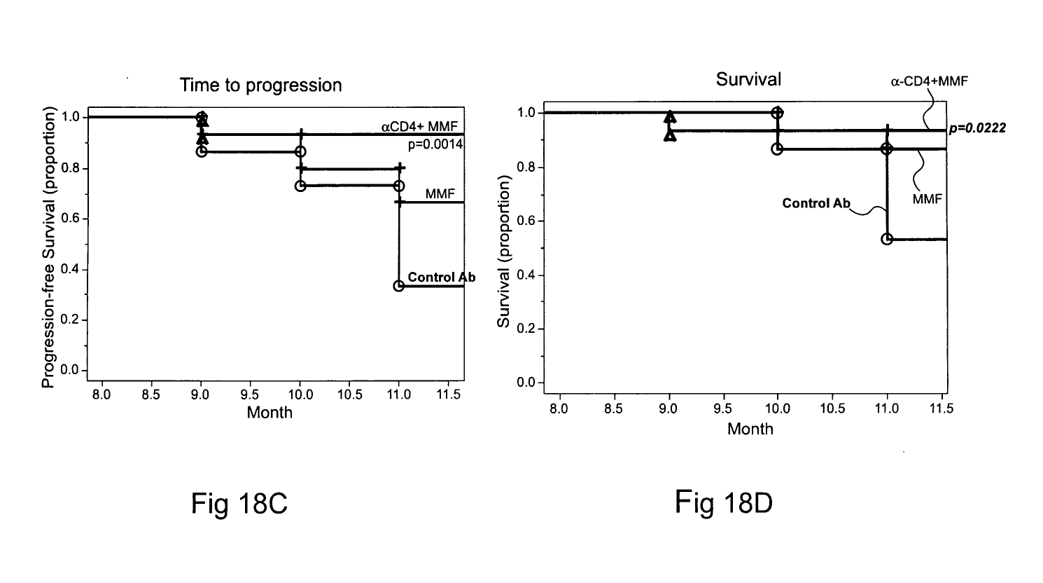

As shown in FIG. 18, administration of the non-depleting CD4 antibody in combination with MMF delayed time to progression (FIGS. 18A and 18C) and increased survival (FIGS. 18B and 18D), at both doses of MMF (50 mg/kg per day in FIGS. 18A and 18B and 25 mg/kg per day in FIGS. 18C and 18D). The combination of non-depleting CD4 antibody and MMF was more effective than either the non-depleting CD4 antibody or MMF alone. In FIGS. 18A-18D, the reference controls (control antibody-treated group) are designated in bold, and only p values for groups that achieve statistical significance vs. the reference control are designated on the graph.

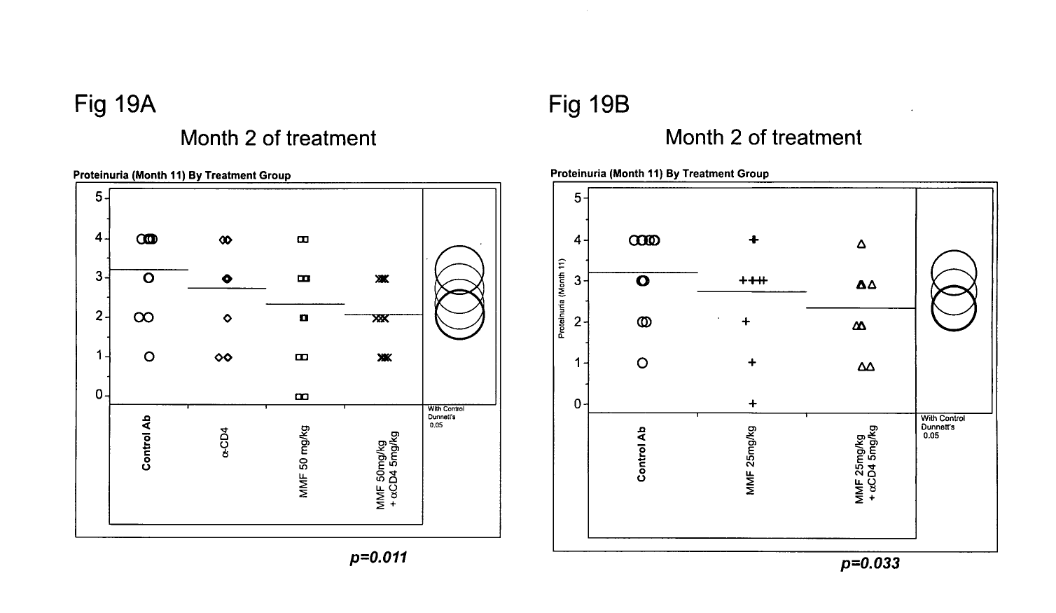

Treatment with a combination of the CD4 antibody and MMF was effective in decreasing proteinuria. FIG. 19 illustrates multiple comparison analysis of proteinuria at month 2 of treatment, using Dunnett's method with the control antibody treated group as the reference control group. Results for groups treated with 50 mg/kg daily of MMF alone or in combination with the CD4 antibody are presented in FIG. 19A, while results for groups treated with 25 mg/kg daily of MMF alone or in combination with the CD4 antibody are presented in FIG. 19B. The reference control (control antibody-treated) is designated in bold, and only p values for groups that achieve statistical significance vs. the reference control are designated on the graphs. The results demonstrate that the combination of the CD4 antibody and MMF provided significant benefit in decreasing proteinuria in the model, while treatment with control antibody, anti-CD4 alone or MMF alone did not show statistically significant reduction in proteinuria.

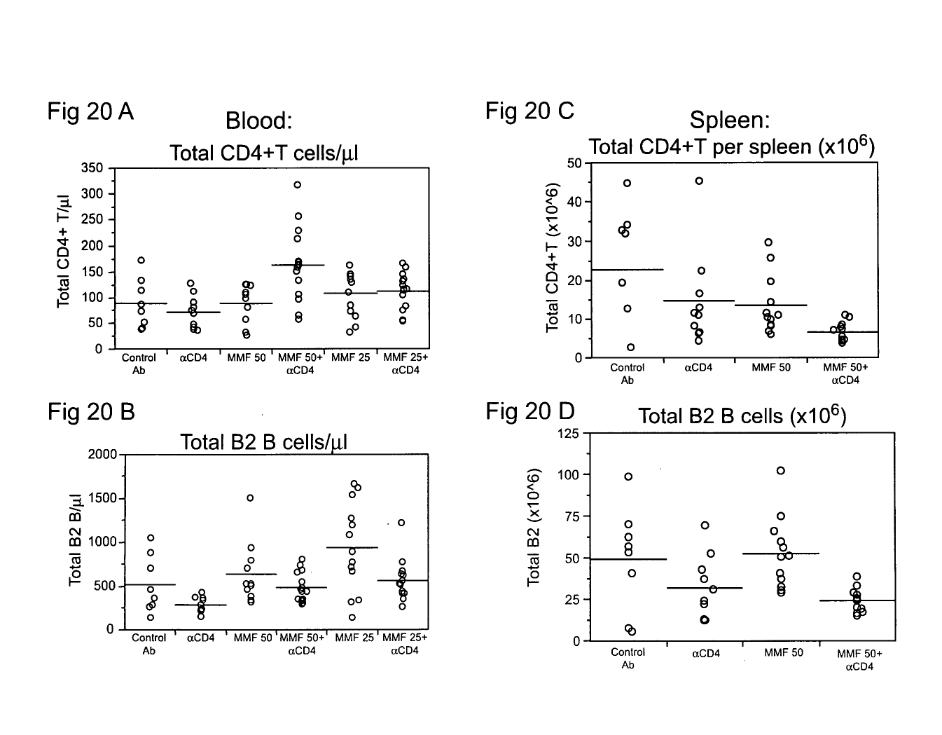

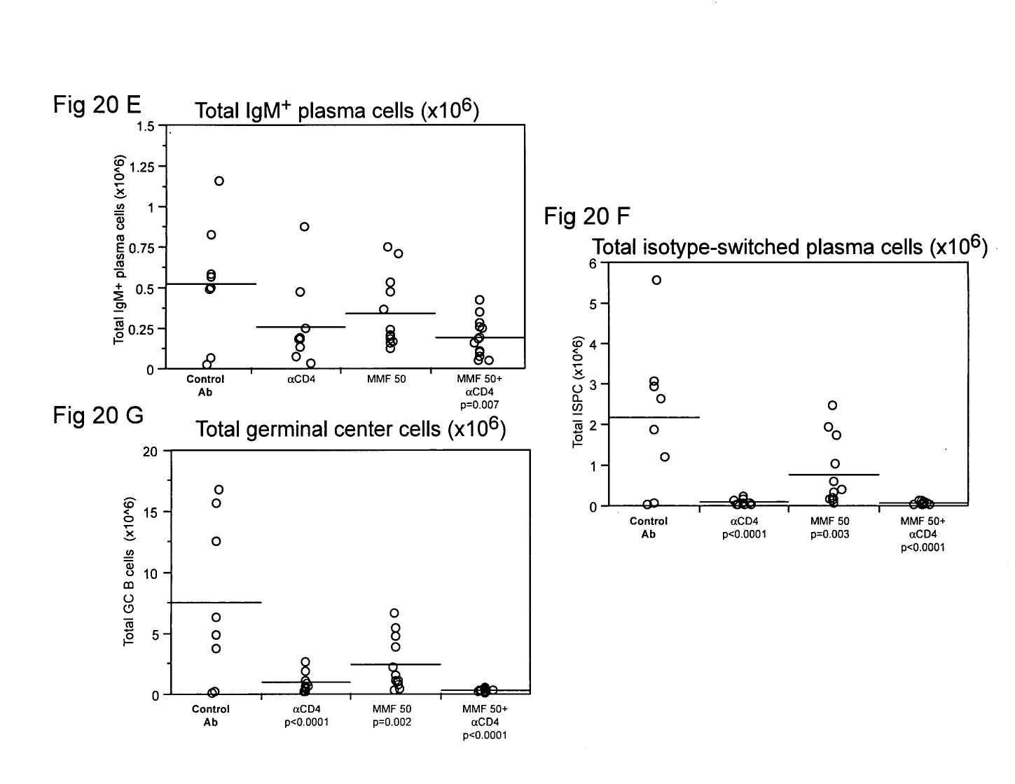

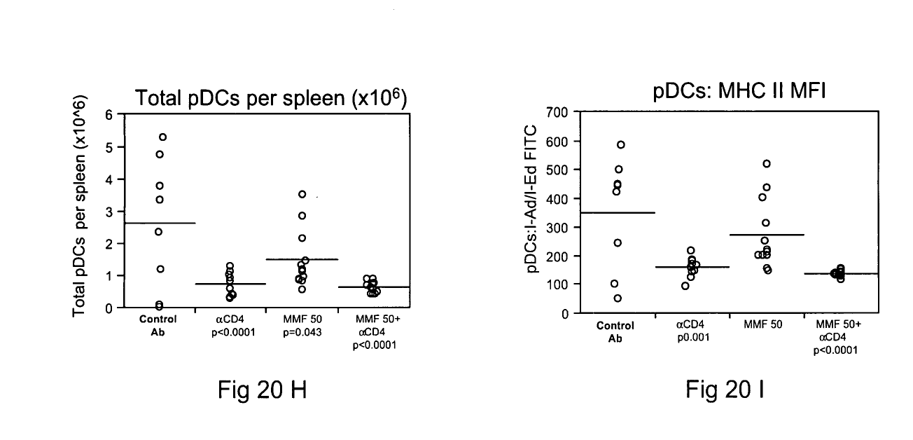

Treatment with a combination of the non-depleting CD4 antibody and MMF decreased the number of CD4⁺ T cells found in the spleen. As shown in FIG. 20C, the number of splenic CD4⁺ T cells was reduced in animals treated for two months with the combination as compared to control antibody treated animals (p=0.002). Effects of treatment with the combination were also observed downstream, for example, in B cell and dendritic cell populations. For example, treatment with a combination of the CD4 antibody and MMF decreased the number of B2 B cells found in the spleen, as shown in FIG. 20D (relative to control antibody treated animals; p=0.017). The reduction in splenic CD4⁺ T cells and B2 B cells was not due to depletion of the cells by the antibody, as evidenced by total CD4⁺ T cell and B2 B cell blood counts (FIGS. 20A and 20B, respectively). In fact, an increase in blood CD4⁺ T cell and B2 B cell numbers was noted in the groups treated with 50 mg/kg MMF in combination with the CD4 antibody and with 25 mg/kg MMF, respectively (although these increases may not be statistically significant). CD4⁺ T cell and B2 B cell numbers were determined by flow cytometry basically as described above, using antibodies to identify the various cell populations and then using the percentage of each population represented (of total lymphocytes) multiplied by the total number of lymphocytes to determine the number of each population. B2 cells (the majority of B cells) were identified by positive staining for B220 (CD45) and CD38. Anti-B220/CD45 and anti-CD38 were from BD Pharmingen.

Treatment with a combination of the non-depleting CD4 antibody and MMF also decreased the number of IgM⁺ plasma cells, as illustrated in FIG. 20E. The number of IgM⁺ plasma cells was determined by flow cytometry basically as described above. Plasma cells were identified by their expression of syndecan-1; the IgM plasma cells were those syndecan-1 positive cells that also expressed IgM on their surface. Antibodies to syndecan-1 and IgM were from BD Pharmingen. Comparisons were performed using Dunnett's method with the control antibody treated group as the reference control group (designated in bold), and only p values for groups that achieve statistical significance vs. the reference control are designated on the graph.

Similarly, treatment with the combination of the CD4 antibody and MMF decreased the number of isotype-switched plasma cells, as shown in FIG. 20F. The number of isotype-switched plasma cells was determined by flow cytometry basically as described above. Plasma cells were identified by their expression of syndecan-1; the syndecan-1 positive cells that were negative for IgM expression were the isotype-switched plasma cells (expressing isotypes other than IgM, e.g., IgG, IgE, etc.). Antibodies to syndecan-1 and IgM were from BD Pharmingen. Comparisons were performed using Dunnett's method with the control antibody treated group as the reference control group (designated in bold), and only p values for groups that achieve statistical significance vs. the reference control are designated on the graph.

Treatment with the combination also reduced the number of germinal center B cells, as shown in FIG. 20G. Germinal center B cell number was determined by flow cytometry basically as described above. Germinal center B cells were identified as those cells positive for B220 and negative for CD38 surface expression (distinguishing them from B2 cells, which co-express B220 and CD38). Anti-B220/CD45 and anti-CD38 were from BD Pharmingen. Comparisons were performed using Dunnett's method with the control antibody treated group as the reference control group (designated in bold), and only p values for groups that achieve statistical significance vs. the reference control are designated on the graph.

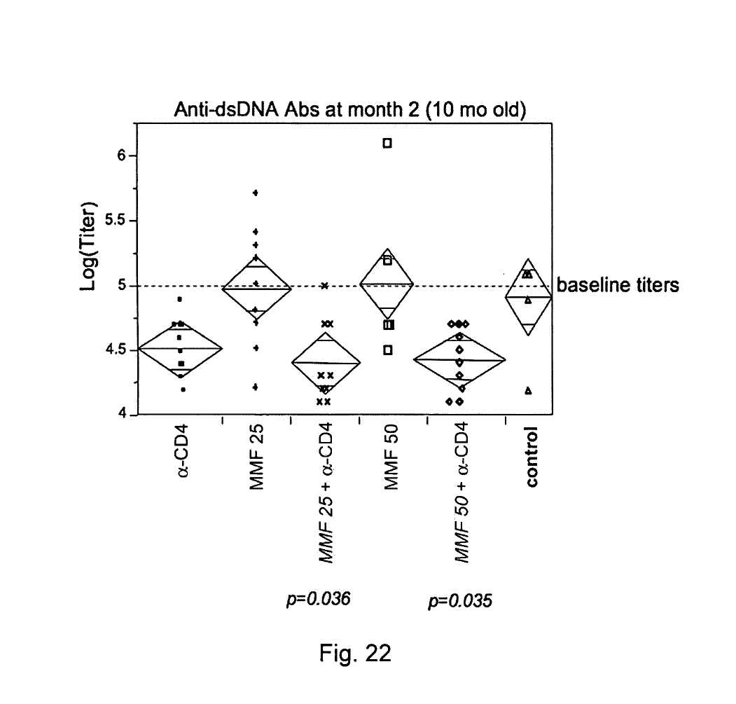

Consistent with the inhibitory effects observed on B cell populations with the anti-CD4/MMF combined treatment, a reduction in autoantibodies (anti-dsDNA titers) was seen when anti-CD4 was used in combination with either dose of MMF (FIG. 22). A trend toward reduced titers was observed with anti-CD4 treatment alone, but this did not reach statistical significance (p=0.1). Anti ds-DNA antibody titers were determined by ELISA as described above. Baseline titers prior to treatment are indicated by the dotted line in FIG. 22. Comparisons were performed using Dunnett's method with the control antibody treated group as the reference control group (designated in bold), and only p values for groups that achieve statistical significance vs. the reference control are designated on the graph. These observations demonstrate that, in addition to slowing the rate of autoantibody production, non-depleting anti-CD4 antibody can decrease existing autoantibody titers when administered in combination with MMF late in disease.

Plasmacytoid dendritic cells are potentially important drivers of lupus due to their secretion of high amounts of type I interferons (alpha and beta interferons). It is therefore worth noting that treatment with the CD4 antibody, alone or in combination with MMF, reduced the number of splenic plasmacytoid dendritic cells, as shown in FIG. 20H. Plasmacytoid dendritic cell number was determined by flow cytometry basically as described above. B and T cells were excluded using markers CD19 and CD3, respectively; of the remaining cells, plasmacytoid dendritic cells were identified based on their unique expression of pDCA and their intermediate expression of B220. Antibodies were from BD Pharmingen, except anti-pDCA which was from Miltenyi. Comparisons were performed using Dunnett's method with the control antibody treated group as the reference control group (designated in bold), and only p values for groups that achieve statistical significance vs. the reference control are designated on the graph.

Furthermore, treatment with the antibody (alone or in combination with MMF) reduced expression levels of MHC Class II in these dendritic cells, as shown in FIG. 20I. Plasmacytoid dendritic cells were identified by flow cytometry basically as described above, using an antibody directed to pDCA (Miltenyi), and their MHC II levels were assessed with an antibody directed to a common epitope in IA^{d} and IE^{d} MHC II molecules (BD Pharmingen). Comparisons were performed using Dunnett's method with the control antibody treated group as the reference control group (designated in bold), and only p values for groups that achieve statistical significance vs. the reference control are designated on the graph. Since MHC II levels are usually linked to the activation status of these dendritic cells, with increased levels indicating an increased activation state, this observation indicates that treatment with the CD4 antibody can reduce the activation status of this dendritic cell population.

In summary, treatment with the non-depleting CD4 antibody, e.g., in combination with MMF, was efficacious in NZBxW F1 mice even when introduced late in disease. Treatment with the combination extended disease-free progression and survival and decreased splenic CD4+ T cell numbers. In addition, combining the non-depleting CD4 antibody with MMF provided significant benefit over MMF alone in reversing proteinuria the NZB/W F1 model of SLE, as well as in decreasing autoantibody titers. The non-depleting CD4 antibody alone was also able to selectively reduce numbers of germinal center B cells and isotype-switched plasma cells without significantly affecting the majority of B cells (B2 cells). In addition, anti-CD4 was able to reduce the numbers of plasmacytoid dendritic cells, cells which have been linked to pathogenesis of SLE through their production of Type 1 interferons and IFN-alpha and beta.

While the foregoing invention has been described in some detail for purposes of clarity and understanding, it will be clear to one skilled in the art from a reading of this disclosure that various changes in form and detail can be made without departing from the true scope of the invention. For example, all the techniques and compositions described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of treating lupus in a mammalian subject, the method comprising:
administering to the subject a therapeutically effective amount of a combination of a non-depleting CD4 antibody and at least a second compound selected from the group consisting of: cyclophosphamide, mycophenolate mofetil, and CTLA4-Ig.

2. The method of claim 1, wherein the subject is a human.

3. The method of claim 1, wherein the second compound is cyclophosphamide.

4. The method of claim 1, wherein the antibody comprises a CDR having the amino acid sequence of SEQ ID NO:27.

5. The method of claim 1, wherein the antibody comprises a CDR having the amino acid sequence of SEQ ID NO:30.

6. The method of claim 1, wherein the antibody comprises CDRs having the amino acid sequence of SEQ ID NO:25, SEQ ID NO:26, and SEQ ID NO:27.

7. The method of claim 1, wherein the antibody comprises CDRs having the amino acid sequence of SEQ ID NO:28, SEQ ID NO:29, and SEQ ID NO:30.

8. The method of claim 1, wherein the antibody comprises CDRs having the amino acid sequence of SEQ ID NO:25, SEQ ID NO:26, SEQ ID NO:27, SEQ ID NO:28, SEQ ID NO:29, and SEQ ID NO:30.

9. The method of claim 1, wherein the antibody comprises a light chain amino acid sequence set forth in SEQ ID NO:3 and a heavy chain amino acid sequence set forth in SEQ ID NO:6, a light chain amino acid sequence set forth in SEQ ID NO:9 and a heavy chain amino acid sequence set forth in SEQ ID NO:12, a light chain amino acid sequence set forth in SEQ ID NO:15 and a heavy chain amino acid sequence set forth in SEQ ID NO:18, or a light chain amino acid sequence set forth in SEQ ID NO:21 and a heavy chain amino acid sequence set forth in SEQ ID NO:24.

10. The method of claim 9, wherein the subject is a human and wherein the second compound is cyclophosphamide.

11. The method of claim 10, wherein the lupus is lupus nephritis.

12. The method of claim 11, wherein the lupus is class II lupus nephritis, class III lupus nephritis, class IV lupus nephritis, or class V lupus nephritis.

13. The method of claim 11, wherein after initiation of treatment with the combination, the subject displays a reduction in proteinuria and/or a reduction in active urinary sediment.

14. The method of claim 1, wherein the antibody comprises a CD4 binding fragment of an antibody that comprises a light chain amino acid sequence set forth in SEQ ID NO:3 and a heavy chain amino acid sequence set forth in SEQ ID NO:6, a light chain amino acid sequence set forth in SEQ ID NO:9 and a heavy chain amino acid sequence set forth in SEQ ID NO:12, a light chain amino acid sequence set forth in SEQ ID NO:15 and a heavy chain amino acid sequence set forth in SEQ ID NO:18, or a light chain amino acid sequence set forth in SEQ ID NO:21 and a heavy chain amino acid sequence set forth in SEQ ID NO:24.

15. The method of claim 1, wherein the antibody is a CD4 antibody that binds the same epitope as an antibody selected from the group consisting of: an antibody comprising a light chain amino acid sequence set forth in SEQ ID NO:3 and a heavy chain amino acid sequence set forth in SEQ ID NO:6, an antibody comprising a light chain amino acid sequence set forth in SEQ ID NO:9 and a heavy chain amino acid sequence set forth in SEQ ID NO:12, an antibody comprising a light chain amino acid sequence set forth in SEQ ID NO:15 and a heavy chain amino acid sequence set forth in SEQ ID NO:18, and an antibody comprising a light chain amino acid sequence set forth in SEQ ID NO:21 and a heavy chain amino acid sequence set forth in SEQ ID NO:24.

16. The method of claim 1, wherein the antibody is a humanized antibody.

17. The method of claim 1, wherein the antibody has an aglycosylated Fc portion.

18. The method of claim 1, wherein the antibody does not bind to the Fc receptor.

19. The method of claim 1, wherein the antibody comprises an amino acid substitution at one or more of amino acid positions 270, 322, 326, 327, 329, 313, 333, and/or 334 of the Fc region, which substitution alters C1 q binding and/or complement-dependent cytotoxicity.

20. The method of claim 1, wherein the antibody comprises a salvage receptor binding epitope.

21. The method of claim 1, wherein the antibody comprises a serum albumin binding peptide.

22. The method of claim 1, wherein the antibody comprises three or more antigen-binding sites.

23. The method of claim 1, wherein the lupus is systemic lupus erythematosus.

24. The method of claim 1, wherein the lupus is cutaneous lupus erythematosus.

25. The method of claim 1, wherein the lupus is lupus nephritis.

26. The method of claim 25, wherein the lupus is class II lupus nephritis, class III lupus nephritis, class IV lupus nephritis, or class V lupus nephritis.

27. The method of claim 25, wherein after initiation of treatment with the combination, the subject displays a reduction in proteinuria and/or a reduction in active urinary sediment.

28. The method of claim 1, wherein, prior to initiation of treatment with the combination, the subject displays proteinuria, which proteinuria is ameliorated by the treatment.

29. The method of claim 1, wherein the second compound is mycophenolate mofetil, and wherein after initiation of treatment with the combination, the subject displays a reduction in anti-double-stranded DNA antibody titer.

30. The method of claim 1, wherein, after initiation of treatment with the combination, the lupus is ameliorated; the method comprising, after observation of the amelioration, discontinuing treatment of the subject with the combination and administering to the subject a therapeutically effective amount of the non-depleting CD4 antibody.

31. The method of claim 1, wherein, after initiation of treatment with the combination, the lupus is ameliorated; the method comprising, after observation of the amelioration, discontinuing treatment of the subject with the combination and administering to the subject a therapeutically effective amount of the second compound or one or more other compounds.

32. A method of treating lupus nephritis in a mammalian subject, the method comprising:
administering to the subject a therapeutically effective amount of a non-depleting CD4 antibody, wherein after initiation of treatment with the antibody the subject displays an improvement in renal function, a reduction in proteinuria, and/or a reduction in active urinary sediment.

33. The method of claim 32, wherein the subject is a human.

34. The method of claim 33, wherein, prior to initiation of treatment with the antibody, the subject displays proteinuria greater than 500 mg per day, greater than 1000 mg per day, greater than 2000 mg per day, or greater than 3500 mg per day, which proteinuria is reduced after initiation of treatment with the antibody.

35. The method of claim 32, wherein the antibody is selected from the group consisting of:
a) an antibody that comprises a light chain amino acid sequence set forth in SEQ ID NO:3 and a heavy chain amino acid sequence set forth in SEQ ID NO:6;
b) an antibody that comprises a light chain amino acid sequence set forth in SEQ ID NO:9 and a heavy chain amino acid sequence set forth in SEQ ID NO:12;
c) an antibody that comprises a light chain amino acid sequence set forth in SEQ ID NO:15 and a heavy chain amino acid sequence set forth in SEQ ID NO:18;
d) an antibody that comprises a light chain amino acid sequence set forth in SEQ ID NO:21 and a heavy chain amino acid sequence set forth in SEQ ID NO:24;
e) an antibody that comprises a CD4 binding fragment of the antibody of a), b), c), or d);
f) an antibody that comprises a CDR having the amino acid sequence of SEQ ID NO:27;
g) an antibody that comprises a CDR having the amino acid sequence of SEQ ID NO:30;
h) an antibody that comprises CDRs having the amino acid sequence of SEQ ID NO:25, SEQ ID NO:26, and SEQ ID NO:27;
i) an antibody that comprises CDRs having the amino acid sequence of SEQ ID NO:28, SEQ ID NO:29, and SEQ ID NO:30; and
j) an antibody that comprises CDRs having the amino acid sequence of SEQ ID NO:25, SEQ ID NO:26, SEQ ID NO:27, SEQ ID NO:28, SEQ ID NO:29, and SEQ ID NO:30.

36. The method of claim 32, wherein the antibody is a CD4 antibody that binds the same epitope as an antibody selected from the group consisting of: an antibody comprising a light chain amino acid sequence set forth in SEQ ID NO:3 and a heavy chain amino acid sequence set forth in SEQ ID NO:6, an antibody comprising a light chain amino acid sequence set forth in SEQ ID NO:9 and a heavy chain amino acid sequence set forth in SEQ ID NO:12, an antibody comprising a light chain amino acid sequence set forth in SEQ ID NO:15 and a heavy chain amino acid sequence set forth in SEQ ID NO:18, and an antibody comprising a light chain amino acid sequence set forth in SEQ ID NO:21 and a heavy chain amino acid sequence set forth in SEQ ID NO:24.

37. The method of claim 32, wherein the lupus is class II lupus nephritis, class III lupus nephritis, class IV lupus nephritis, or class V lupus nephritis.

38. A method of treating lupus in a mammalian subject, the method comprising:
administering to the subject a non-depleting CD4 antibody in an amount effective to decrease titer of one or more autoantibodies.

39. The method of claim 38, wherein the non-depleting CD4 antibody is administered in an amount effective to decrease anti-double-stranded DNA antibody titer.

40. A method of decreasing autoantibody titer in a mammalian subject, the method comprising: administering to the subject a non-depleting CD4 antibody, or a combination of a non-depleting CD4 antibody and mycophenolate mofetil, in an amount effective to decrease said autoantibody titer.

41. The method of claim 40, wherein the autoantibody titer is an anti-double-stranded DNA antibody titer.

42. A method of treating a condition in a mammalian subject, the method comprising:
administering to the subject a therapeutically effective amount of a combination of a non-depleting CD4 antibody and at least a second compound selected from the group consisting of: cyclophosphamide, mycophenolate mofetil, and CTLA4-Ig;
wherein the condition is selected from the group consisting of: rheumatoid arthritis, asthma, psoriasis, transplant rejection, graft versus host disease, multiple sclerosis, Crohn's disease, ulcerative colitis, and Sjogren's syndrome.

43. The method of claim 42, wherein the condition is selected from the group consisting of: rheumatoid arthritis, asthma, psoriasis, transplant rejection, and graft versus host disease.

44. The method of claim 42, wherein the subject is a human.

45. The method of claim 42, wherein the antibody is selected from the group consisting of:
a) an antibody that comprises a light chain amino acid sequence set forth in SEQ ID NO:3 and a heavy chain amino acid sequence set forth in SEQ ID NO:6;
b) an antibody that comprises a light chain amino acid sequence set forth in SEQ ID NO:9 and a heavy chain amino acid sequence set forth in SEQ ID NO:12;
c) an antibody that comprises a light chain amino acid sequence set forth in SEQ ID NO:15 and a heavy chain amino acid sequence set forth in SEQ ID NO:18;
d) an antibody that comprises a light chain amino acid sequence set forth in SEQ ID NO:21 and a heavy chain amino acid sequence set forth in SEQ ID NO:24;
e) an antibody that comprises a CD4 binding fragment of the antibody of a), b), c), or d);
f) an antibody that comprises a CDR having the amino acid sequence of SEQ ID NO:27;
g) an antibody that comprises a CDR having the amino acid sequence of SEQ ID NO:30;
h) an antibody that comprises CDRs having the amino acid sequence of SEQ ID NO:25, SEQ ID NO:26, and SEQ ID NO:27;
i) an antibody that comprises CDRs having the amino acid sequence of SEQ ID NO:28, SEQ ID NO:29, and SEQ ID NO:30; and
j) an antibody that comprises CDRs having the amino acid sequence of SEQ ID NO:25, SEQ ID NO:26, SEQ ID NO:27, SEQ ID NO:28, SEQ ID NO:29, and SEQ ID NO:30.
